(19) United States
(12) Patent Application Publication
(10) Pub. No.: US 2023/0149295 A1
(43) Pub. Date: May 18, 2023

(54) INFLAMMATORY BOWEL DISEASE STEM CELLS, AGENTS WHICH TARGET IBD STEM CELLS, AND USES RELATED THERETO

(71) Applicants: University of Houston System, Houston, TX (US); The Board of Regents of The University of Texas System, Austin, TX (US); Tract Pharmaceuticals, Inc., West Hartford, CT (US)

(72) Inventors: Frank McKEON, Houston, TX (US); Rahul NEUPANE, Houston, TX (US); Jingzhong XIE, Houston, TX (US); Wa XIAN, Houston, TX (US); Matthew VINCENT, West Hartford, CT (US); Shan WANG, Houston, TX (US)

(73) Assignees: University of Houston System, Houston, TX (US); The Board of Regents of The University of Texas System, Austin, TX (US); Tract Pharmaceuticals, Inc., West Hartford, CT (US)

(21) Appl. No.: 17/906,027

(22) PCT Filed: Mar. 9, 2021
(86) PCT No.: PCT/US21/21450

§ 371 Date: Sep. 9, 2022

Related U.S. Application Data
(60) Provisional application No. 62/987,022, filed on Mar. 9, 2020.

(51) Int. Cl. A61K 9/00; A61K 47/34; A61K 45/06

(52) U.S. Cl. CPC A61K 9/0024; A61K 47/34; A61K 45/06

(57) ABSTRACT

The present disclosure addresses IBD from the standpoint of inhibiting or ablating pathogenic mucosal stem cells cloned from defined regions of disease in the gastrointestinal tract. In the case of Crohn's disease, for example, isolation of those stem cells according to the methods of the present disclosure reveals a pattern of inflammatory gene expression in stem cells from the terminal ileum and colon that is epigenetically maintained despite months of continuous cultivation in the absence of immune or stromal cells, or of intestinal microbes. Superimposed on this distributed inflammatory phenotype is a differentiation defect that profoundly and specifically alters the mucosal barrier properties of the terminal ileum. The co-existence of diseased and normal stem cells within the same endoscopic biopsies of Crohn's disease patients implicates an epigenetically enforced heterogeneity among mucosal stem cells in the dynamics of this condition.

### PRIORITY CLAIM

This application claims benefit of priority to U.S. Provisional Application Ser. No. 62/987,022, filed Mar. 9, 2020, the entire contents of which are hereby incorporated by reference.

### FEDERAL FUNDING SUPPORT CLAUSE

This invention was made with government support under DK115445 awarded by National Institutes of Health. The government has certain rights in the invention.

### BACKGROUND

Inflammatory Bowel Disease (IBD) describes chronic inflammatory conditions of the digestive tract that afflict 700,000 individuals in the US. Chief among these conditions is Crohn's disease (CD), where inflammation is typically localized to the distal small intestine or ileum and colon, but can appear anywhere in the gastrointestinal tract, and ulcerative colitis (UC), in which the inflammation is restricted to the colon. A truly tragic feature of IBD is that it typically presents in individuals in their 20's and 30's, and thus its chronic nature has a major impact on patients and their families. Even worse, there is increasing recognition of cases of IBD in pediatric patients rendering these individuals at even higher risk than adult-onset cases for debilitating disease and even IBD-associated colon cancers. In the broadest sense, about 50% of IBD patients have disease of limited severity and often enter remission with little long-term consequence. For the other 50%, IBD represents a protracted battle with chronic disease that goes through cycles of flare-ups and remissions that are relatively resistant to medical therapies and will require surgery.

IBD treatments aim at controlling inflammatory symptoms, conventionally using corticosteroids, aminosalicylates and standard immunosuppressive agents such as azathioprine (6-mercaptopurine), methotrexate and ciclosporine. Of these, the only disease-modifying therapies are the immunosuppressive agents azathioprine and methotrexate, both of which have a slow onset of action and only a moderate efficacy. Long-term therapy may cause liver damage (fibrosis or cirrhosis) and bone marrow suppression. Also patients often become refractory to such treatment. Other therapeutic regimes merely address symptoms.

The complexity of the intestine—an epithelial lining admixed with immune cells poised to react against the vast microflora of the gut should they pass the epithelial barrier-almost guarantees some rate of inflammatory conditions, such as UC and CD. Indeed the monozygotic twin studies show only a 10-15% concordance rate for UC, with somewhat higher rates (30-35%) for Crohn's, numbers that pale in comparison to schizophrenia or autism. Thus other factors, including environmental factors, such as smoking, and the particular composition of the population of microorganisms in the gut, are clearly merging with the genetics. International efforts in genome-wide association studies (GWAS) are beginning to reveal the full range of genes that are linked to patients with UC and CD, with a full 30% overlap in these two diseases. The associated genes include those linked to inappropriate immune responses, such as genes associated with the activation of an unusual set of T helper cells (Th17) associated with inflammatory and autoimmune conditions such as multiple sclerosis, rheumatoid arthritis, and juvenile diabetes. However, the non-immune pathways uncovered by the GWAS studies are more surprising and certainly less vetted in IBD research. These include genes involved in intestinal cell junctional integrity essential for barrier function to gut pathogens, epithelial regeneration, and innate immune responses within the intestinal epithelia, as well as a host of cellular activities including autophagy, ER stress responses, and metabolism that could well be factors regulating intestinal cell homeostasis. Thus as much as the therapies for IBD are decidedly immune system-based, the GWAS studies are pointing to the possibility that the intestinal epithelia may be key and perhaps primary players at the foundation of both CD and UC.

These recent scientific discoveries explained why the overall rates of surgical intervention in IBD has yet to show a downward inflection despite introducing the immunosuppressants and anti-TNF-alpha therapies. This stark fact underscores our inability to reliably control either UC or CD apart from surgical options which in themselves result in multiple complications. It also raises the possibility that present-day treatments, all of which are focused on dampening the immune response, may not be directed at the fundamental basis of IBD. Consequently, there is currently no satisfactory treatment, as the cause for IBD remains unclear although infectious and immunologic mechanisms have been proposed.

### SUMMARY

One aspect of the present disclosure provides a method for treating a patient suffering from chronic inflammatory injury, metaplasia, dysplasia or cancer of gastrointestinal tissue, which method comprises administering to the patient an agent that selectively kills or inhibits the proliferation or differentiation of pathogenic epithelial stem cells (PESCs) in the gastrointestinal tissue relative to normal epithelial stem cells in GI tissue in which the PESC is found. Representative GI epithelial tissues include terminal ileum, as well as the anus and other areas where perinal disease may be manifest.

Another aspect of the disclosure provides a method of reducing proliferation, survival, migration, or colony formation ability of PESCs in a subject in need thereof comprising contacting the PESC with a therapeutically effective amount of an anti-PESC agent that selectively kills or inhibits the proliferation or differentiation of a PESC population relative to normal epithelial stem cells in the tissue in which the PESCs is found.

Another aspect of the disclosure provides a pharmaceutical preparation for treating one or more of chronic inflammatory injury, metaplasia, dysplasia or cancer of GI epithelial tissue, which preparation comprises an anti-PESC agent that selectively kills or inhibits the proliferation or differentiation of PESCs relative to normal epithelial stem cells.

Another aspect of the disclosure provides a pharmaceutical preparation for treating one or more of inflammatory bowel diseases such as Crohn's Disease and perianal Crohn's disease (PCD), which preparation comprises an anti-PESC agent that selectively kills or inhibits the proliferation or differentiation of PESCs relative to normal GI stem cells. In certain embodiments, the patient presents with Crohn's Disease. In certain embodiments, the patient presents with perianal disease.

Yet another aspect of the disclosure provides a drug eluting device, such as for treating inflammatory diseases and disorders of the gastrointestinal tract, as well as metaplasia, dysplasia and cancers of the gastrointestinal tract—including, but not limited to inflammatory bowel diseases including Crohn's disease, perianal Crohn's disease, ulcerative colitis (UC), microscopic colitis, diverticulosis-associated colitis, collagenous colitis, lymphocytic colitis and Behçet's disease, which device comprises drug release means including an anti-PESC agent that selectively kills or inhibits the proliferation or differentiation of PESCs relative to normal epithelial stem cells, which device when deployed in a patient positions the drug release means proximal to the luminal surface of the gastrointestinal tract (such as colon or terminal ileum) and releases the agent in an amount sufficient to achieve a therapeutically effective exposure of the luminal surface to the agent. Examples of drug eluting devices are drug eluting stents, drug eluting collars and drug eluting balloons.

In other embodiments, there are provided drug eluting devices that can be implanted proximal to the diseased portion of the luminal surface of the gastrointestinal tract (such as colon or terminal ileum), such as implanted extraluminally (i.e., submucosally or in or on the circular muscle or longitudinal muscle) rather than intraluminally.

In certain embodiments, the anti-PESC agent has an IC50 for selectively killing PESCs that is ⅕^{th} or less the IC₅₀ for killing normal epithelial stem cells in the tissue in which the PESCs are found, more preferably 1/10^{th}, 1/20^{th}, 1/50^{th}, 1/100^{th}, 1/250^{th}, 1/500^{th} or even 1/1000^{th}, or less the IC₅₀ for killing normal epithelial stem cells.

In certain embodiments, the anti-PESC agent has an IC50 for selectively killing PESCs that is ⅕^{th} or less the IC₅₀ for killing normal GI stem cells, more preferably 1/10^{th}, 1/20^{th}, 1/50^{th}, 1/100^{th}, 1/250^{th}, 1/500^{th} or even 1/1000^{th} or less the IC₅₀ for killing normal GI stem cells.

In certain embodiments, the anti-PESC agent has an IC50 for selectively killing Crohn's PESCs that is ⅕^{th} or less the IC₅₀ for killing normal terminal ileum stem cells, more preferably 1/10^{th}, 1/20^{th}, 1/50^{th}, 1/100^{th}, 1/250^{th}, 1/500^{th} or even 1/1000^{th} or less the IC₅₀ for killing normal terminal ileum stem cells.

In certain embodiments, the anti-PESC agent has an IC₅₀ for selectively inhibiting the proliferation of PESCs that is ⅕^{th} or less the IC₅₀ for inhibiting normal epithelial stem cells in the GI tissue in which the PESCs are found, more preferably 1/10^{th}, 1/20^{th}, 1/50^{th}, 1/100^{th}, 1/250^{th}, 1/500^{th} or even 1/1000^{th} or less the IC₅₀ for inhibiting the proliferation of normal epithelial stem cells.

In certain embodiments, the anti-PESC agent has an IC₅₀ for selectively inhibiting the proliferation of PESCs that is ⅕^{th} or less the IC₅₀ for inhibiting the proliferation of normal GI stem cells, more preferably 1/10^{th}, 1/20^{th}, 1/50^{th}, 1/100^{th}, 1/250^{th}, 1/500^{th} or even 1/1000^{th} or less the IC₅₀ for inhibiting the proliferation of normal GI stem cells.

In certain embodiments, the anti-PESC agent has an IC₅₀ for selectively inhibiting the proliferation of Crohn's PESCs that is ⅕^{th} or less the IC₅₀ for inhibiting the proliferation of normal terminal ileum stem cells, more preferably 1/10^{th}, 1/20^{th}, 1/50^{th}, 1/100^{th}, ½₅₀ₜₕ, 1/500^{th} or even 1/1000^{th} or less the IC₅₀ for inhibiting the proliferation of normal terminal ileum stem cells.

In certain embodiments, the anti-PESC agent has an IC₅₀ for selectively inhibiting the differentiation of PESCs that is ⅕^{th} or less the IC₅₀ for inhibiting the differentiation of normal GI stem cells, more preferably 1/10^{th}, 1/20^{th}, 1/50^{th}, 1/100^{th}, 1/250^{th}, 1/500^{th} or even 1/1000^{th} or less the IC₅₀ for inhibiting the differentiation of normal GI stem cells.

In certain embodiments, the anti-PESC agent has an IC₅₀ for selectively inhibiting the differentiation of Crohn's PESCs that is ⅕^{th} or less the IC₅₀ for inhibiting the differentiation of normal terminal ileum stem cells, more preferably 1/10^{th}, 1/20^{th}, 1/50^{th}, 1/100^{th}, 1/250^{th}, 1/500^{th} or even 1/1000^{th} or less the IC₅₀ for inhibiting the differentiation of normal terminal ileum stem cells.

In certain embodiments, the anti-PESC agent has a therapeutic index (TI) for treating inflammatory diseases and disorders of the gastrointestinal tract, or other metaplasia, dysplasia and cancers of the the gastrointestinal tract, of at least 2, and more preferably has a therapeutic index of at least 5, 10, 20, 50, 100, 250, 500 or 1000.

In certain embodiments, the anti-PESC agent has a therapeutic index (TI) for treating Crohn's Disease of at least 2, and more preferably has a therapeutic index of at least 5, 10, 20, 50, 100, 250, 500 or 1000.

In certain embodiments, the anti-PESC agent has a therapeutic index (TI) for treating Perianal Crohn's Disease of at least 2, and more preferably has a therapeutic index of at least 5, 10, 20, 50, 100, 250, 500 or 1000.

In certain embodiments, the anti-PESC agent has a therapeutic index (TI) for treating onr or more Inflammatory Bowel Diseases of at least 2, and more preferably has a therapeutic index of at least 5, 10, 20, 50, 100, 250, 500 or 1000.

In certain embodiments, the anti-PESC agent inhibits the proliferation or differentiation of GI PESCs, or kills GI PESCs, with an IC₅₀ of 10⁻⁶ M or less, more preferably 10⁻⁷ M or less, 10⁻⁸ M or less or 10⁻⁹ M or less.

In certain embodiments, the anti-PESC agent inhibits the proliferation or differentiation of Crohn's PESCs, or kills Crohn's PESCs, with an IC₅₀ of 10⁻⁶ M or less, more preferably 10⁻⁷ M or less, 10⁻⁸ M or less or 10⁻⁹ M or less.

In certain embodiments, the anti-PESC agent inhibits the proliferation or differentiation of GI PESCs, or kills GI PESCs, with an IC₅₀ of 10⁻⁶ M or less, more preferably 10⁻⁷ M or less, 10⁻⁸ M or less or 10⁻⁹ M or less.

In certain embodiments, the anti-PESC agent is administered by topical application, such as to gastrointestinal or anal tissue.

In certain embodiments, the anti-PESC agent is administered by submucosal injection, such as to gastrointestinal or anal tissue.

In certain embodiments, the anti-PESC agent is formulated for topical application, such as to gastrointestinal or anal tissue.

In certain embodiments, the anti-PESC agent is formulated as part of a bioadhesive formulation.

In certain embodiments, the anti-PESC agent is formulated as part of a drug-eluting particle, drug eluting matrix or drug-eluting gel.

In certain embodiments, the anti-PESC agent is formulated as part of a bioerodible drug-eluting particle, bioerodible drug eluting matrix or bioerodible drug-eluting gel.

In certain embodiments, the anti-PESC agent is co-administered with an analgesic, and an anti-infective or both. These may be administered as separate formulation, or optionally, may be the anti-PESC agent is co-formulated with the analgesic or the anti-infective or both.

In certain embodiments, the anti-PESC agent is formulated as a liquid for oral delivery to the gastrointestinal tissue.

In certain embodiments, the anti-PESC agent is formulated as a single oral dose.

In certain embodiments, the anti-PESC agent is delivered by a drug eluting device that is a drug eluting stent.

In certain embodiments, the anti-PESC agent is delivered by a drug eluting device that is a balloon catheter having a surface coating including the agent.

In certain embodiments, the anti-PESC agent is cell permeable, such as characterized by a permeability coefficient of 10⁻⁹ or greater, more preferably 10⁻⁸ or greater or 10⁻⁷ or greater.

In certain embodiments, the anti-PESC agent is an HSP90 inhibitor, a HSP70 inhibitor or a dual HSP90/HSP70 inhibitor.

In certain embodiments, the anti-PESC agent is an mTOR inhibitor.

In certain embodiments, the anti-PESC agent is a RAR antagonist.

In certain embodiments, the anti-PESC agent is a proteasome inhibitor, preferably an immunoproteasome inhibitor.

In certain embodiments, the anti-PESC agent is a BCR-ABL kinase inhibitor.

In certain embodiments of the methods, preparations and devices of the present disclosure the anti-PESC agent is administered with a second drug agent that selectively promotes proliferation or other regenerative and wound healing activities of normal gastrointestinal stem cells (an “ESO Regenerative agent”) with an EC₅₀ at least 5 times more potent than for PESCs, more preferably with an EC₅₀ 10 times, 50 times, 100 times or even 1000 times more potent for normal gastrointestinal stem cells (especially of the terminal ileum) relative to for PESCs. Exemplary ESO Regenerative agents include BACE inhibitors (preferably BACE1 inhibitors), FAK Inhibitors, VEGFR inhibitor or AKT inhibitor.

In certain embodiments of the methods, preparations and devices of the present disclosure the anti-PESC agent is administered with a second drug agent that selectively promotes proliferation or other regenerative and wound healing activities of normal epithelial stem cells (an “ESO Regenerative agent”) with an EC₅₀ at least 5 times more potent than for PESCs, more preferably with an EC₅₀ 10 times, 50 times, 100 times or even 1000 times more potent for normal epithelial stem cells relative to for PESCs.

In certain embodiments of the methods, preparations and devices of the present disclosure the anti-PESC agent is administered with an ESO Regenerative agent selectively promotes proliferation of normal GI stem cells with an EC₅₀ of 10⁻⁶ M or less, more preferably 10⁻⁷ M or less, 10⁻⁸ M or less or 10⁻⁹ M or less.

In certain embodiments of the methods, preparations and devices of the present disclosure the anti-PESC agent is administered with an ESO Regenerative agent selectively promotes proliferation of normal epithelial stem cells with an EC₅₀ of 10⁻⁶ M or less, more preferably 10⁻⁷ M or less, 10⁻⁸ M or less or 10⁻⁹ M or less.

In certain embodiments, the combined administration of the anti-PESC agent and the ESO Regenerative agent has a therapeutic index (TI) for treating an Inflammatory Bowel Disease (such as Crohn's Disease or Perinal Crohn's Disease) and/or gastrointestinal cancer of at least 2, and more preferably has a therapeutic index of at least 5, 10, 20, 50, 100, 250, 500 or 1000.

In certain embodiments, the second drug agent the anti-PESC agent and the ESO Regenerative agent are administered to the patient as separate formulations.

In certain embodiments, the second drug agent the anti-PESC agent and the ESO Regenerative agent are co-formulated together.

In certain embodiments, the disclosure provides a terminal ileum retentive formulation comprising (i) an agent that selectively kills or inhibits the proliferation or differentiation of pathogenic epithelial stem cells (PESCs) relative to normal gastrointestinal stem cells, (ii) a bioadhesive, and (iii) optionally, one or more pharmaceutically acceptable excipients.

For instance, the formulation can have a mucosal residence half-life on terminal ileum tissue of at least 120 minutes.

For instance, the formulation can produce at least a minimally effective concentration (MEC) of the agent in terminal ileum tissue to which it is applied for at least 120 minutes.

For instance, the formulation can produce agent concentration in terminal ileum tissue to which it is applied with T_{1/2} of at least 4 hours.

In another embodiments, there is a provided a perianal or anorectal retentive formulation comprising (i) an agent that selectively kills or inhibits the proliferation or differentiation of pathogenic epithelial stem cells (PESCs) relative to normal gastrointestinal stem cells, (ii) a bioadhesive, and (iii) optionally, one or more pharmaceutically acceptable excipients Preferably, there is provided a perinal retentive formulation.

For instance, the perianal or anorectal retentive formulation can have a perinal or anorectal tissue of at least 120 minutes.

For instance, the perianal or anorectal retentive formulation can produce at least a minimally effective concentration (MEC) of the agent in perianal or anorectal tissue to which it is applied for at least 120 minutes.

For instance, the perianal or anorectal retentive formulation can produce agent concentration in perianal or anorectal tissue to which it is applied with T_{1/2} of at least 4 hours.

For instance, the perianal or anorectal retentive formulation is a viscous bioadhesive liquid to coat the perianal or anorectal tissue.

For instance, the perianal or anorectal retentive formulation can comprise agent eluting multiparticulates, microparticles, nanoparticles or microdiscs.

For instance, the perianal or anorectal retentive formulation can include one or more an HSP90 inhibitor, an HSP70 inhibitor, a dual HSP90/HSP70 inhibitor, an mTOR inhibitor, an RAR antagonist, a proteasome inhibitor, an EGFR inhibitor, a ROCK inhibitor, a MELK inhibitor, a SRC inhibitor and/or a BCR-ABL kinase inhibitor.

For instance, the perianal or anorectal retentive formulation can further include one or more a BACE inhibitor, an FAK inhibitor, a VEGR inhibitor and/or an AKT inhibitor.

In further embodiments, there is provided bioadhesive nanoparticle having a polymeric surface with an adhesive force equivalent to an adhesive force of between 10 N/m² and 100,000 N/m² measured on human mucosal surfaces, which nanoparticle further includes: (i) a first agent selected from an HSP90 inhibitor, an HSP70 inhibitor, a dual HSP90/HSP70 inhibitor, an mTOR inhibitor, an RAR antagonist, a proteasome inhibitor, an EGFR inhibitor, a ROCK inhibitor, a MELK inhibitor, a SRC inhibitor or a BCR-ABL kinase inhibitor; and (ii) a second agent selected from a BACE inhibitor, an FAK inhibitor, a VEGR inhibitor or an AKT inhibitor, the first and second agents dispersed therein or thereon, wherein the nanoparticle elutes the first and second agents into the mucous gel layer when adhered to mucosal tissue.

In still other embodiments, there is provided a submucosal retentive formulation comprising: (i) a first agent selected from an HSP90 inhibitor, an HSP70 inhibitor, a dual HSP90/HSP70 inhibitor, an mTOR inhibitor, an RAR antagonist, a proteasome inhibitor, an EGFR inhibitor, a ROCK inhibitor, a MELK inhibitor, a SRC inhibitor or a BCR-ABL kinase inhibitor; (ii) a second agent selected from a BACE inhibitor, an FAK inhibitor, a VEGR inhibitor or an AKT inhibitor; and (iii) one or more pharmaceutically acceptable excipients, which formulation is injectable submucosally and forms a submucusal depot releasing an effective amount of the first and second agents to the surrounding tissue.

In other embodiments, there is provided an injectable thermogel for submucosal injection, comprising: (i) a first agent selected from an HSP90 inhibitor, an HSP70 inhibitor, a dual HSP90/HSP70 inhibitor, an mTOR inhibitor, an RAR antagonist, a proteasome inhibitor, an EGFR inhibitor, a ROCK inhibitor, a MELK inhibitor, a SRC inhibitor or a BCR-ABL kinase inhibitor; (ii) a second agent selected from a BACE inhibitor, an FAK inhibitor, a VEGR inhibitor or an AKT inhibitor; and optionally (iii) one or more pharmaceutically acceptable excipients, wherein the thermogel has a low-viscosity fluid at room temperature (and easily injected), and becomes a non-flowing gel at body temperature after injection.

In further embodiments, there is provided a drug eluting device for treating an inflammatory bowel disease, which device comprises drug release means including an agent that selectively kills or inhibits the proliferation or differentiation of pathogenic epithelial stem cells (PESCs) relative to normal gastrointestinal stem cells, which device when deployed in a patient positions the drug release means proximal to the luminal surface of the gastrointestinal tissue and releases the agent in an amount sufficient to achieve a therapeutically effective exposure of the luminal surface to the agent. The agent can be selected from an HSP90 inhibitor, an HSP70 inhibitor, a dual HSP90/HSP70 inhibitor, an mTOR inhibitor, an RAR antagonist, a proteasome inhibitor, an EGFR inhibitor, a ROCK inhibitor, a MELK inhibitor, a SRC inhibitor or a BCR-ABL kinase inhibitor, or a combination thereof. The drug eluting device may include a second agent selected from a BACE inhibitor, an FAK inhibitor, a VEGR inhibitor or an AKT inhibitor.

Exemplary drug eluting devices include biodegradable stents, self-expandable stents, such as a self-expandable metallic stent (SEMS) or self-expandable plastic stent (SEPS), chips and wafers for submucusal implantation, and the like.

In other embodiments, the drug eluting device is a device for extraluminal placement, such as a microneedle cuff.

The present disclosure also provides single oral dosage formulation comprising: (i) a first agent selected from an HSP90 inhibitor, an HSP70 inhibitor, a dual HSP90/HSP70 inhibitor, an mTOR inhibitor, an RAR antagonist, a proteasome inhibitor, an EGFR inhibitor, a ROCK inhibitor, a MELK inhibitor, a SRC inhibitor or a BCR-ABL kinase inhibitor; (ii) a second agent selected from a BACE inhibitor, an FAK inhibitor, a VEGR inhibitor or an AKT inhibitor; and (iii) and a pharmaceutically acceptable excipient, which single oral dosage formulation taken by an adult patient produces a concentration of the first and second agent in terminal ileum tissue effective to slow or reverse the progress of Crohn's disease.

In certain embodiments, the methods, preparations and devices of the present disclosure are intended (and appropriate) for use in human patients.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-E. Stem Cell Clonogenicity in Ground State and Organoid Culture. (FIG. 1A) Schematic of cloning human ISCs from endoscopic biopsies. Libraries of ISC colonies are generated on irradiated 3T3-J2 cells and single colonies sampled and single cell sorted to 384-well plates. Single cell-derived ISC clones are expanded and differentiated to in ALI culture to yield a 3-D epithelium marked by Muc2+ goblet cells, CHGA+ endocrine cells, and Defa6+ Paneth cells. (FIG. 1B) Maintenance of high rates of clonogenicity of ground state ISCs across serial passages as monitored by single cell-derived colony formation in 384-well plates. (FIG. 1C) Plot of cell number versus passage number at theoretical 70% clonogenicity (blue) compared to empirically determined cell numbers for ground state ISCs (red). (FIG. 1D) Top, Phase-contrast micrographs of ground state colonies and organoids derived from common population of cloned, ground state ISCs. Bottom, Rhodamine red-stained colonies in ground state culture resulting from identical numbers of ground state ISCs or organoid cells. (FIG. 1E) Histogram of clonogenicity of ground state ISCs and organoid cells. Error bars, SDM.

FIGS. 2A-D. Comparative gene expression of ground state ISCs and organoids. (FIG. 2A) Expression heatmap highlighting 1150 differentially expressed genes between ground state ISC and organoids derived from the same cloned ISCs (Log 2 2-fold; p<0.05). (FIG. 2B) Histogram detailing some of the differentially expressed genes between ground state ISCs and organoid cells. (FIG. 2C) Volcano plot of differentially expressed genes between ground state ISCs and organoid cells (p<0.05) from which a pathway analysis revealed differences WNT, NOTCH, and BMP signaling (p<0.05). (FIG. 2D) Histogram of Gene Ontology (GO) biological process enriched terms of differentially expressed genes between ground state ISCs and organoid cells (p<0.05).

FIGS. 3A-C. Comparison between ISC fate in ALI differentiation and organoid culture. (FIG. 3A) Left, Expression heatmap of ground state ISCs and after adaptation to ALI and organoid culture. Right, heatmap of selected differentiation and intestine stem cell markers. (FIG. 3B) Venn diagram of genes intestinal stem cells overrepresented in ALI culture to ISCs and those overrepresented in organoid culture relative to ground state ISCs. Top, Histograms of Tissue Specificity gene sets overrepresented in ALI and organoid culture relative to ground state ISCs (p<0.05). Bottom, Histograms of Functional gene sets overrepresented in ALI and organoid culture relative to ground state ISCs (p<0.05). (FIG. 3C) Expression heatmaps of most differentially expressed genes in ALI differentiated ISCs and ISCs grown as organoids (2-fold, p<0.05).

FIGS. 4A-F. Clonal analysis of stem cell heterogeneity in CD. (FIG. 4A) Work-flow of generating “libraries” of stem cell colonies and clonal stem cell lines from 1 mm endoscopic biopsies of terminal ileum. Scale bar, 100 um. (FIG. 4B) Left, Single and merged tSNE profiles of single cell RNAseq data of control (SPN-19) and CD (SPN-29) stem cell libraries to reveal three primary clusters. Right, Mapping of cluster-specific markers onto an integrated tSNE profile assembled from control (SPN-19) and CD (SPN-29) data. (FIG. 4C) Expression heatmap of 16 marker genes assessed by RT-PCR across 275 clones sampled from 2 control and 19 Crohn's stem cell libraries. (FIG. 4D) tSNE analysis of 275 clones based on the expression data of 16 markers. Inset highlights the distribution of clones sampled from the libraries of a single Crohn's patient (SPN-29) relative to the overall clusters identified. (FIG. 4E) Phase contrast and immunofluorescence imaging of colonies from cloned CLST1, 2, and 3 lines from a single CD library using antibodies to SOX9, CEACAM5, VSIG1, and PSCA. Scale bar, 100 um. (FIG. 4F) Histogram of FACS data from 11 control libraries and 38 Crohn's libraries depicting percentages of CLST1, CLST2, and CLST3 cells.

FIGS. 5A-E. CLST2 and CLST3 clones are committed to upper GI fates. (FIG. 5A) Left, Schematic for in vitro differentiation of single cell-derived clones. Right, Epithelia derived from differentiation of CLST1, CLST2, or CLST3 clones stained with antibodies to MUC2, MUC5AC, VSIG1, and PSCA. Scale bar, 100 um. (FIG. 5B) Principal component analysis of whole genome expression data (1.5×, p<0.05) of differentiated CLST1, CLST2, and CLST3 clones from indicated patient libraries. (FIG. 5C) Expression heatmap of selected marker genes of ALI-differentiated clones, including those of goblet cells (e.g. ZG16), Paneth cells (DEFA6), endocrine cells (SST, GCG), and junctional markers (VSIG1). (FIG. 5D) Top, Histogram depicting the top 5 enriched tissues (p<0.01) determined by ARCHS4 Tissues of genes expressed in ALI-differentiated CLST1 clones. Bottom, Corresponding histogram for ALI-differentiated CLST2 and 3 clones. (FIG. 5E) Left, Schematic of transplantation of clones into immunodeficient mice, the generation of a xenograft nodule, and xenograft histology showing the staining pattern of human-specific STEM-121 antibody. Right, Histology sections of xenograft nodules of CLST1, CLST2, or CLST3 clones stained with H&E or by immunofluorescence. Scale bar, 100 um.

FIGS. 6A-F. Proinflammatory Signatures of CLST2 and CLST3. (FIG. 6A) Histology of xenografts of control and CD stem cell libraries stained with H&E or immunofluorescence of antibodies to ECAD and counterstained with DAPI for nuclei. Inset focuses on single epithelial cyst stained with H&E, and antibodies to the hematopoietic marker CD45 and the neutrophil marker LY6G. Scale bars, 100 um. (FIG. 6B) Histogram of lumen inflammation scored as low, moderate, and high across 11 control and 38 CD libraries. (FIG. 6C) Histology of xenografts resulting from transplants of individual CLST1, CLST2, or CLST3 clones showing that only CLST3 xenografts trigger infiltration by neutrophils. Scale bar, 100 um. (FIG. 6D) Expression heatmap of partial list of inflammatory genes differentially represented among CLST1, CLST2, and CLST3. (FIG. 6E) Network analysis of inflammatory genes differentially expressed in CLST2 and CLST3 stem cells versus CLST1 stem cells. (FIG. 6F) Overlap between differentially expressed genes in CLST2 and CLST3 (vs CLST1) clones and the 1,290 genes within linkage disequilibrium blocks implicated by three separate GWAS studies. Of the 206 CLST2 and CLST3 genes overlapping with the LD blocks, 75 overlap with those in the LD block implicated by the GRAIL algorithm including 53 differentially over-expressed and 22 genes differentially under-expressed.

FIGS. 7A-G. Pro-fibrotic activities of CLST2 and CLST3 clones. (FIG. 7A) Immunofluorescence detection of antibodies to a-SMA (green) and ECAD (red) on histological sections of xenografts of control and Crohn's stem cell libraries. Scale bar, 100 um. (FIG. 7B) Quantitative assessment of submucosal myofibroblasts in xenografts of 11 control and 38 Crohn's stem cell libraries. (FIG. 7C) Box plot of extent of submucosal myofibroblasts in xenografts of 11 controls and 38 Crohn's stem cell libraries. Medians, Q1, Q3, and p-values are indicated. (FIG. 7D) Histological sections of Crohn's library xenograft stained with the antihuman Stem121 antibody and antibodies to fibronectin (FN1), a marker of myofibroblasts. Scale bar, 100 um. (FIG. 7E) Immunofluorescence labeling of ECAD and a-SMA on histological sections of xenograft nodules resulting from transplants of CLST1, CLST2, and CLST3. Scale bar, 100 um. (FIG. 7F) Expression heatmap of fibrosis-related genes that are differentially expressed in whole genome expression dataset of CLST1, CLST2, and CLST3 clones. (FIG. 7G) Network analysis using differentially expressed fibrosis-related genes in FIG. 7F.

FIGS. 8A-F. CLST 2 and CLST3 variant stem cells in terminal ileum. (FIG. 8A) Brightfield image of 21-week ileum from fetus stained to H&E. (FIG. 8B) tSNE profile of scRNAseq data of stem cell library of 21-week fetus ileum and corresponding pie chart of the distribution of clone types. (FIG. 8C) Principal component analysis of whole genome expression data of nominal clone types corresponding to CLST1, CLST2, and CLST3 from both 21-week fetal ileum and from a pediatric Crohn's case (SPN-29). (FIG. 8D) Xenograft nodules formed by cloned fetal variants assessed by H&E staining and immunofluorescence using antibodies to ECAD (red) and a-SMA (green). Sections of CLST3 nodules are further stained by immunohistochemistry with antibodies to murine CD45 (mCD45) and LY6G as indicated. Scale bar, 100 um. (FIG. 8E) Co-xenografts with compensating ratios of CLST3 and CLST1 clones assessed by immunofluorescence with antibodies to ECAD (red) and a-SMA (green) and by immunohistochemistry with antibodies to mCD45. Scale bars, 100 um. (FIG. 8F) Graphical representation of percentage of CLST3 clones in stem cell libraries generated from terminal ileum biopsies of control and CD cases. Arrow indicates median percentage of CLST3 clones in CD cases.

FIGS. 9A-C. Crohn's variant stem cells similar to upper gastrointestinal tract. (FIG. 9A) Immunofluorescence of histological sections of terminal ileum biopsy of Crohn's case with antibodies to CLST1 markers MUC2 and GPA33, CLST2 and CLST3 markers VSIG1, and the CLST2 marker LCN2. Scale bar, 100 um. (FIG. 9B) Middle, Heatmap of inflammatory gene expression in stem cells derived from each segment of the human gastrointestinal tract. Left, Network analysis of inflammatory genes differentially expressed in stem cells of the gastric body (BD), antrum (AN), and duodenum (DU) compared to those of ileocolon regions of the gastrointestinal tract. Right, Network analysis of inflammatory genes differentially expressed by stem cells of the ileum and ascending colon versus more proximal and distal regions of the gastrointestinal tract. (FIG. 9C) Expression heatmaps of differentially expressed inflammatory genes common between CD variant stem cells and those of the human gastrointestinal tract.

FIGS. 10A-B. CLST2 and CLST3 clones are committed to upper GI fates. (FIG. 10A) Additional immunofluorescence labeling of ALI-differentiated CLST1, CLST2, and CLST3 clones using antibodies to CHGA (endocrine cells), Ki67 (proliferation), DEFA6 (Paneth cells), GPA33 and CEACAM5 (colonic epithelium), CLDN18 (gastric junctional marker), and LCN2. Scale bar, 100 um. (FIG. 10B) Analysis of xenografts of the same clones as in a by immunofluorescence with antibodies to ECAD, GPA33, CLDN18, and LCN2. Scale bar, 100 um.

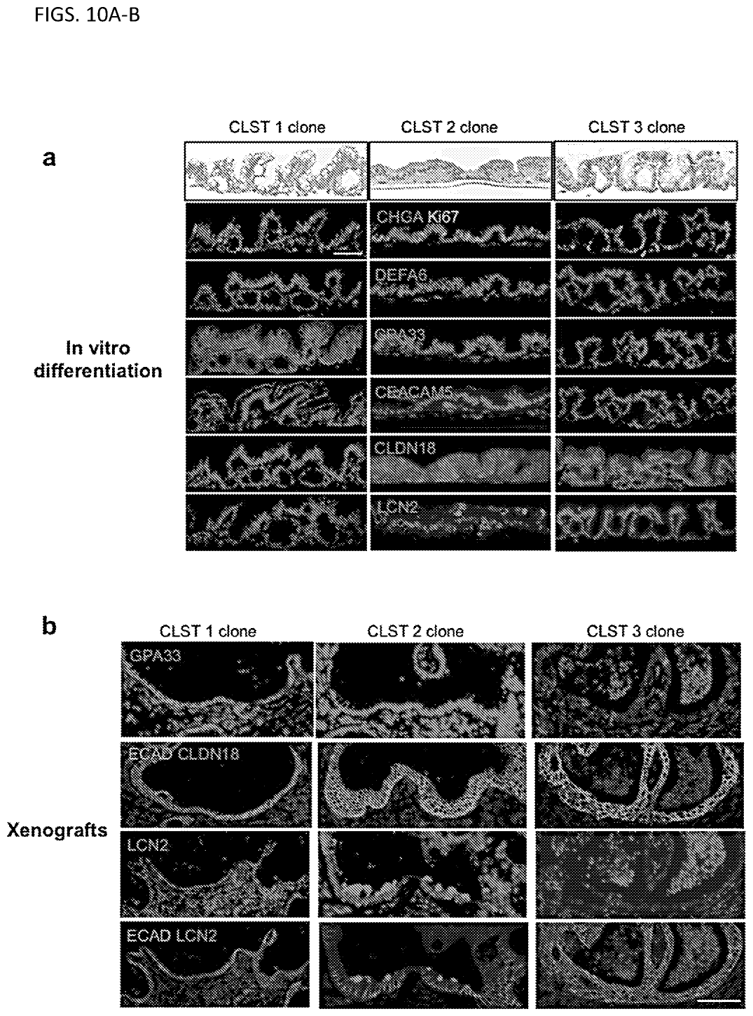

FIG. 11. Patient-matched Clones of Cluster 1 Vs Cluster 3 For Drug Discovery. Hits are shown as circles indicating the lethality to each of Cluster 1 (normal) stem cells along the x-axis and Cluster 3 (Crohn's) stem cells along the y-axis.

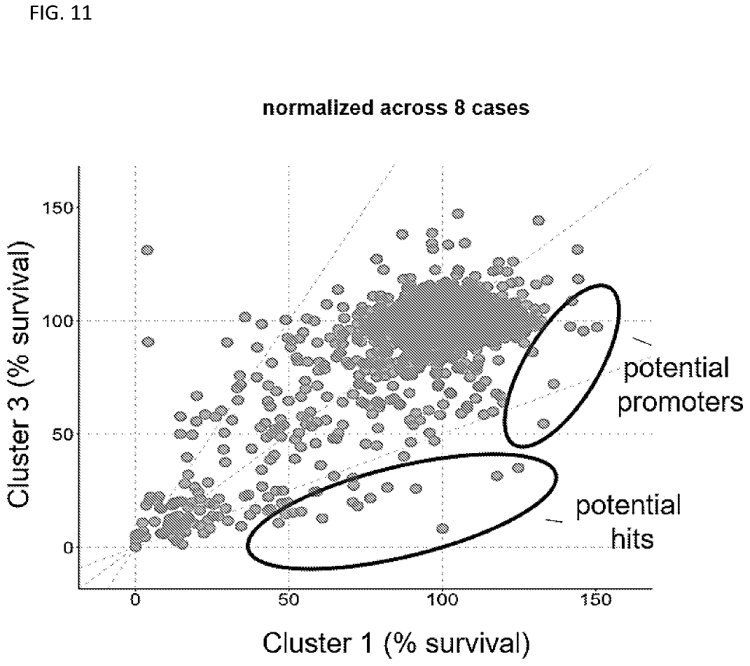

FIG. 12. Dose response curve of Single Agent Against Patient-matched Clones.

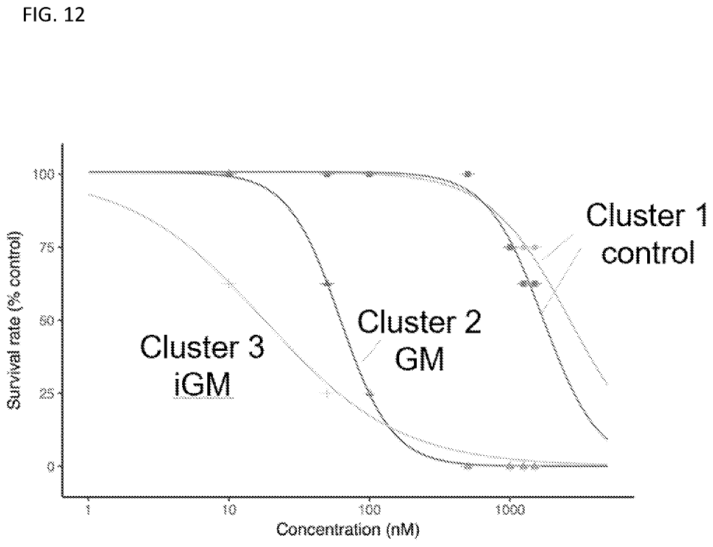

FIG. 13. Efficacy of Single Agent In Crohn's Library Xenograft.

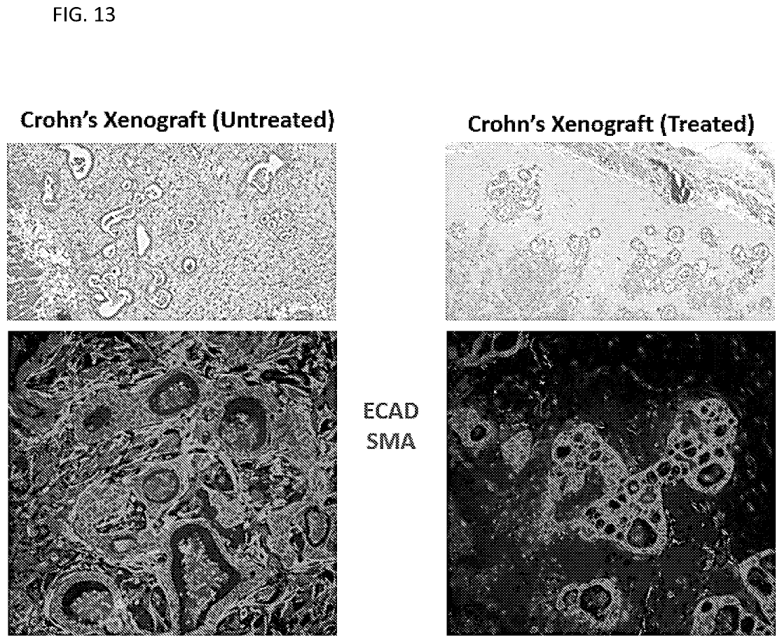

FIG. 14. Drug Combination Selective for Crohn's Pathogenic Stem Cell Relative to normal Terminal Ileum.

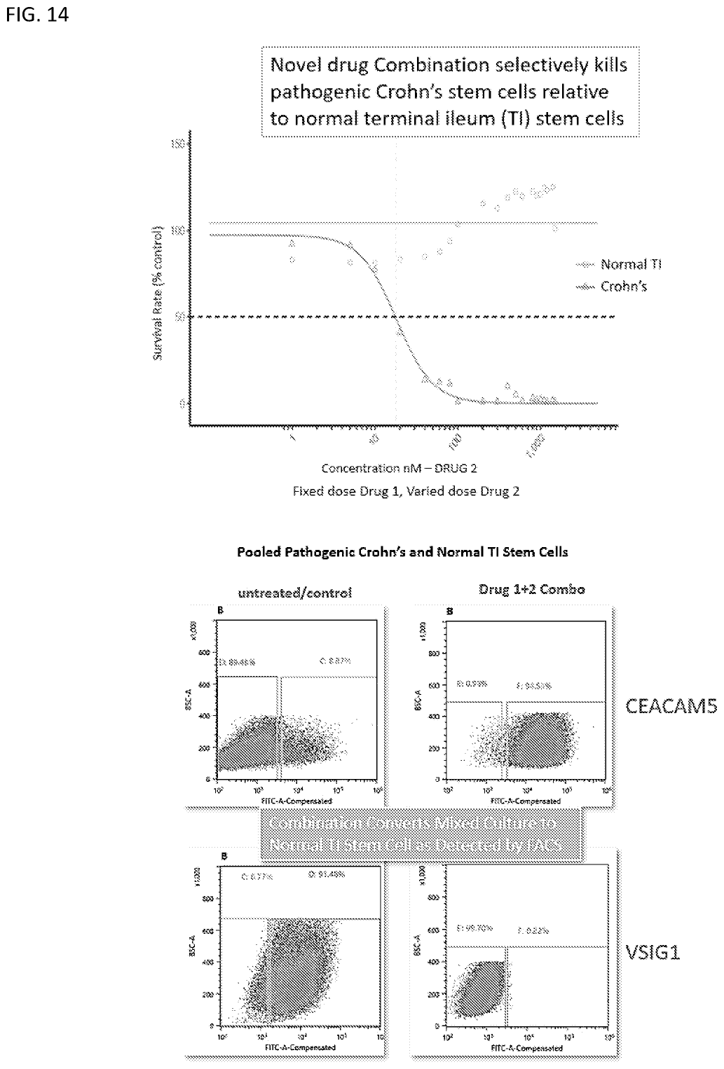

### DESCRIPTION OF ILLUSTRATIVE EMBODIMENTS

### I. Overview

Crohn's disease is an inflammatory bowel disorder marked by transmural lesions that frequently progress to strictures, fistulas, or perforations requiring repeated surgical intervention. While its onset is typically in young adults, 15 percent of cases arise in children who tend to have severe and extensive disease, frequent need for corticosteroids and immunosuppression, and enhanced risk for colorectal cancer. Though immunosuppressants and anti-inflammatory biologics can slow the progression of Crohn's disease, it is not clear that they have lessened the need of surgical intervention, an impasse that has fueled the search for therapeutic targets more proximal to the disease. This search is complicated by the large environmental contribution to this disease reflected by the low concordance among monozygotic twins, and by the polygenic nature of the remaining, inherited risk. Nevertheless, genome-wide association studies (GWAS) and pathophysiology are beginning to define the underlying genetic structure and biology of Crohn's disease. In particular, there is a stunning overlap of risk loci for Crohn's and mycobacterial infections, and many of the 170 loci discovered to date implicate genes of adaptive and innate immune processes that are likely involved in the containment of gut microbes. Consistent with this emerging “barrier defect” hypothesis are deficiencies in antimicrobial functions of Paneth cells in Crohn's disease patients, defective autophagy processing of microbial antigens by mucosal epithelial cells and altered responsiveness of mucosal immune cells. Despite these emerging data on mucosal barrier abnormalities in Crohn's, it remains unclear whether they are primary events or secondary consequences of the inflammatory state of this disease. It is also unclear how defective barrier function might explain the alternate regional presentations of Crohn's, its skip-lesion patterning, or the high rates of recurrence following ileo-colonic resection.

Most approaches to the treatment of Crohn's disease, Ulcerative Colitis and other forms of Inflammatory Bowel Diseases (IBD) focus on reducing or inhibiting the inflammatory components of these diseases. However, as described here, the inflammatory symptoms of Crohn's and other forms of IBD are a consequence of an altered epithelial lining generated by an epigenetically distinct set of stem cells that become activated in the tissue—with inflammation being caused by the altered epithelia. As described in greater detail below and the attached figures, in the case of inflammatory bowel diseases such as Crohn's, the inventors have found an epigenetic distinct set of stem cells of the terminal ileum—where the inflammatory storm that characterizes this disease occurs. In this case, the stem cells that give rise to the lining of the terminal ileum are altered in a way that cause them to give rise to an epithelial lining that is similar to what occurs further up the digestive tract where absorption of nutrients occur and is not serving as a barrier to bacteria the way the terminal ileum should. In addition, this epigenetic variation to these otherwise minor populations of stem cells is also turning on genes which attract immune cells, such as a signals and activators of the innate and/or adaptive immune systems. Whatever the initial insult is that causes this shift, the immune response in the gut is perpetuated by this altered epithelial lining produced by these stem cells.

The present disclosure addresses IBD from the standpoint of mucosal stem cells cloned from defined regions of the gastrointestinal tract. In the case of both pediatric and adult Crohn's disease, for example, isolation of those stem cells according to the methods of the present disclosure reveals a pattern of inflammatory gene expression in stem cells from the terminal ileum and colon that is epigenetically maintained despite months of continuous cultivation in the absence of immune or stromal cells, or of intestinal microbes. Superimposed on this distributed inflammatory phenotype is a differentiation defect that profoundly and specifically alters the mucosal barrier properties of the terminal ileum. And while the immediate basis of this barrier defect can be traced to a loss of ATOH1, a transcription factor required for secretory cell differentiation in the colon, this repression of ATOH1 is only emblematic of a more profound alteration of the terminal ileum in Crohn's disease involving a homeotic transformation of stem cells to a developmental ground state represented by the duodenum and jejunum. Lastly, the co-existence of diseased and normal stem cells within the same endoscopic biopsies of Crohn's disease patients implicates an epigenetically enforced heterogeneity among mucosal stem cells in the dynamics of this condition.

### II. Definitions

“Inflammatory bowel disease”, or “IBD”, is a term that encompasses both ulcerative colitis (inflammation of the lining of the large intestine) and Crohn's disease (inflammation of the lining and wall of the large and/or small intestine). When inflamed, the lining of the intestinal wall is red and swollen, becomes ulcerated, and bleeds. Although lesions associated with IBD can heal by themselves, most are recurrent. Chronic lesions occur in individuals with underlying diseases of various types whose medical conditions compromise the body's ability to repair injured tissue on its own (e.g., diabetes).

One type of lesion associated with IBD is an ulcer. A lesion is an open sore, an abrasion, a blister, or a shallow crater resulting from the sloughing or erosion of the top layer of epithelial cells and, sometimes, subcutaneous tissues. Although an ulcer can technically occur anywhere on the skin (e.g., a wound), the term “ulcer”, which is used loosely and interchangeably with “gastric ulcer” and “peptic ulcer”, usually refers to disorders in the upper digestive tract.

The term “an aberrant expression”, as applied to a nucleic acid of the present disclosure, refers to level of expression of that nucleic acid which differs from the level of expression of that nucleic acid in healthy gastroinstestinal tissue, or which differs from the activity of the polypeptide present in a healthy subject. An activity of a polypeptide can be aberrant because it is stronger than the activity of its native counterpart. Alternatively, an activity can be aberrant because it is weaker or absent relative to the activity of its native counterpart. An aberrant activity can also be a change in the activity; for example, an aberrant polypeptide can interact with a different target peptide. A cell can have an aberrant expression level of a gene due to overexpression or underexpression of that gene.

“Amino acid sequence” as used herein refers to an oligopeptide, peptide, polypeptide, or protein sequence, and fragment thereof, and to naturally occurring or synthetic molecules. Fragments of an expression product of an IBD gene sequence (an “IBD gene product”) are preferably about 5 to about 15 amino acids in length and retain the biological activity or the immunological activity of an IBD gene product. Where “amino acid sequence” is recited herein to refer to an amino acid sequence of a naturally occurring protein molecule, amino acid sequence, and like terms, are not meant to limit the amino acid sequence to the complete, native amino acid sequence associated with the recited protein molecule.

The term “antibody” broadly refers to any immunoglobulin (Ig) molecule and immunologically active portions of immunoglobulin molecules (i.e., molecules that contain an antigen binding site that immunospecifically bind an antigen) comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Nonlimiting embodiments of which are discussed below, and include but are not limited to a variety of forms, including full length antibodies and antigen-binding portions thereof; including, for example, an immunoglobulin molecule, a monoclonal antibody, a chimeric antibody, a CDR-grafted antibody, a human antibody, a humanized antibody, a single chain antibody, a Fab, a F(ab′), a F(ab′)2, a Fv antibody, fragments produced by a Fab expression library, a disulfide linked Fv, a scFv, a single domain antibody (dAb), a diabody, a multispecific antibody, a dual specific antibody, an anti-idiotypic antibody, a bispecific antibody, a functionally active epitope-binding fragment thereof, bifunctional hybrid antibodies (e.g., Lanzavecchia et al., Eur. J. Immunol. 17, 105 (1987)) and single chains (e.g., Huston et al., Proc. Natl. Acad. Sci. U.S.A., 85, 5879-5883 (1988) and Bird et al., Science 242, 423-426 (1988), which are incorporated herein by reference) and/or antigen-binding fragments of any of the above (See, generally, Hood et al., Immunology, Benjamin, N.Y., 2ND ed. (1984), Harlow and Lane, Antibodies. A Laboratory Manual, Cold Spring Harbor Laboratory (1988) and Hunkapiller and Hood, Nature, 323, 15-16 (1986), which are incorporated herein by reference). Antibodies also refer to immunoglobulin molecules and immunologically active portions of immunoglobulin molecules, i.e., molecules that contain antigen or target binding sites or “antigen-binding fragments.” The antibody or immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art. Furthermore, in humans, the light chain can be a kappa chain or a lambda chain.

The term “specific affinity binder” refers to an antibody as well as to a non-antibody protein scaffold i.e., smaller proteins that are capable of achieving comparable affinity and specificity using molecular structures that can be for example one-fifth to one-tenth the size of full antibodies, and also to nucleic acid aptamers. In some embodiments, the specific affinity binder of the present disclosure is a non-antibody polypeptide. In some embodiments, the non-antibody polypeptide can include but is not limited to peptibodies, DARPins, avimers, adnectins, anticalins, affibodies, affilins, atrimers, bicyclic peptides, centryins, Cys-knots, Fynomers, Kunitz domains, Obodies, pronectins, Tn3, maxibodies, or other protein structural scaffold, or a combination thereof.

A disease, disorder, or condition “associated with” or “characterized by” an aberrant expression of an IBD gene sequence refers to a disease, disorder, or condition in a subject which is caused by, contributed to by, or causative of an aberrant level of expression of a nucleic acid.

“Biological activity” or “bioactivity” or “activity” or “biological function”, which are used interchangeably, herein mean an effector or antigenic function that is directly or indirectly performed by a polypeptide (whether in its native or denatured conformation), or by any subsequence thereof. Biological activities include binding to polypeptides, binding to other proteins or molecules, activity as a DNA binding protein, as a transcription regulator, ability to bind damaged DNA, etc. A bioactivity can be modulated by directly affecting the subject polypeptide. Alternatively, a bioactivity can be altered by modulating the level of the polypeptide, such as by modulating expression of the corresponding gene.

The terms “complementary” or “complementarity”, as used herein, refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in amplification reactions, which depend upon binding between nucleic acids strands and in the design and use of PNA molecules.

A “composition comprising a given polynucleotide sequence” as used herein refers broadly to any composition containing the given polynucleotide sequence. The composition may comprise a dry formulation or an aqueous solution. Compositions comprising polynucleotide sequences encoding an IBD gene product or fragments thereof may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., SDS) and other components (e.g., Denhardt's solution, dry milk, salmon sperm DNA, etc.).

The term “correlates with expression of a polynucleotide”, as used herein, indicates that the detection of the presence of ribonucleic acid that is similar to one of IBD genes by northern analysis is indicative of the presence of mRNA encoding an IBD gene product in a sample and thereby correlates with expression of the transcript from the polynucleotide encoding the protein.

A “deletion”, as used herein, refers to a change in the amino acid or nucleotide sequence and results in the absence of one or more amino acid residues or nucleotides.

As is well known, genes or a particular polypeptide may exist in single or multiple copies within the genome of an individual. Such duplicate genes may be identical or may have certain modifications, including nucleotide substitutions, additions or deletions, which all still code for polypeptides having substantially the same activity. The term “DNA sequence encoding an IBD polypeptide” may thus refer to one or more genes within a particular individual. Moreover, certain differences in nucleotide sequences may exist between individual organisms, which are called alleles. Such allelic differences may or may not result in differences in amino acid sequence of the encoded polypeptide yet still encode a polypeptide with the same biological activity.

As used herein, the terms “gene”, “recombinant gene”, and “gene construct” refer to a nucleic acid of the present disclosure associated with an open reading frame, including both exon and (optionally) intron sequences.

A “recombinant gene” refers to nucleic acid encoding a polypeptide and comprising exon sequences, though it may optionally include intron sequences which are derived from, for example, a related or unrelated chromosomal gene. The term “intron” refers to a DNA sequence present in a given gene which is not translated into protein and is generally found between exons.

The term “growth” or “growth state” of a cell refers to the proliferative state of a cell as well as to its differentiative state. Accordingly, the term refers to the phase of the cell cycle in which the cell is, e.g., GO, G1, G2, prophase, metaphase, or telophase, as well as to its state of differentiation, e.g., undifferentiated, partially differentiated, or fully differentiated.

Without wanting to be limited, differentiation of a cell is usually accompanied by a decrease in the proliferative rate of a cell.

“Homology” or “identity” or “similarity” refers to sequence similarity between two peptides or between two nucleic acid molecules, with identity being a more strict comparison. Homology and identity can each be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e., structurally related, at positions shared by the amino acid sequences. An “unrelated” or “non-homologous” sequence shares less than 40% identity, though preferably less than 25% identity, with one of the sequences of the present disclosure.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

Databases with individual sequences are described in Methods in Enzymology, ed. Doolittle, supra. Databases include Genbank, EMBL, and DNA Database of Japan (DDBJ).

The term “hybridization”, as used herein, refers to any process by which a strand of nucleic acid binds with a complementary strand through base pairing.

An “insertion” or “addition”, as used herein, refers to a change in an amino acid or nucleotide sequence resulting in the addition of one or more amino acid residues or nucleotides, respectively, as compared to the naturally occurring molecule.

The term “isolated” as used herein with respect to nucleic acids, such as DNA or RNA, refers to molecules separated from other DNAs, or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

“Microarray” refers to an array of distinct polynucleotides or oligonucleotides synthesized on a substrate, such as paper, nylon or other type of membrane, filter, chip, glass slide, or any other suitable solid support.

The terms “modulated” and “differentially regulated” as used herein refer to both upregulation (i.e., activation or stimulation (e.g., by agonizing or potentiating)) and downregulation (i.e., inhibition or suppression (e.g., by antagonizing, decreasing or inhibiting)).

The term “mutated gene” refers to an allelic form of a gene, which is capable of altering the phenotype of a subject having the mutated gene relative to a subject which does not have the mutated gene. If a subject must be homozygous for this mutation to have an altered phenotype, the mutation is said to be recessive. If one copy of the mutated gene is sufficient to alter the genotype of the subject, the mutation is said to be dominant. If a subject has one copy of the mutated gene and has a phenotype that is intermediate between that of a homozygous and that of a heterozygous subject (for that gene), the mutation is said to be co-dominant.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

As used herein, “gene silencing” or “gene silenced” in reference to an activity of an RNAi molecule, for example a siRNA or miRNA refers to a decrease in the mRNA level in a cell for a target gene (i.e., an IBD gene sequence) by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of downstream processing of the RNA (i.e., although siRNAs are believed to have a specific method of in vivo processing resulting in the cleavage of mRNA, such sequences can be incorporated into the vectors in the context of the flanking sequences described herein).

The term “RNAi” can include both gene silencing RNAi molecules, and also RNAi effector molecules which activate the expression of a gene. By way of an example only, in some embodiments RNAi agents which serve to inhibit or gene silence are useful in the methods, kits and compositions disclosed herein to alter the expression of, such as in particular inhibit the expression of an IBD gene sequence.

As used herein, a “siRNA” refers to a nucleic acid that forms a double stranded RNA, which double stranded RNA has the ability to reduce or inhibit expression of a target IBD gene sequence when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

As used herein, the term “promoter” means a DNA sequence that regulates expression of a selected DNA sequence operably linked to the promoter, and which effects expression of the selected DNA sequence in cells. The term encompasses “tissue specific” promoters, i.e., promoters which effect expression of the selected DNA sequence only in specific cells (e.g., cells of a specific tissue). The term also covers so-called “leaky” promoters, which regulate expression of a selected DNA primarily in one tissue, but cause expression in other tissues as well. The term also encompasses non-tissue specific promoters and promoters that constitutively expressed or that are inducible (i.e., expression levels can be controlled).

The terms “protein”, “polypeptide”, and “peptide” are used interchangeably herein when referring to a gene product.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays of the disclosure to identify compounds that modulate a bioactivity.

A “substitution”, as used herein, refers to the replacement of one or more amino acids or nucleotides by different amino acids or nucleotides, respectively.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of one of the genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring forms of the polypeptide.

As used herein, the term “transgene” means a nucleic acid sequence (or an antisense transcript thereto) which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extra-chromosomally replicating DNA. In the typical transgenic animals described herein, the transgene causes cells to express a recombinant form of one of the subject polypeptide, e.g., either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques.

As used herein, the terms “treatment” and “treating” refer to an approach for obtaining beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

A “therapeutic effect,” as used herein encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “subject” or “patient” as used herein refers to any animal, such as a mammal, for example a human. The methods and compositions described herein can be useful in both human therapeutics and veterinary applications. In some embodiments, the patient is a mammal, and in some embodiments, the patient is human. For veterinary purposes, the terms “subject” and “patient” include, but are not limited to, farm animals including cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

As used herein, “pharmaceutically acceptable salt thereof” includes an acid addition salt or a base salt.

As used herein, “pharmaceutically acceptable carrier” includes any material which, when combined with a compound of the disclosure, allows the compound to retain biological activity, such as the ability to induce apoptosis of leukemia or breast tumor cells, and is non-reactive with the subject's immune system. Examples include, but are not limited to, any of the standard pharmaceutical carriers such as a phosphate buffered saline solution, water, emulsions such as oil/water emulsions, and various types of wetting agents. Compositions comprising such carriers are formulated by well known conventional methods (see, for example, Remington's Pharmaceutical Sciences, Chapter 43, 14th Ed., Mack Publishing Co., Easton, Pa.).

### III. IBD Stem Cell Inhibitors

The inventors have observed that certain of these drug agents are able to selectively kill pathogenic stem cells isolated from gastrointestinal biopsies.

a. HSP90, HSP70 and dual HSP90/70 Inhibitors

For example, one aspect of the disclosure relates to the use of an HSP90 inhibitor, an HSP70 inhibitor or a combination thereof including in the form of a single molecule dual HSP90/70 inhibitor, as part of a treatment for IBD.

Examples of Hsp90 inhibitors include, but are not limited to, geldanamycin, radicicol, 17-N-allylamino-17-demethoxygeldanamycin (also known as tanespicmycin or 17-AAG) (BMS), herbimycin A, novobiocin sodium (U-6591), 17-GMB-APA-GA, 17-AAG-nab, 17-AEP, macbecin I, CCT 018159, gedunin, PU24FC1, PU-H71, PU-DZ8, PU3, NVP-AUY922 (Novartis), NVP-HSP990 (Novartis), retaspimycin hydrochloride/IPI-504 (Infinity), BIIB021/CNF2024 (Biogen Idec), ganetespib (STA-9090, Synta), STA-1474, SNX-5422/mesylate (Pfizer), BIIB028 (Biogen Idec), KW-2478 (Kyowa Hakko Kirin), AT13387 (Astex), XL888 (Exelixis), MPC-3100 (Myriad), ABI-010/nab (nanoparticle, albumin bound)-17AAG (Abraxis), 17-aminodemethoxygeldanamycin (IPI-493), 17-dimethylaminoethylamino-17-demethoxygeldanamycin (17-DMAG), SNX-2112, SNX-7081, Debio0932, B11B021, MPC-3100, MPC-0767, PU3, PU-H58, DS-2248, CCT018159, CCT0129397, BJ-B11, elesclomol (STA-4783), G3130, a herbimycin (such as Herbimycin A; Herbimycin B; Herbimycin C), radester, KNK437, HSP990, NVP-BEP800, Celastrol, Alvespimycin, Autolytimycin, AUY13387, BX-2819, CUDC-305, Curvularin, Flavopiridol, Lebstatin, L-783,277, LL-Z1640-2, Maytansine, MPC-6827, Mycograb, NCS-683664, NXD30001, PF-04929113, Pochonin D, Reblastatin, Redicicol, Rifabutin, VER49009, Xestodccalactone, and Zearalenone.

In certain embodiments, the HSP90 inhibitor is selected from the group consisting of 17-AAG, 17-AEP, 17-DMAG, B11B021, CCT018159, Celastrol, Gedunin, NVP-AUY922 (aka AUY922), PU-H71, and Radicicol.

In certain embodiments, the HSP90 Inhibitor is a benzoquinone class of compounds known as ansamycins (e.g., herbimycin A, geldanamycin, 17-AAG, macbecin, and ansatrienins). These include:

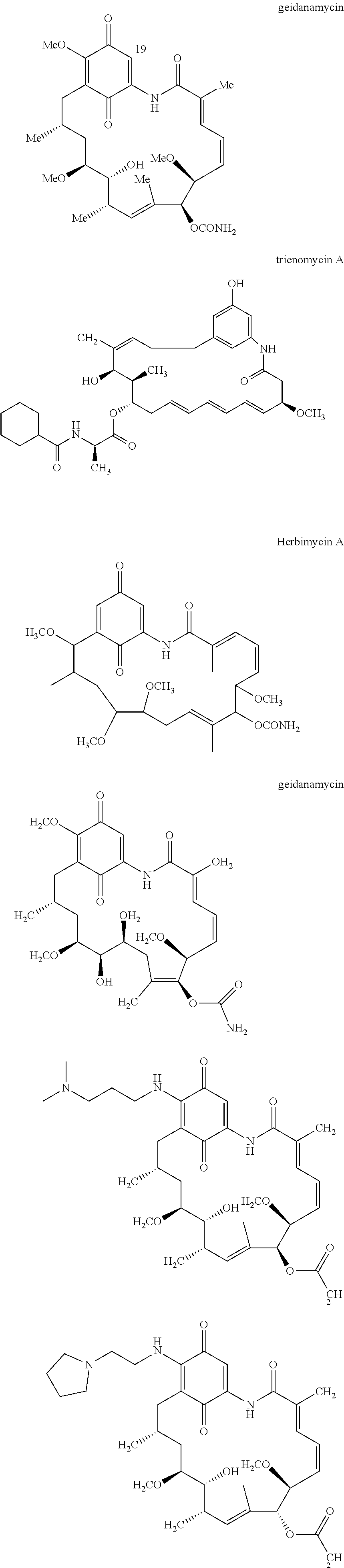

In certain embodiments, the HSP90 Inhibitor is a benzoyl compound represented by formula:

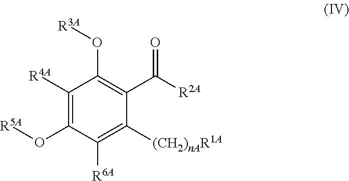

wherein
nA represents an integer of 1 to 5;
R1A represents substituted or unsubstituted lower alkyl, substituted or unsubstituted lower alkoxy, substituted or unsubstituted cycloalkyl, substituted or unsubstituted lower alkoxycarbonyl, substituted or unsubstituted heterocycle-alkyl, substituted or unsubstituted aryl, —C(═O)N(R7A)(R8A) (wherein R7A and R8A may be the same or different, and each represents a hydrogen atom, substituted or unsubstituted lower alkyl, substituted or unsubstituted cycloalkyl, substituted or unsubstituted lower alkanoyl, substituted or unsubstituted aryl, a substituted or unsubstituted heterocyclic group, substituted or unsubstituted aralkyl, substituted or unsubstituted heterocycle-alkyl, or substituted or unsubstituted aroyl, or R7A and R8A are combined together with the adjacent nitrogen atom thereto to form a substituted or unsubstituted heterocyclic group), or —N(R9A)(R10A) (wherein R9A and R10A have the same meanings as the above R7A and R8A, respectively);
R2A represents substituted or unsubstituted aryl or a substituted or unsubstituted aromatic heterocyclic group;
R3A and R5A may be the same or different, and each represents a hydrogen atom, substituted or unsubstituted lower alkyl, substituted or unsubstituted lower alkenyl, substituted or unsubstituted lower alkanoyl, substituted or unsubstituted cycloalkyl, substituted or unsubstituted aralkyl, or substituted or unsubstituted aroyl;
R4A represents a hydrogen atom, hydroxy, or halogen; and
R6A represents a hydrogen atom, halogen, cyano, nitro, substituted or unsubstituted lower alkyl, substituted or unsubstituted lower alkenyl, substituted or unsubstituted lower alkynyl, substituted or unsubstituted lower alkoxy, substituted or unsubstituted cycloalkyl, amino, lower alkylamino, di(lower alkyl)amino, carboxy, substituted or unsubstituted lower alkoxycarbonyl, substituted or unsubstituted lower alkanoyl, substituted or unsubstituted aryloxy, substituted or unsubstituted aryl, a substituted or unsubstituted heterocyclic group, substituted or unsubstituted aralkyl, or substituted or unsubstituted heterocycle-alkyl;
   or is a prodrug thereof; or a pharmaceutically acceptable salt thereof, and the like.

In certain embodiments, the HSP90 Inhibitor is a benzene derivative represented by formula:

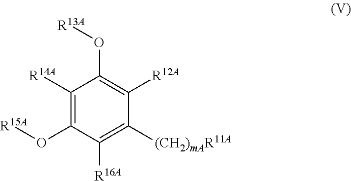

wherein
mA represents an integer of 0 to 10;
R11A represents a hydrogen atom, hydroxy, cyano, carboxy, nitro, halogen, substituted or unsubstituted lower alkyl, substituted or unsubstituted lower alkenyl, substituted or unsubstituted lower alkynyl, substituted or unsubstituted cycloalkyl, substituted or unsubstituted lower alkoxycarbonyl, substituted or unsubstituted aroyl, substituted or unsubstituted lower alkanoyl, substituted or unsubstituted heterocycle-alkyl, substituted or unsubstituted aryl, substituted or unsubstituted aralkyl, substituted or unsubstituted arylsulfonyl, a substituted or unsubstituted heterocyclic group, —C(═O)N(R17A)(R18A) (wherein R17A and R18A may be the same or different, and each represents a hydrogen atom, substituted or unsubstituted lower alkyl, substituted or unsubstituted cycloalkyl, substituted or unsubstituted lower alkanoyl, substituted or unsubstituted aryl, a substituted or unsubstituted heterocyclic group, substituted or unsubstituted aralkyl, substituted or unsubstituted heterocycle-alkyl, or substituted or unsubstituted aroyl, or R17A and R18A are combined together with the adjacent nitrogen atom thereto to form a substituted or unsubstituted heterocyclic group), or —N(R19A)(R20A) (wherein R19A and R20A may be the same or different, and each represents a hydrogen atom, substituted or unsubstituted lower alkylsulfonyl, substituted or unsubstituted lower alkyl, substituted or unsubstituted cycloalkyl, substituted or unsubstituted lower alkanoyl, substituted or unsubstituted aryl, a substituted or unsubstituted heterocyclic group, substituted or unsubstituted aralkyl, substituted or unsubstituted heterocycle-alkyl, substituted or unsubstituted aroyl, or R19A and R20A are combined together with the adjacent nitrogen atom thereto to form a substituted or unsubstituted heterocyclic group), or —C(═O)N(R21A)(R22A) (wherein R21A and R22A have the same meanings as R17 and R18 defined above, respectively, or R21A and R22A are combined together with the adjacent nitrogen atom thereto to form a substituted or unsubstituted heterocyclic group) or —OR23A (wherein R23A represents substituted or unsubstituted lower alkyl, substituted or unsubstituted lower alkenyl, substituted or unsubstituted lower alkanoyl, substituted or unsubstituted aryl, a substituted or unsubstituted heterocyclic group, substituted or unsubstituted aralkyl, or substituted or unsubstituted heterocycle-alkyl);
R12A represents substituted or unsubstituted lower alkyl, substituted or unsubstituted lower alkenyl, substituted or unsubstituted lower alkynyl, substituted or unsubstituted aryl or a substituted or unsubstituted heterocyclic group;
R13A and R15A may be the same or different, and each represents a hydrogen atom, substituted or unsubstituted lower alkyl, substituted or unsubstituted lower alkenyl, substituted or unsubstituted lower alkanoyl, substituted or unsubstituted cycloalkyl, substituted or unsubstituted lower alkylsulfonyl, substituted or unsubstituted arylsulfonyl, carbamoyl, sulfamoyl, substituted or unsubstituted lower alkylaminocarbonyl, substituted or unsubstituted di(lower alkyl)aminocarbonyl, substituted or unsubstituted lower alkoxycarbonyl, substituted or unsubstituted heterocycle-carbonyl, substituted or unsubstituted aralkyl, or substituted or unsubstituted aroyl;
R14A and R16A may be the same or different, and each represents a hydrogen atom, hydroxy, halogen, cyano, nitro, substituted or unsubstituted lower alkyl, substituted or unsubstituted lower alkenyl, substituted or unsubstituted lower alkynyl, substituted or unsubstituted lower alkoxy, substituted or unsubstituted cycloalkyl, amino, lower alkylamino, di(lower alkyl)amino, carboxy, substituted or unsubstituted lower alkoxycarbonyl, substituted or unsubstituted aryloxy, substituted or unsubstituted aryl, a substituted or unsubstituted heterocyclic group, substituted or unsubstituted lower alkanoyl, substituted or unsubstituted aralkyl, or substituted or unsubstituted heterocycle-alkyl),
   or is a prodrug thereof; or a pharmaceutically acceptable salt thereof, and the like.

Radicicol, a macrocyclic lactone antibiotic, has been shown to inhibit the function of HSP90. To further investigate the biological mechanism of radicicol and its analogs in regulating HSP90 and establish the fundamental structure-activity relationship, a number of radicicol analogs have been synthesized and studied. The term “radicicol analogs” or “radicicol derivatives” as used herein denotes macrocyclic lactone compounds that are structurally similar to radicicol. Specifically, the “radicicol analogs” or “radicicol derivatives” refer to compounds of fused bicyclic ring structure wherein a six-membered aromatic ring shares two carbon atoms with a 12- to 16-membered non-aromatic ring containing a lactone group and at least one olefin group in the core of the 12- to 16-membered ring. The radicicol analogs/derivatives may have one or more substituents on the six-membered aromatic ring or the 12- to 16-membered non-aromatic ring. It is noted that the terms “analog” and “derivative” are used interchangeably in the present application. A number of radicicol analogs have been disclosed in patent publications including WO 96/33989, WO 98/18780, WO 99/55689, U.S. Pat. Nos. 7,115,651, 5,731,343, and 5,077,165, all of which are herein incorporated by reference in their entirety.

It has been reported that certain purine scaffold-based compounds are HSP90 inhibitors. (See, for example, WO 02/36705, WO 03/037860, and WO 2006/084030, all of which are herein incorporated by reference in their entirety) These purine scaffold-based HSP90 inhibitors typically have a structure wherein an adenine ring and a six-membered aryl or heteroaryl ring are linked through a linker which can be methylene, fluorinated methylene, sulfur, oxygen, nitrogen, carbonyl, imine, sulfinyl, or sulfonyl. PU3 and PU24FCI are examples of two compounds exemplifying the purine scaffold-based HSP90 inhibitors.

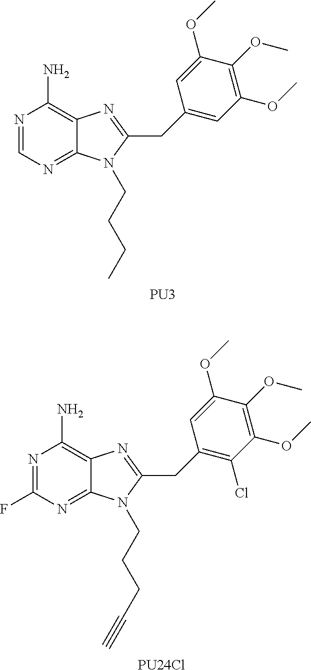

Some pyrazole or imidazole scaffold-based compounds are known to inhibit HSP90. These pyrazole or imidazole scaffold-based HSP90 inhibitors are typically non-fused tricyclic compounds wherein two aryl or heteroaryl rings are attached to two adjacent positions (carbon or nitrogen atom) of a pyrazole or imidazole ring, respectively. (See, for example, WO 2007/021877, which is herein incorporated by reference in its entirety, or Vernalis Ltd, Bioorg Med Chem Lett, 2006, 16, 2543-2548, or Sharp et al., Molecular Cancer Therapeutics, 2007, 6, 1 198-121 1). Examples of pyrazole or imidazole scaffold-based HSP90 inhibitors include:

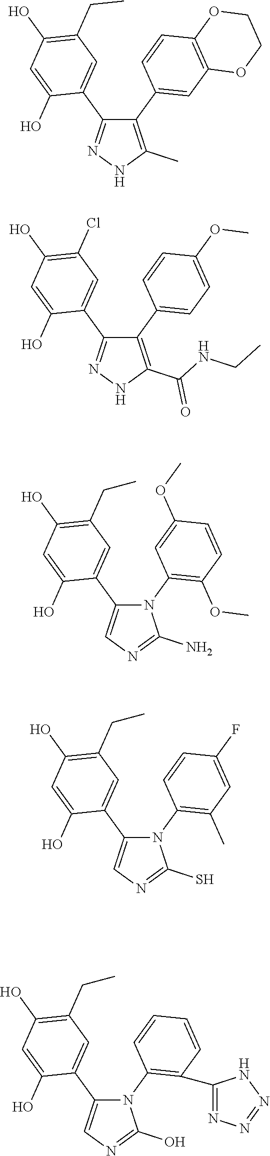 [SMILES: CCc1cc(-c2n[nH]c(C)c2-c2ccc3c(c2)OCCO3)c(O)cc1O ; CCNC(=O)c1[nH]nc(-c2cc(Cl)c(O)cc2O)c1-c1ccc(OC)cc1 ; CCc1cc(-c2cnc(N)n2-c2cc(OC)ccc2OC)c(O)cc1O ; CCc1cc(-c2cnc(S)n2-c2ccc(F)cc2C)c(O)cc1O ; CCc1cc(-c2cnc(O)n2-c2ccccc2-c2nnn[nH]2)c(O)cc1O]

Another class of HSP90 inhibitors are tetrahydroindolone and tetrahydroindazolone derivatives reported in WO 2006/091963, the disclosure of which is herein incorporated by reference in its entirety. These tetrahydroindolone or tetrahydroindazolone based HSP90 inhibitors generally have a structure wherein a substituted aryl group is directly attached to the nitrogen atom of a tetrahydroindolone or tetrahydroindazolone. Examples in WO 2006/091963 include:

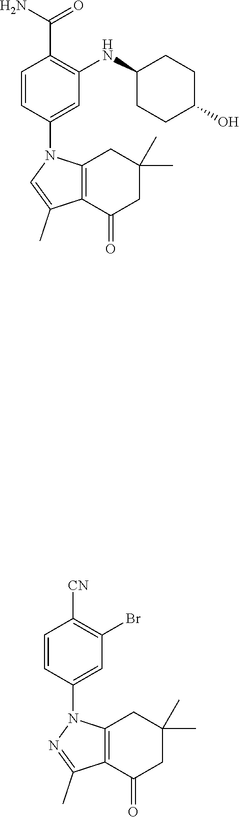 [SMILES: Cc1cn(-c2ccc(C(N)=O)c(N[C@H]3CC[C@H](O)CC3)c2)c2c1C(=O)CC(C)(C)C2 ; Cc1nn(-c2ccc(C#N)c(Br)c2)c2c1C(=O)CC(C)(C)C2]

A number of HSP90 inhibitors in various compound classes have been developed as potential agents for cancer treatment. These include purine-based compounds (PCT publications WO/2006/084030; WO/2002/036075; U.S. Pat. No. 7,138,401; US20050049263; Biamonte et al., 2006, J. Med. Chem. 49:817-828; Chiosis, 2006, Curr. Top. Med. Chem. 6:1 183-1 191; He et al., 2006, J. Med. Chem. 49:381-390), pyrazole-based compounds (Rowlands et al., 2004, Anal. Biochem. 327:176-183; Dymock et al., 2005, J. Med. Chem. 48:4212-4215; PCT publication WO/2007/021966; WO/2006/039977; WO/2004/096212; WO/2004/056782; WO/2004/050087; WO/2003/055860; U.S. Pat. No. 7,148,228), peptidomimetic shepherdin (Plescia et al., 2005, Cancer Cell 7:457-468; US publication 20060035837), and HSP90 inhibitors in other compound classes (PCT publications WO/2006/123165; WO/2006/109085; WO/2005/028434; U.S. Pat. Nos. 7,160,885; 7,138,402; 7,129,244; US20050256183; US20060167070; US20060223797; WO2006091963).

In certain embodiments, the anti-PESC agent is an HSP90 inhibitor is a compound selected from the group:
geldamycin;
17-AAG (17-allyl-17-demethoxygeldanamycin);
17-DMAG (17-desmethoxy-17-N,N-dimethylaminoethylaminogel danamycin); IPI-504 (17-allylamino-I 7-demethoxygeldanamycin hydroquinone hydrochloride); IP 1-493 (17-desmethoxy-17-amino geldanamycin);
BIIB021 ([6-Chloro-9-(4-methoxy-3,5-dimethylpyridin-2-ylmethyl)-9H-purin-2-yl]amine);
MPC-3100 ((S)—I-(4-(2-(6-amino-8-((6-bromobenzo[d][I,3]dioxol-5-yl)thio)-9H-purin-9-yl)ethyl)piperidin-1-yl)-2-hydroxypropan-1-one);
Debio 0932 (2-((6-(dimethylamino)benzo [d] [1,3]dioxol-5-yl)thio)-1-(2-(neopentylamino)ethyl)-IH-imidazo[4,5-c]pyridin-4-amine);
PU-H71 (6-Amino-8-[(6-iodo-I,3-benzodioxol-5-yl)thio]-N—(I-methylethyl)-9H-purine-9-propanamine);
STA-9090 (5-[2,4-dihydroxy-5-(I-methylethyl)phenyl]-4-(I-methyl-IH-indol-5-yl)-2,4-dihydro-3H-1,2,4-triazol-3-one);
VER52296 (5-(2,4-Dihydroxy-5-isopropylphenyl)-N-ethyl-4-(4-(morpholinomethyl)phenyl)isoxazole-3-carboxamide);
KW-2478 (2-(2-ethyl-3,5-dihydroxy-6-(3-methoxy-4-(2-morpholinoethoxy)benzoyl)phenyl)-N,N-bis(2-methoxyethyl)acetamide); AT-13387 ((2,4-dihydroxy-5-isopropylphenyl)(5-((4-methylpiperazin-1-yl)methyl) iso indolin-2-yl)methanone);
Radicicol ((1 aR,2Z,4E, 14R, 15aR)-8-Chloro-I a, 14, 15, 15a-tetrahydro-9,11-dihydroxy-14-methyl-6H-oxireno[e][2]benzoxacyclotetradecin-6, 12(7H)-dione);
and
Celastrol ((9β, 13 a, 14β,20α)-3-Hydroxy-9, 13-dimethyl-2-oxo-24,25,26-trinoroleana-I(10),3,5,7-tetraen-29-oic acid);
or is a combination thereof, or a pharmaceutically acceptable salt thereof.

Exemplary HSP70 inhibitors include, but are not limited to, MKT-077 (1-Ethyl-2-[[3-ethyl-5-(3-methyl-2(3H)-benzothiazolylidene)-4-oxo-2-thiazolidinylidene]methyl]-pyridinium chloride), Omeprazole (5-Methoxy-2-[[(4-methoxy-3,5-dimethyl-2-pyridinyl)methyl]sulfinyl]-1H-benzimidazole), 5-(N,N-Dimethyl)amiloride hydrochloride (DMA), 2-phenylethynesulfonamide (PES), JG-98 (Li et al., ACS Med. Chem. Lett., (2013)4: 1042-1047), VER-155008, 2-phenylethynesulfonamide (PES), JG-98, 115-7c, apoptozole, JG-13, JG-48, MAL3-101, pifithrin-u, spergualin, YM-01, YM-08, VER15508 (5′-O-[(4-Cyanophenyl)methyl]-8-[[(3,4-dichlorophenyl)methyl]amino]-adenosine), Apoptozole (4-((2-(3,5-bis(trifluoromethyl)phenyl)-4,5-bis(4-methoxyphenyl)-1H-imidazol-1-yl)methyl)benzamide), HSP70-IN-1, JG2-38 ((2Z,5E)-5-(3,5-Dimethylbenzo[d]thiazol-2(3H)-ylidene)-3-ethyl-2-((3-((2-fluorophenyl)amino)pyridin-4-yl)methylene)thiazolidin-4-one). Additional HSP70 inhibitors are described in U.S. Pat. No. 9,642,843 and U.S. Patent Publication Nos. 2012/0252818, 2017/0014434, and 2018/0002325. See also, Taldone T, et al. Heat shock protein 70 inhibitors. 2,5′-thiodipyrimidines, 5-(phenylthio)pyrimidines, 2-(pyridin-3-ylthio)pyrimidines, and 3-(phenylthio)pyridines as reversible binders to an allosteric site on heat shock protein 70. J Med Chem. 2014 Feb. 27; 57(4):1208-24.

Exemplary HSP70 inhibitor structures include:

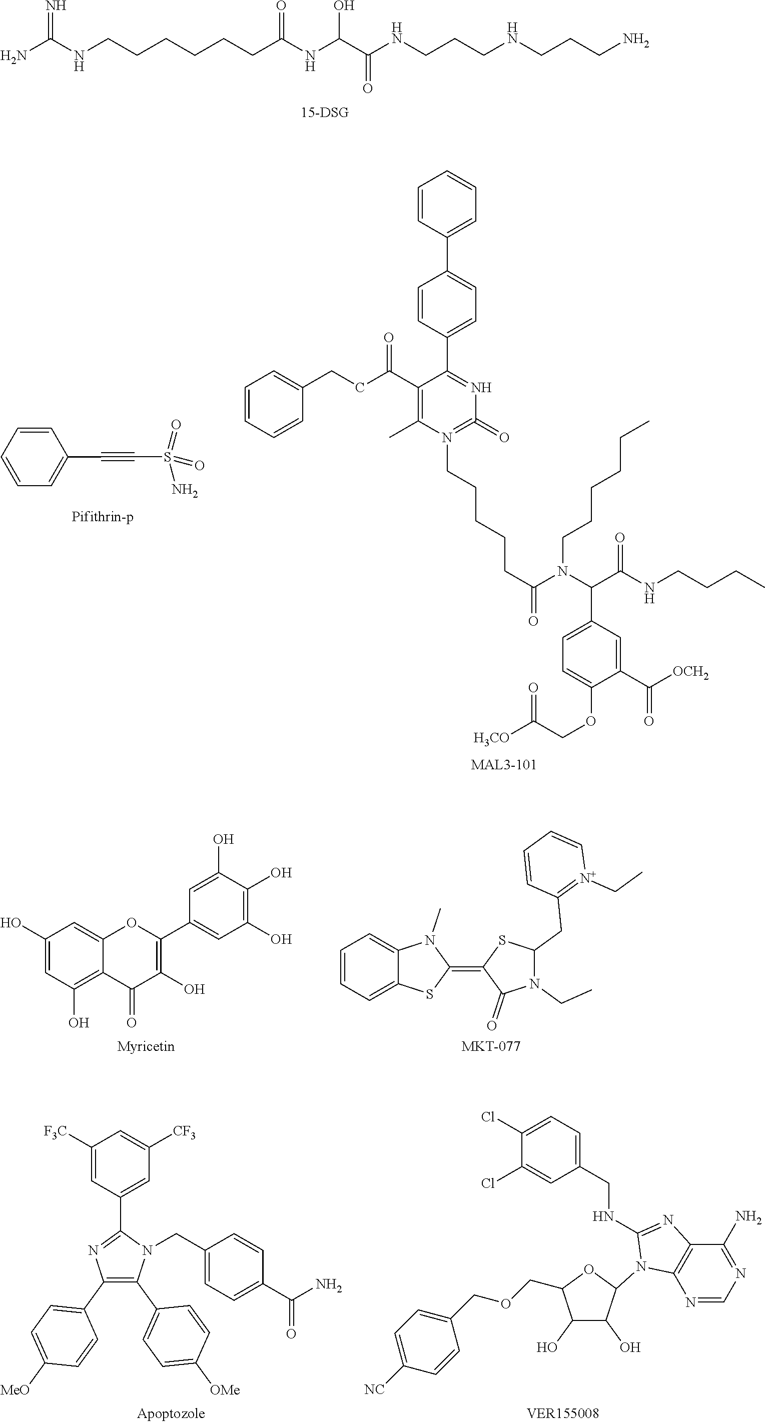

In certain embodiments the present disclosure provides compounds of formula I:

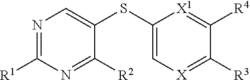

or a pharmaceutically acceptable salt thereof, wherein:
X is —N═ or —CH═;
X¹ is —N═ or —C(R⁵)—;
R⁵ is

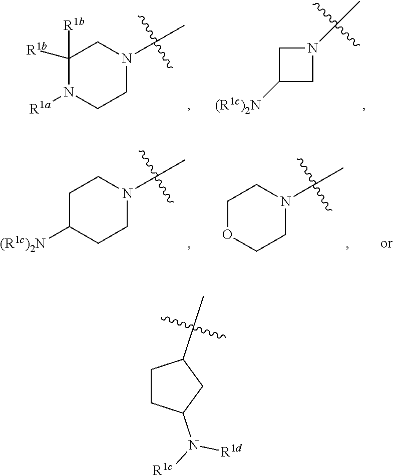

R^{1a} is

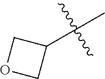 [SMILES: *C1COC1]

or C1-6 aliphatic optionally substituted with one or more groups independently selected from —OH, cyclopropyl, or 5-membered heteroaryl having 1-2 heteroatoms independently selected from nitrogen, oxygen or sulfur;
each R^{1b} is independently hydrogen, C1-4 alkyl, or two Rib groups are optionally taken together to form an oxo group;
each of R^{1C} and R^{1d} is independently hydrogen or C1-4 alkyl;
R² is —O—CH₂-Ring A, —NH—CH₂-Ring A, or —O—CH₂CH₂-Ring A;
   Ring A is unsubstituted phenyl, unsubstituted furanyl,

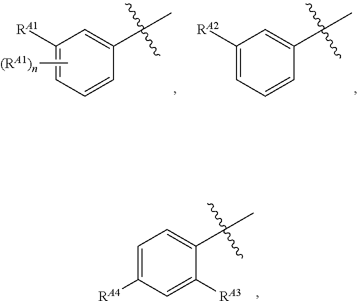

or pyridinyl optionally substituted with R^{A5};
each of R^{A1} is independently halogen, —CN, —C(═O)N(R)₂, —N(R)₂, —OR, —C(═O)R, —N₃, an optionally substituted 5- or 6-membered heterocyclyl or heteroaryl having one or two heteroatoms independently selected from nitrogen, oxygen, or sulfur, or C1-4 alkyl optionally substituted with one or more halogen;
each R is independently hydrogen or C1-4 alkyl optionally substituted with one or more halogen;
R^{A2} is —Cl, —Br, —I, —CN, —C(═O)N(R)₂, —N(R)₂, —OR, —C(═O)R, —N₃, an optionally substituted 5- or 6-membered heterocyclyl or heteroaryl having one or two heteroatoms independently selected from nitrogen, oxygen or sulfur, or C1-4 alkyl optionally substituted with one or more halogen;
n is 1 to 4;
R^{A3} is —H or —F;
R^{A4} is —F or —OR;
R^{A5} is —OR or —N(R)₂;
R³ is —C(O)N(R^{3a})₂, —OR b, —C(O)H, —C(O)OR, or —N(R^{3c})₂;
   each R^{3a} is independently hydrogen or Ci alkyl optionally substituted with one or more groups independently selected from halogen or 1-pyrrolidinyl;
   R^{3b} is hydrogen or C1-4 alkyl optionally substituted with one or more groups independently selected from halogen, C1-4 alkyl, C1-4 haloalkyl, oxo, or —N(R)₂;
   each R^{3c} is independently hydrogen or C1-4 alkyl optionally substituted with one or more groups independently selected from halogen, C1-4 alkyl, C1-4 haloalkyl, oxo, or —N(R)₂;
R⁴ is R, halogen, or —N(R)₂; and
R⁵ is hydrogen, methyl or —N(R)₂.

In certain embodiments, the anti-PESC agent is an HSP70 inhibitor is a compound selected from the group:
2-phenylethynesulfonamide (Pifithrin-μ);
MKT-077 (I-Ethyl-2-[[3-ethyl-5-(3-methyl-2(3H)-benzothiazolylidene)-4-oxo-2-thiazolidinylidene]methyl]-pyridinium chloride);
methylene blue;
VER155088 (5′-O-[(4-Cyanophenyl)methyl]-8-[[(3,4-dichlorophenyl)methyl]amino]-adenosine);
   or is a combination thereof, or a pharmaceutically acceptable salt thereof.

In certain embodiments, the anti-PESC agent is a combination of each of an HSP70 inhibitor and HSP90 inhibitor, i.e., the combination inhibits both HSP70 and HSP90.

In certain embodiments, the anti-PESC agent is a dual HSP70/HSP90 inhibitor, i.e., inhibits both HSP70 and HSP90. In certain embodiments, the dual inhibitor inhibits each of HSP70 and HSP90 with EC50's preferably within at least 100× of each other, more preferable within 10×, 5× or even 2× of each other. In addition to certain agents described above that can be used as dual HSP70/90 inhibitors,

b. mTOR Inhibitors

In certain embodiments, the anti-PESC agent is an mTor inhibitor.

Non-limiting examples of mTOR inhibitors include rapamycin (sirolimus), everolimus, ridaforolimus, temsirolimus, zotarolimus, rapamycin prodrug AP-23573 (deforolimus), AP-23675, AP-23481, torin-I, torin-2, WYE-354, dactolisib, voxtalisib, omipalisib, apitolisib, vistusertib, gedatolisib, WYE-125132, BGT226, palomid 529, GDC-0349, XL388, CZ415, CC-223, SF1 126, INK128 (MLN0128, Sapanisertib, TAK-288), biolimus-7, biolimus-9 (umirolimus), GSK2126458, OS1027, PP121, Torkinib (PP242), RTB 101, TAM-01, TAM-03, LY294002, CCI-779 (rapamycin 42-ester with 3-hydroxy-2-(hydroxymethyl)-2-methylpropionic acid), AZD8055 ((5-(2,4-bis((S)-3-methylmorpholino)pyrido[2,3-d]pyrimidin-7-yl)-2-methoxyphenyl)methanol); PKI-587 (1-(4-(4-(dimethylamino)piperidine-1-carbonyl)phenyl)-3-(4-(4,6-dimorpholino-1,3,5-triazin-2-yl)phenyl)urea), NVP-BEZ235 (2-methyl-2-{4-[3-methyl-2-oxo-8-(quinolin-3-yl)-2,3-dihydro-1H-imidazo[4-,5-c]quinolin-1-yl]phenyl}propanenitrile), LY294002 ((2-(4-morpholinyl)-8-phenyl-4H-1-benzopyran-4-one), 40-O-(2-hydroxyethyl)-rapamycin; ABT578 (zotarolimus), TAFA-93, 42-O-(methyl-D-glucosylcarbonyl)rapamycin, 42-O-[2-(methyl-D-glucosylcarbonyloxy)ethyl]rapamycin, 31-O-(methyl-D-glucosylcarbonyl)rapamycin, 42-O-(2-hydroxyethyl)-31-O-(methyl-D-glucosylcarbonyl)rapamycin, 42-O-(2-O-methyl-D-fructosylcarbonyl)rapamycin, 42-O-[2-(2-O-methyl-D-fructosylcarbonyloxy)ethyl]rapamycin, 42-O-(2-O-methyl-L-fructosylcarbonyl)rapamycin, 42-O-[2-(2-O-methyl-L-fructosylcarbonyloxy)ethyl]rapamycin, 31-O-(2-O-methyl-D-fructosylcarbonyl)rapamycin, 42-O-(2-hydroxyethyl)-31-O-(2-O-methyl-D-fructosylcarbonyl)rapamycin, 31-O-(2-O-methyl-L-fructosylcarbonyl)rapamycin, 42-O-(2-hydroxyethyl)-31-O-(2-O-methyl-L-fructosylcarbonyl)rapamycin, 42-O-(D-allosylcarbonyl)rapamycin, 42-O-[2-(D-allosylcarbonyloxy)ethyl]rapamycin, 42-O-(L-allosylcarbonyl)rapamycin, 42-O-[2-(L-allosylcarbonyloxy)ethyl]rapamycin, 31-O-(D-allosylcarbonyl)rapamycin, 42-O-(2-hydroxyethyl)-31-O-(D-allosylcarbonyl)rapamycin, 31-O-(L-allosylcarbonyl)rapamycin, 42-O-(2-hydroxyethyl)-31-O-(L-allosylcarbonyl)rapamycin, 42-0-(D-fructosylcarbonyl)rapamycin, 42-O-[2-(D-fructosylcarbonyloxy)ethyl]rapamycin, 42-O-(L-fructosylcarbonyl)rapamycin, 42-O-[2-(L-fructosylcarbonyloxy)ethyl]rapamycin, 31-O-(D-fructosylcarbonyl)rapamycin, 42-O-(2-hydroxyethyl)-31-O-(D-fructosylcarbonyl)rapamycin, 31-O-(L-fructosylcarbonyl)rapamycin, 42-O-(2-hydroxyethyl)-31-O-(L-fructosylcarbonyl)rapamycin, 42-O-(D-fucitolylcarbonyl)rapamycin, 42-O-[2-(D-fucitolylcarbonyloxy)ethyl]rapamycin, 42-O-(L-fucitolylcarbonyl)rapamycin, 42-O-[2-(L-fucitolylcarbonyloxy)ethyl]rapamycin, 31-O-(D-fucitolylcarbonyl)rapamycin, 42-O-(2-hydroxyethyl)-31-O-(D-fucitolylcarbonyl)rapamycin, 31-O-(L-fucitolylcarbonyl)rapamycin, 42-O-(2-hydroxyethyl)-31-O-(L-fucitolylcarbonyl)rapamycin, 42-O-(D-glucalylcarbonyl)rapamycin, 42-O-[2-(D-glucalylcarbonyloxy)ethyl]rapamycin, 42-O-(D-glucosylcarbonyl)rapamycin, 42-O-[2-(D-glucosylcarbonyloxy)ethyl]rapamycin, 42-O-(L-glucosylcarbonyl)rapamycin, 42-O-[2-(L-glucosylcarbonyloxy)ethyl]rapamycin, 31-O-(D-glucalylcarbonyl)rapamycin, 42-O-(2-hydroxyethyl)-31-O-(D-glucalylcarbonyl)rapamycin, 31-0-(D-glucosylcarbonyl)rapamycin, 42-O-(2-hydroxyethyl)-31-O-(D-glucosylcarbonyl)rapamycin, 31-O-(L-glucosylcarbonyl)rapamycin, 42-O-(2-hydroxyethyl)-31-0-(L-glucosylcarbonyl)rapamycin, 42-O-(L-sorbosylcarbonyl)rapamycin, 42-O-(D-sorbosylcarbonyl)rapamycin, 31-O-(L-sorbosylcarbonyl)rapamycin, 31-O-(D-sorbosylcarbonyl)rapamycin, 42-O-[2-(L-sorbosylcarbonyloxy)ethyl]rapamycin, 42-O-[2-(D-sorbosylcarbonyloxy)ethyl]rapamycin, 42-O-(2-hydroxyethyl)-31-O-(D-sorbosylcarbonyl)rapamycin, 42-O-(2-hydroxyethyl)-31-O-(L-sorbosylcarbonyl)rapamycin, 42-O-(D-lactalylcarbonyl)rapamycin, 42-O-[2-(D-lactalylcarbonyloxy)ethyl]rapamycin, 31-O-(D-lactalylcarbonyl)rapamycin, 42-O-(2-hydroxyethyl)-31-O-(D-lactalylcarbonyl)rapamycin, 42-O-(D-sucrosylcarbonyl)rapamycin, 42-O-[2-(D-sucrosylcarbonyloxy)ethyl]rapamycin, 31-0-(D-sucrosylcarbonyl)rapamycin, 42-O-(2-hydroxyethyl)-31-O-(D-sucrosylcarbonyl)rapamycin, 42-O-(D-gentobiosylcarbonyl)rapamycin 42-O-[2-(D-gentobiosylcarbonyloxy)ethyl]rapamycin, 31-O-(D-gentobiosylcarbonyl)rapamycin 42-O-(2-hydroxyethyl)-31-O-(D-gentobiosylcarbonyl)rapamycin 42-O-(D-cellobiosylcarbonyl)rapamycin, 42-O-[2-(D-cellobiosylcarbonyloxy)ethyl]rapamycin, 31-O-(D-cellobiosylcarbonyl)rapamycin, 42-O-(2-hydroxyethyl)-31-O-(D-cellobiosylcarbonyl)rapamycin, 42-O-(D-turanosylcarbonyl)rapamycin, 42-O-[2-(D-turanosylcarbonyloxy)ethyl]rapamycin, 31-O-(D-turanosylcarbonyl)rapamycin, 42-O-(2-hydroxyethyl)-31-O-(D-turanosylcarbonyl)rapamycin, 42-O-(D-palatinosylcarbonyl)rapamycin, 42-O-[2-(D-palatinosylcarbonyloxy)ethyl]rapamycin, 31-O-(D-palatinosylcarbonyl)rapamycin, 42-O-(2-hydroxyethyl)-31-O-(D-palatinosylcarbonyl)rapamycin, 42-O-(D-isomaltosylcarbonyl)rapamycin, 42-O-[2-(D-isomaltosylcarbonyloxy)ethyl]rapamycin, 31-O-(D-isomaltosylcarbonyl)rapamycin, 42-O-(2-hydroxyethyl)-31-O-(D-isomaltosylcarbonyl)rapamycin, 42-O-(D-maltulosylcarbonyl)rapamycin, 42-O-[2-(D-maltulosylcarbonyloxy)ethyl]rapamycin, 42-O-(D-maltosylcarbonyl)rapamycin, 42-O-[2-(D-maltosylcarbonyloxy)ethyl]rapamycin, 31-O-(D-maltulosylcarbonyl)rapamycin, 42-O-(2-hydroxyethyl)-31-O-(D-maltulosylcarbonyl)rapamycin, 31-O-(D-maltosylcarbonyl)rapamycin, 42-O-(2-hydroxyethyl)-31-O-(D-maltosylcarbonyl)rapamycin, 42-O-(D-lactosylcarbonyl)rapamycin, 42-O-[2-(D-lactosylcarbonyloxy)ethyl]rapamycin, 31-O-(methyl-D-lactosylcarbonyl)rapamycin, 42-O-(2-hydroxyethyl)-31-O-(methyl-D-lactosylcarbonyl)rapamycin, 42-O-(D-melibiosylcarbonyl)rapamycin, 31-O-(D-melibiosylcarbonyl)rapamycin, 42-O-(2-hydroxyethyl)-31-O-(D-melibiosylcarbonyl)rapamycin, 42-O-(D-leucrosylcarbonyl)rapamycin, 42-O-[2-(D-leucrosylcarbonyloxy)ethyl]rapamycin, 31-O-(D-leucrosylcarbonyl)rapamycin, 42-0-(2-hydroxyethyl)-31-O-(D-leucrosylcarbonyl)rapamycin, 42-O-(D-raffi nosylcarbonyl)rapamycin, 42-O-[2-(D-raffinosylcarbonyloxy)ethyl]rapamycin, 31-O-(D-raffinosylcarbonyl)rapamycin, 42-O-(2-hydroxyethyl)-31-O-(D-raffinosylcarbonyl)rapamycin, 42-O-(D-isomaltotriosylcarbonyl)rapamycin, 42-O-[2-(D-isomaltosylcarbonyloxy)ethyl]rapamycin, 31-O-(D-isomaltotriosylcarbonyl)rapamycin, 42-O-(2-hydroxyethyl)-31-O-(D-isomaltotriosylcarbonyl)rapamycin, 42-O-(D-cellotetraosylcarbonyl)rapamycin, 42-O-[2-(D-cellotetraosylcarbonyloxy)ethyl]rapamycin, 31-O-(D-cellotetraosylcarbonyl)rapamycin, 42-O-(2-hydroxyethyl)-31-O-(D-cellotetraosylcarbonyl)rapamycin, 42-O-(valiolylcarbonyl)rapamycin, 42-O-[2-(D-valiolylcarbonyloxy)ethyl]rapamycin, 31-O-(valiolylcarbonyl)rapamycin, 42-O-(2-hydroxyethyl)-31-O-(valiolylcarbonyl)rapamycin, 42-O-(valiolonylcarbonyl)rapamycin, 42-O-[2-(D-valiolonylcarbonyloxy)ethyl]rapamycin, 31-O-(valiolonylcarbonyl)rapamycin, 42-O-(2-hydroxyethyl)-31-O-(valiolonylcarbonyl)rapamycin, 42-O-(valienolylcarbonyl)rapamycin 42-0-[2-(D-valienolylcarbonyloxy)ethyl]rapamycin, 31-O-(valienolylcarbonyl)rapamycin, 42-O-(2-hydroxyethyl)-31-O-(valienolylcarbonyl)rapamycin, 42-O-(valienoneylcarbonyl)rapamycin, 42-O-[2-(D-valienoneylcarbonyloxy)ethyl]rapamycin, 31-O-(valienoneylcarbonyl)rapamycin, 42-O-(2-hydroxyethyl)-31-O-(valienoneylcarbonyl)rapamycin, PI-103 (3-[4-(4-morpholinyl)pyrido[3′,2′:4,5]furo[3,2-d]pyrimidin-2-yl]-phenol), KU-0063794 ((5-(2-((2R,6S)-2,6-dimethylmorpholino)-4-morpholinopyrido[2,3-d]pyrimidin-7-yl)-2-methoxyphenyl)methanol), PF-04691502 (2-amino-8-((1r,4r)-4-(2-hydroxyethoxy)cyclohexyl)-6-(6-methoxypyridin-3-yl)-4-methylpyrido[2,3-d]pyrimidin-7(8H)-one), CH132799, RG7422 ((S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one), Palomid 529 (3-(4-methoxybenzyloxy)-8-(1-hydroxyethyl)-2-methoxy-6H-benzo[c]chromen-6-one), PP242 (2-(4-amino-1-isopropyl-1H-pyrazolo[3,4-d]pyrimidin-3-yl)-1H-indol-5-ol), XL765 (N-[4-[[[3-[(3,5-dimethoxyphenyl)amino]-2-quinoxalinyl]amino]sulfon-yl]phenyl]-3-methoxy-4-methyl-benzamide), GSK1059615 ((Z)-5-((4-(pyridin-4-yl)quinolin-6-yl)methylene)thiazolidine-2,4-dione), PKI-587 (1-(4-(4-(dimethylamino)piperidine-1-carbonyl)phenyl)-3-(4-(4,6-dimorpholino-1,3,5-triazin-2-yl)phenyl)urea), WAY-600 (6-(1H-indol-5-yl)-4-morpholino-1-(1-(pyridin-3-ylmethyl)piperidin-4-yl)-1H-pyrazolo[3,4-d]pyrimidine), WYE-687 (methyl 4-(4-morpholino-1-(1-(pyridin-3-ylmethyl)piperidin-4-yl)-1H-pyrazolo[3,4-d]pyrimidin-6-yl)phenylcarbamate), WYE-125132 (N-[4-[1-(1,4-dioxaspiro[4.5]dec-8-yl)-4-(8-oxa-3-azabicyclo[3.2.1]oct-3-yl)-1H-pyrazolo[3,4-d]pyrimidin-6-yl]phenyl]-N′-methyl-urea), and WYE-354; as well as pharmaceutically acceptable salts, hydrates, solvates, or amorphous solid thereof, and combinations thereof.

Additional inhibitors of mTOR are described in the following United States patents and patent applications, all of which are incorporated herein by this reference: U.S. Pat. No. 8,461,157 to Cai et al.; U.S. Pat. No. 8,440,662 to Smith et al.; U.S. Pat. No. 8,436,012 to Ohtsuka et al.; U.S. Pat. No. 8,394,818 to Gray et al.; U.S. Pat. No. 8,362,241 to D'Angelo et al.; U.S. Pat. No. 8,314,111 to Chen et al.; U.S. Pat. No. 8,309,546 to Nakayama et al. (including 6-morpholinopurine derivatives); U.S. Pat. No. 8,268,819 to Jin et al.; U.S. Pat. No. 8,211,669 to Reed et al.; U.S. Pat. No. 8,163,755 Jin et al.; U.S. Pat. No. 8,129,371 Zask et al.; U.S. Pat. No. 8,097,622 to Nakayama et al.; U.S. Pat. No. 8,093,050 to Cho et al.; U.S. Pat. No. 8,008,318 to Beckmann et al.; U.S. Pat. No. 7,943,767 to Chen et al.; U.S. Pat. No. 7,923,555 to Chen et al.; U.S. Pat. No. 7,897,608 to Wilkinson et al.; U.S. Pat. No. 7,700,594 to Chen et al.; U.S. Pat. No. 7,659,274 to Crew et al.; U.S. Pat. No. 7,655,673 to Zhang et al. (39-desmethoxyrapamycin); U.S. Pat. No. 7,648,996 to Beckman et al.; U.S. Pat. No. 7,504,397 to Hummersone et al.; U.S. Pat. No. 7,169,817 to Pan et al.; U.S. Pat. No. 7,160,867 to Abel et al. (carbohydrate derivatives of rapamycin); U.S. Pat. No. 7,091,213 to Metcalf III et al. (“rapalogs”); United States Patent Application Publication No. 2013/0079303 by Andrews et al.; and United States Patent Application Publication No. 2013/0040973 by Vannuchi et al.

The structures of certain mTOR inhibitors are disclosed below:

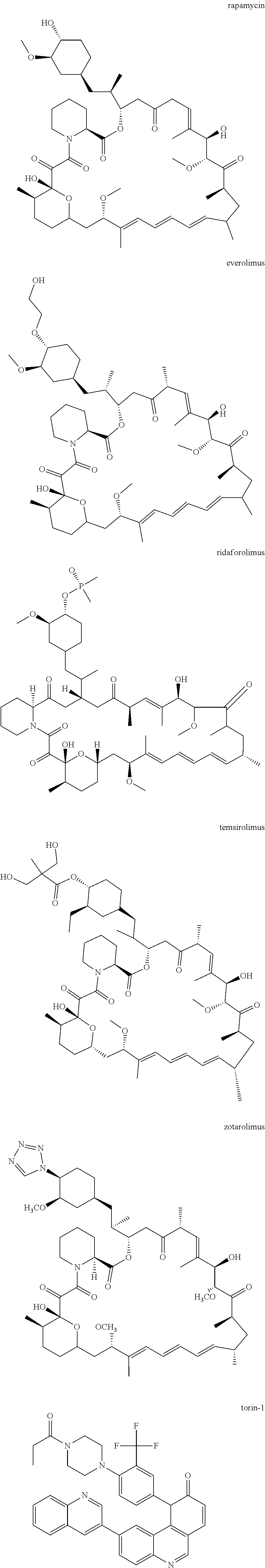

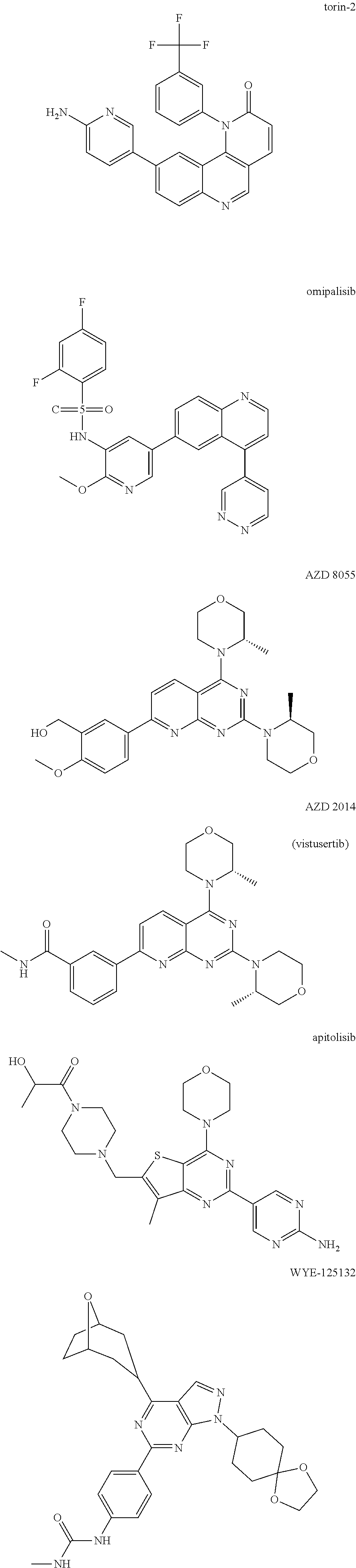

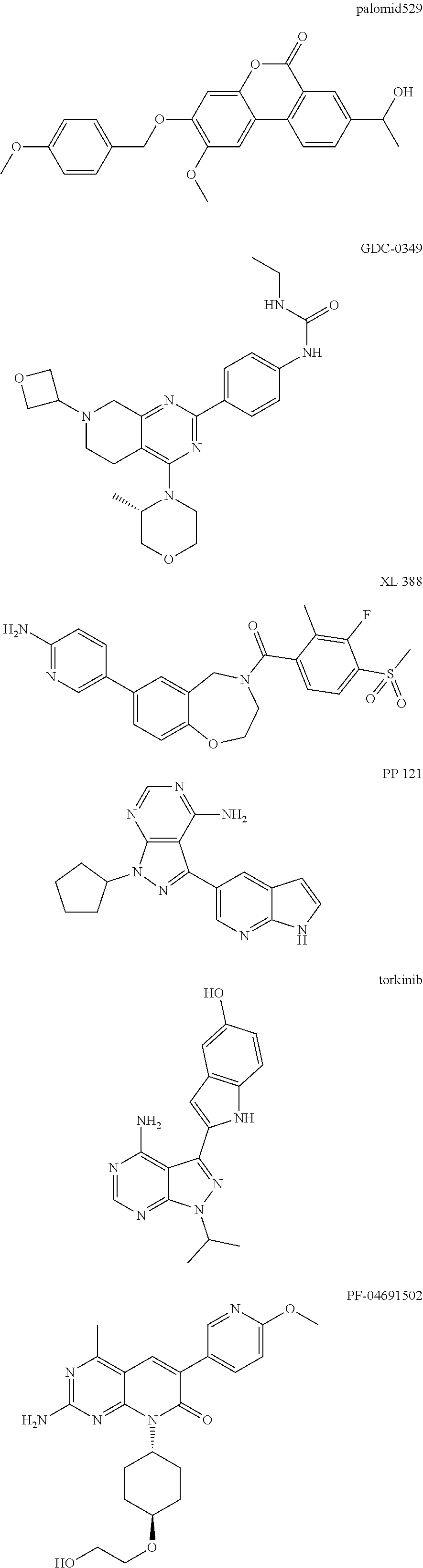 [SMILES: COc1ccc(COc2cc3oc(=O)c4cc(C(C)O)ccc4c3cc2OC)cc1 ; CCNC(=O)Nc1ccc(-c2nc3c(c(N4CCOC[C@@H]4C)n2)CCN(C2COC2)C3)cc1 ; Cc1c(C(=O)N2CCOc3ccc(-c4ccc(N)nc4)cc3C2)ccc(S(C)(=O)=O)c1F ; Nc1ncnc2c1c(-c1cnc3[nH]ccc3c1)nn2C1CCCC1 ; CC(C)n1nc(-c2cc3cc(O)ccc3[nH]2)c2c(N)ncnc21 ; COc1ccc(-c2cc3c(C)nc(N)nc3n([C@H]3CC[C@H](OCCO)CC3)c2=O)cn1]

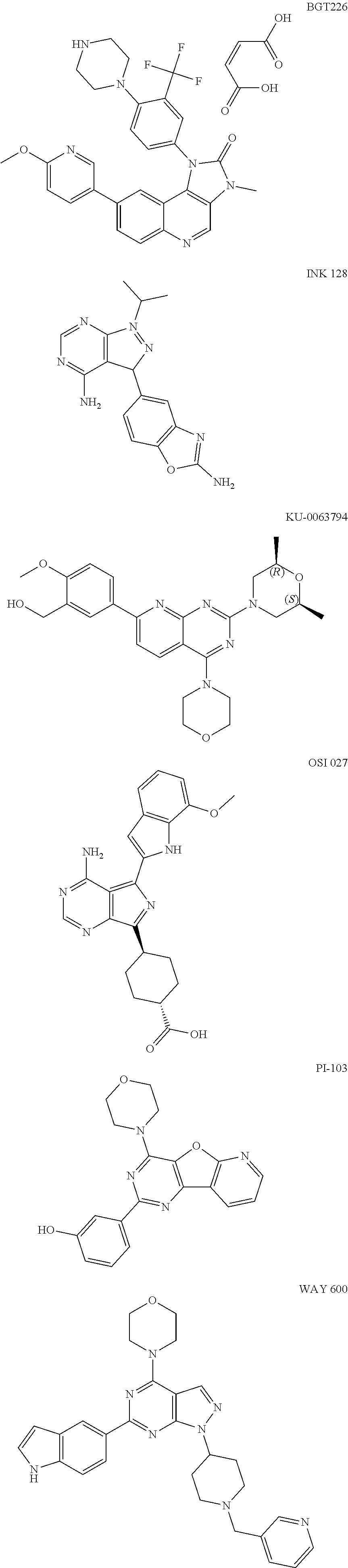

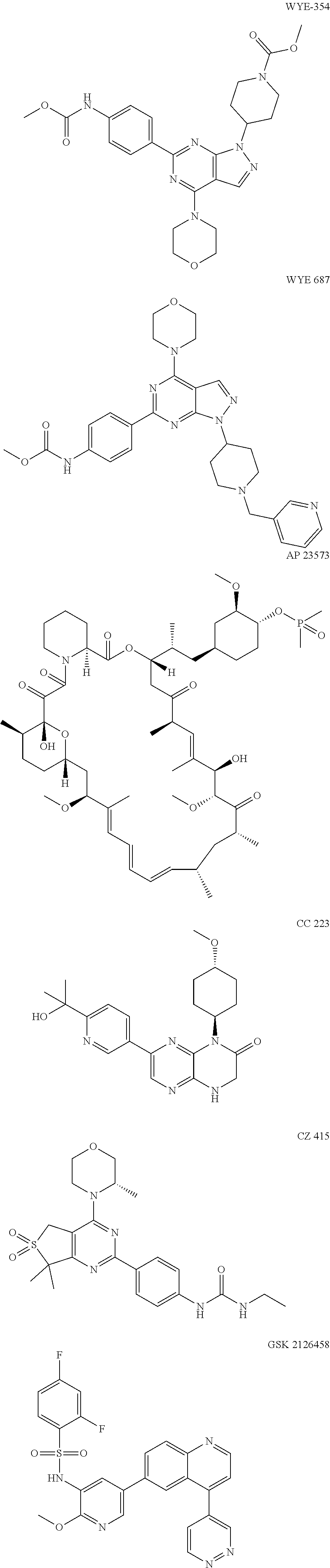

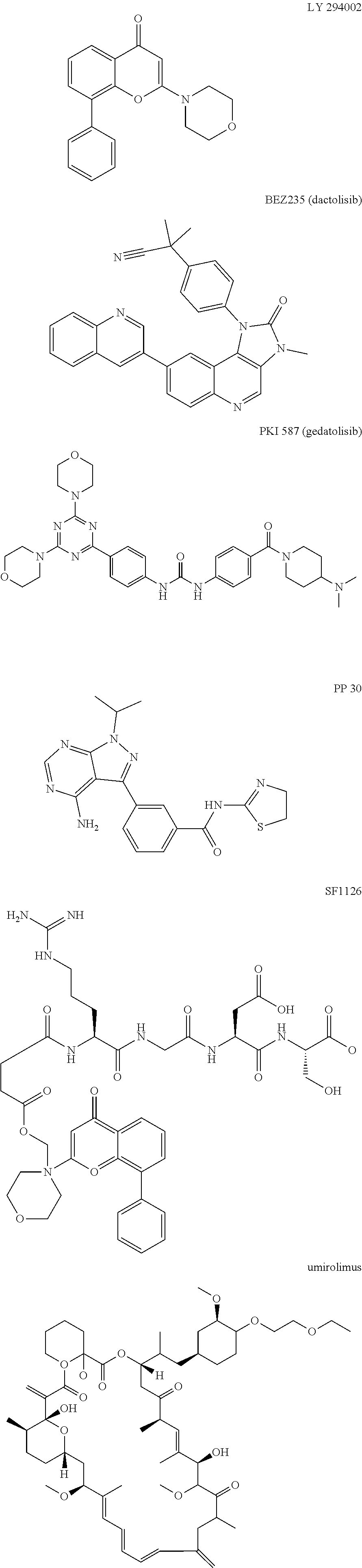

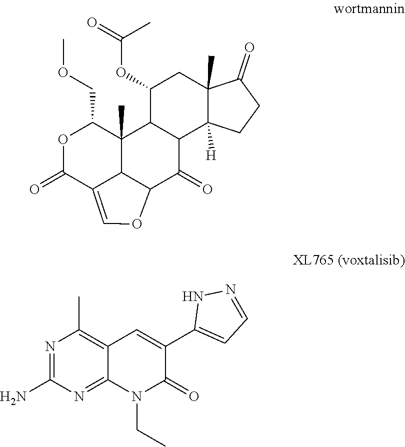 [SMILES: COC[C@H]1OC(=O)C2=COC3C(=O)C4C([C@H](OC(C)=O)C[C@]5(C)C(=O)CC[C@@H]45)[C@]1(C)C23 ; CCn1c(=O)c(-c2ccn[nH]2)cc2c(C)nc(N)nc21]

In some embodiments, mTOR inhibitors also include specific inhibitors of mTOR complex 1, specific inhibitors of mTOR complex 2, and the like. In one embodiment, agents that can be used to inhibit mTOR complex 2 include but are not limited to small molecules, nucleic acids, proteins, and antibodies. Small molecules include but are not limited to pyridinonequinolines, pyrazolopyrimidines, and pyridopyrimidines. In a further embodiment, small molecules that inhibit mTOR complexes 1 and 2 include Torin 1, Torin 2, torkinib (PP242), PP30, KU-0063794, WAY-600, WYE-687, WYE-354, AZD8055, INK128, OS1027, AZD2014, omipalisib, wortmannin, LY294002, PI-103, BGT226, XL765, NVP-BEZ235, RTB IOI (RestorBio), and TAM-01 and TAM-03 (Mount Tam Biotechnologies). In a further embodiment, the inhibitors include but is not limited to antisense oligonucleotide, siRNA, shRNA, and combinations thereof. In a further embodiment, the agent that inhibits mTOR complex 2 would not inhibit mTOR complex 1.

In certain embodiments the mTOR inhibitors also inhibit other mTOR-mediated signaling pathways, an may serve also as inhibitors of, e.g., phosphoinositide 3-kinase (PI3K). Exemplary PI3K/mTOR inhibitors include BTG226, gedatolisib, apitolisib, omipalisib, dactolisib, duvelisib, and idelalisib can be used in lieu of or in addition to mTOR inhibitors. Inhibitors of Akt (Protein Kinase B) such as 8-[4-(1-aminocyclobutyl)phenyl]-9-phenyl-2H-[1,2,4]triazolo[3,4-f][1,6]naphthyridin-3-one; dihydrochloride (MK-2206) also can be used in lieu of or in addition to mTOR inhibitors.

c. RAR Agonists

In another aspect, the agent is an agonist of a retinoic acid receptor (RAR), and preferably a pan-RAR agonist. Known RAR agonists include but are not limited to, TTNPB (4-[(E)-2-(5,6,7,8-Tetrahydro-5,5,8,8-tetramethyl-2-naphthalenyl)-1-propenyl]benzoic acid), tamibarotene, 9-cis-retinoic acid (alitretinoin), all-trans-retinoic acid (tretinoin), AGN193836, Ro 40-6055, CD666, BMS753, isotretinoin, AC261066, AC55649, adapalene, AM580, AM80, BMS961, CD1530, CD2314, CD437, tazarotene, Tazarotenic acid, bexarotene, MDI 301, R667, 9-cis UAB30, LG100268, LGD1069, BMS 270394, BMS 189961, CH 55, LE 135, AM 580, 9CDHRA, Acitretin, AM-580, BMS-453, BMS-493, BMS-753, BMS-961, CD-1530, CD-2314, CD-437, Ch-55, EC 19, EC 23, Etretinate, Fenretinide, Isotretinoin, Palovarotene, and Retinol (vitamin A).

In certain embodiments, the RAR agonist also includes RXR agonist activity.

To further illustrate, the RAR agonist can be

| Name | Specificity | Structure |
|---|---|---|
| Tretinoin | Pan-RAR agonist | 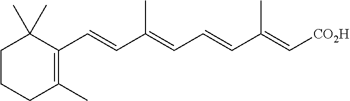 [SMILES: CC1=C(/C=C/C(C)=C/C=C/C(C)=C/C(=O)O)C(C)(C)CCC1] |
| 9-cis RA | Pan-RAR and RXR agonist | 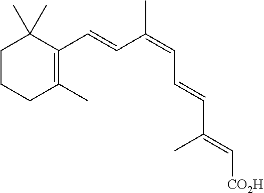 [SMILES: CC1=C(/C=C/C(C)=C\C=C\C(C)=C\C(=O)O)C(C)(C)CCC1] |
| 13-cis-RA | Pan-RAR agonist | 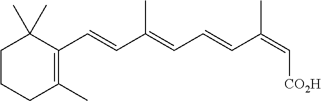 [SMILES: CC1=C(/C=C/C(C)=C/C=C/C(C)=C\C(=O)O)C(C)(C)CCC1] |
| Fenretinide | RAR agonist | 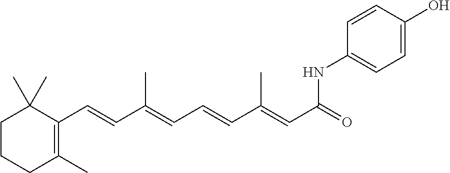 [SMILES: CC1=C(/C=C/C(C)=C/C=C/C(C)=C/C(=O)Nc2ccc(O)cc2)C(C)(C)CCC1] |
| EC 23 | Pan-RAR agonist | 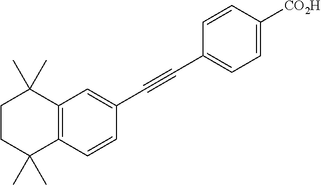 [SMILES: CC1(C)CCC(C)(C)c2cc(C#Cc3ccc(C(=O)O)cc3)ccc21] |
| TTNPB | Pan-RAR agonist | 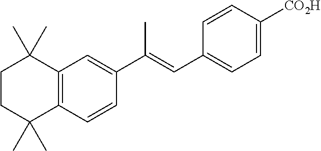 [SMILES: C/C(=C\c1ccc(C(=O)O)cc1)c1ccc2c(c1)C(C)(C)CCC2(C)C] |
| Ch 55 | Pan-RAR agonist | 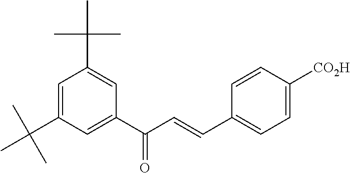 [SMILES: CC(C)(C)c1cc(C(=O)/C=C/c2ccc(C(=O)O)cc2)cc(C(C)(C)C)c1] |
| Tazarotene | RARβ/γ agonist | 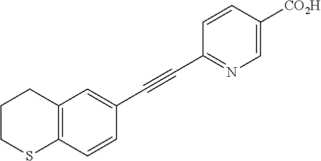 [SMILES: O=C(O)c1ccc(C#Cc2ccc3c(c2)CCCS3)nc1] |
| BMS 753 | RARα agonist | 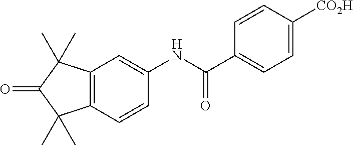 [SMILES: CC1(C)C(=O)C(C)(C)c2cc(NC(=O)c3ccc(C(=O)O)cc3)ccc21] |
| AM80 | RARα agonist | 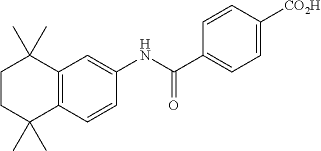 [SMILES: CC1(C)CCC(C)(C)c2cc(NC(=O)c3ccc(C(=O)O)cc3)ccc21] |
| AM580 | RARα agonist | 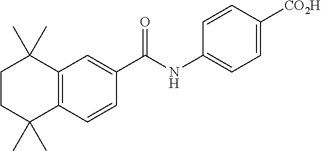 [SMILES: CC1(C)CCC(C)(C)c2cc(C(=O)Nc3ccc(C(=O)O)cc3)ccc21] |
| AC55649 | RARβ2 agonist | 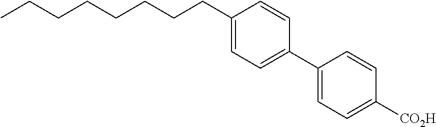 [SMILES: CCCCCCCCc1ccc(-c2ccc(C(=O)O)cc2)cc1] |
| AC261066 | RARβ2 agonist | 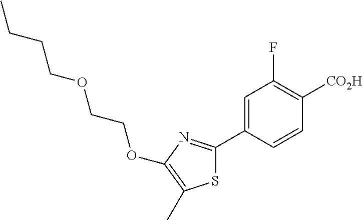 [SMILES: CCCCOCCOc1nc(-c2ccc(C(=O)O)c(F)c2)sc1C] |
| Adapalene | RARβ and γ agonist | 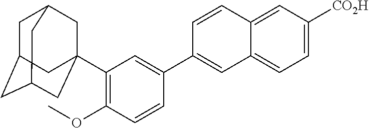 [SMILES: COc1ccc(-c2ccc3cc(C(=O)O)ccc3c2)cc1C12CC3CC(CC(C3)C1)C2] |
| CD437 | RARγ agonist | 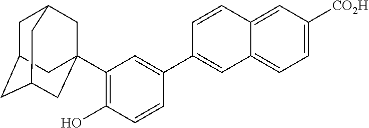 [SMILES: O=C(O)c1ccc2cc(-c3ccc(O)c(C45CC6CC(CC(C6)C4)C5)c3)ccc2c1] |
| CD1530 | RARγ agonist | 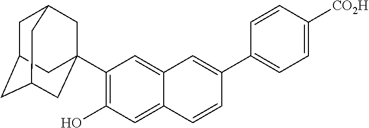 [SMILES: O=C(O)c1ccc(-c2ccc3cc(O)c(C45CC6CC(CC(C6)C4)C5)cc3c2)cc1] |
| CD2665 | RARγ agonist | 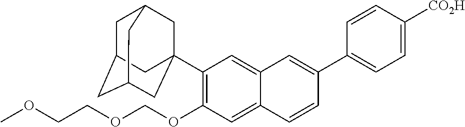 [SMILES: COCCOCOc1cc2ccc(-c3ccc(C(=O)O)cc3)cc2cc1C12CC3CC(CC(C3)C1)C2] |
| MM11253 | RAR agonist | 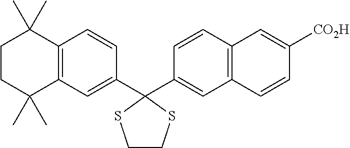 [SMILES: CC1(C)CCC(C)(C)c2cc(C3(c4ccc5cc(C(=O)O)ccc5c4)SCCS3)ccc21] |
| LE135 | RARβ agonist | 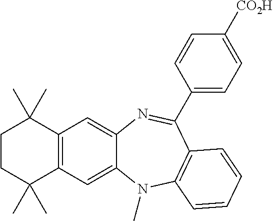 [SMILES: CN1c2cc3c(cc2N=C(c2ccc(C(=O)O)cc2)c2ccccc21)C(C)(C)CCC3(C)C] |

d. Proteasome Inhibitors

In still another aspect, the agent is a proteasome inhibitor, preferably an immunoproteasome inhibitor.

The proteasome inhibitor may be any proteasome inhibitor known in the art. In particular, it is one of the proteasome inhibitors described in more detail in the following paragraphs.

Exemplary proteasome inhibitors include bortezomib, carfilzomib, ixazomib, oprozomib, marizomib, CEP-18770, disulfiram, epigallocatechin-3-gallate, epoxomicin, lactacystin, MG132, MLN9708, ONX 0912, PR-924, PR-957, KZR-504, LMP7-IN-1, salinosporamide A, epoxomycine, eponemycine, aclacinomycine A (aclarubicine), celastrol, withaferin A, Gliotoxin, epipolythiodioxo-piperazines, green tea polyphenolic catechins (−)-epigallocatechin-3-gallate, Disulfuram, acridine derivatives, tetra-acridine derivatives with betulinic acid, as 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides, TMC-95A, TMC-95A endocyclic oxindole-phenyl clamp (BIA-1a) derivatives, TMC-95A endocyclic biphenyl-ether clamp (BIA-2a) derivatives, lactacystine, Omuralide, Homobelactosin C, Salinosporamide A, NEOSH-101, CEP-18770, IPS1001, IPS1007, MLN2238, MLN9708, ONX 0914, AA-102, 26 S PI, AVR-147, 4E12, N-carbobenzoxy-L-leucinyl-L-leucinyl-1-leucinal and its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)2, pyrazyl-2,5-bis-CONH(CHPhe)CONH(CHisobutyl)-B(OH)2, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S, 4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)2 and its enantiomer PS-293, 8-quinolyl-sulfonyl-CONH—(CH-napthyl)-CONH(—CH-isobutyl)-B(OH)2, NH2(CH-Napthyl)-CONH—(CH-isobutyl)-B(OH)2, morpholino-CONH—(CH-napthyl)-CONH—(CH-phenylalanine)-B(OH)2, CH3-NH—(CH-napthyl-CONH—(CH-isobutyl)-B(OH)2, 2-quinole-CONH—(CH-homo-phenylalanin)-CONH—(CH-isobutyl)-B(OH)2, Phenyalanine-CH2-CH2-CONH—(CH-phenylalanine)-CONH—(CH-isobutyl)-B(OH)2, “PS-383” (pyridyl-CONH—(CHpF-phenylalanine)-CONH—(CH-isobutyl)-B(OH)2, (PEG)19-25-Leu-Leu-Nle-H, (PEG)19-25-Arg-Val-Arg-H, H-Nle-Leu-Leu-(PEG)19-25-Leu-Leu-Nle-H, H-Arg-Val-Arg-(PEG)19-25-Arg-Val-Arg-H ZLLL-vs), ZLLVS, YLVS, MG-262, ALLnL, ALLnM, LLnV, DFLB Ada-(Ahx)3-(Leu)3-vs, YU101 (Ac-hFLFL-ex), MLN519 and S-2209.

To further illustrate, in certain embodiments suitable proteasome inhibitors for use in combinations described herein include (a) peptide boronates, such as bortezomib (also known as Velcade™ and PS341), delanzomib (also known as CEP-18770), ixazomib (also known as MLN9708) or ixazomib citrate; (b) peptide aldehydes, such as MG132 (Z-Leu-Leu-Leu-H), MG115 (Z-Leu-Leu-Nva-H), IPSI 001, fellutamide B, ALLN (Ac-Leu-Leu-N₁e-H, also referred to as calpain inhibitor I), and leupeptin (Ac-Leu-Leu-Arg-al); (c) peptide vinyl sulfones, (d) epoxyketones, such as epoxomicin, oprozomib (also referred to as PR-047 or ONX 0912), PR-957 (also known as ONX 0914), and carfilzomib (also referred to as PR-171); and (e) p-lactones, such as lactacystin, omuralide, salinosporamide A (also known as NPI-0052 and marizomib), salinosporamide B, belactosines, cinnabaramides, polyphenols, TMC-95, and PS-519.

In certain preferred embodiments, the proteasome inhibitor is a boronic acid class inhibitor, i.e., such as a peptide borinic acid, such as a dipeptide or tripeptide boronic acid.

In certain embodiments, the proteasome inhibitor is bortezomib, also known as VELCADE and PS341. In a preferred embodiment, the proteasome inhibitor is [(1R)-3-methyl-1-[[(2S)-3-phenyl-2-(pyrazine-2-carbonylamino)propanoyl]amino]butyl]boronic acid. In a preferred embodiment, the proteasome inhibitor is the compound of Formula:

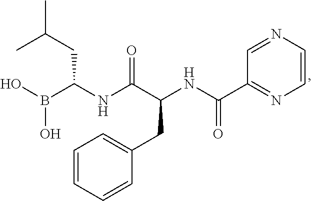 [SMILES: CC(C)C[C@H](NC(=O)[C@H](Cc1ccccc1)NC(=O)c1cnccn1)B(O)O]

or a pharmaceutically acceptable salt, solvate, hydrate, cocrystal, or prodrug thereof.

In certain embodiments, the proteasome inhibitor is delanzomib, also known as CEP-18770 or [(1R)-1-[[(2S,3R)-3-hydroxy-2-[(6-phenylpyridine-2-carbonyl)amino]butanoyl]amino]-3-methylbutyl]boronic acid. In a preferred embodiment, the proteasome inhibitor is the compound of Formula:

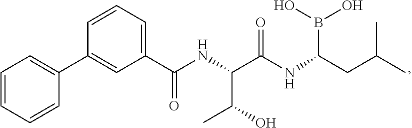 [SMILES: CC(C)C[C@H](NC(=O)[C@@H](NC(=O)c1cccc(-c2ccccc2)c1)[C@@H](C)O)B(O)O]

or a pharmaceutically acceptable salt, solvate, hydrate, cocrystal, or prodrug thereof.

In certain embodiments, the proteasome inhibitor is ixazomib, also known as MLN-9708 or ixazomib citrate or 4-(carboxymethyl)-24(R)-1-(2-(2,5-dichlorobenzamido)acetamido)-3-methylbutyl)-6-oxo-1,3,2-dioxaborinane-4-carboxylic acid, or a pharmaceutically acceptable salt, solvate, hydrate, cocrystal, or prodrug thereof.

In certain embodiment, the proteasome inhibitor is 1,3,2-dioxaborolane-4,4-diacetic acid, 2-[(1R)-1-[[2-[(2,5-dichlorobenzoyl)amino] acetyl] amino]-3-methylbutyl]-5-oxo-, or a pharmaceutically acceptable salt, solvate, hydrate, cocrystal, or prodrug thereof.

In a preferred embodiment, the proteasome inhibitor is 2,2′-{2-[(1R)-1-{[N-(2,5-dichlorobenzoyl)glycyl]amino}-3-methylbutyl]-5-oxo-1,3,2-dioxaborolane-4,4-diyl}diacetic acid, or a pharmaceutically acceptable salt, solvate, hydrate, cocrystal, or prodrug thereof.

In a preferred embodiment, the proteasome inhibitor is the compound of Formula:

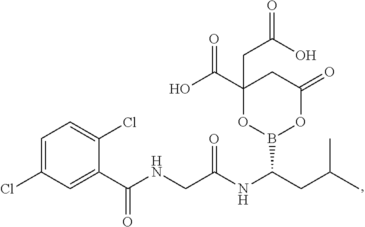 [SMILES: CC(C)C[C@H](NC(=O)CNC(=O)c1cc(Cl)ccc1Cl)B1OC(=O)CC(CC(=O)O)(C(=O)O)O1]

or a pharmaceutically acceptable salt, solvate, hydrate, cocrystal, or prodrug thereof.

In certain embodiments, the proteasome inhibitor is 1B-{(1R)-1-[2-(2,5-dichlorobenzamido)acetamido]-3-methylbutyl}boronic acid, or a pharmaceutically acceptable salt, solvate, hydrate, cocrystal, or prodrug thereof. In a preferred embodiment, the proteasome inhibitor is the compound of Formula:

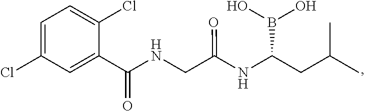 [SMILES: CC(C)C[C@H](NC(=O)CNC(=O)c1cc(Cl)ccc1Cl)B(O)O]

or a pharmaceutically acceptable salt, solvate, hydrate, cocrystal, or prodrug thereof.

In certain embodiments, the proteasome inhibitor is marizomib, also known as NPI-0052 and Salinosporamide A or (4R,5S)-4-(2-chloroethyl)-1-((1S)-cyclohex-2-enyl(hydroxy)methyl)-5-methyl-6-oxa-2-azabicyclo[3.2.0]heptane-3,7-dione, or a pharmaceutically acceptable salt, solvate, hydrate, cocrystal, or prodrug thereof. In a preferred embodiment, the proteasome inhibitor is the compound of Formula:

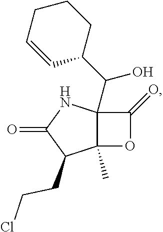 [SMILES: C[C@@]12OC(=O)C1(C(O)[C@@H]1C=CCCC1)NC(=O)[C@@H]2CCCl]

or a pharmaceutically acceptable salt, solvate, hydrate, cocrystal, or prodrug thereof.

In certain preferred embodiments, the proteasome inhibitor is an epoxyketone class inhibitor, i.e., such as an peptide epoxyketone, such as a tetrapeptide epoxyketone or tripeptide epoxyketone, and may be an analog of epoxomicin.

In one embodiment, the protoeasome inhibitor is carfilzomib, also known as PX-171-007, or (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4 phenylbutanamido)-4-methylpentanamide, or a pharmaceutically acceptable salt, solvate, hydrate, cocrystal, or prodrug thereof. In certain embodiments, the proteasome inhibitor is the compound of Formula:

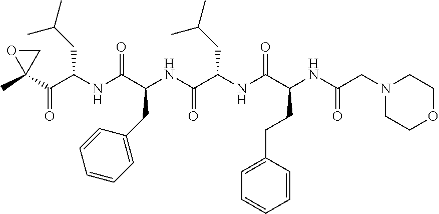 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

In certain embodiments, the proteasome inhibitor is oprozimib, also known as PR-047 or ONX 0912, or N-[(2S)-3-methoxy-1-[[(2S)-3-methoxy-1-[[(2S)-1-[(2R)-2-methyloxiran-2-yl]-1-oxo-3-phenylpropan-2-yl]amino]-1-oxopropan-2-yl]amino]-1-oxopropan-2-yl]-2-methyl-1,3-thiazole-5-carboxamide, or a pharmaceutically acceptable salt, solvate, hydrate, cocrystal, or prodrug thereof. In certain embodiments, the proteasome inhibitor is the compound of Formula:

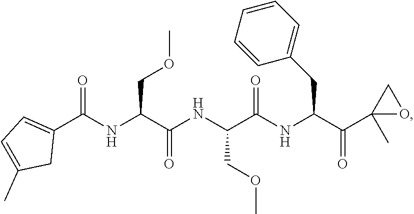 [SMILES: COC[C@H](NC(=O)C1=CC=C(C)C1)C(=O)N[C@@H](COC)C(=O)N[C@@H](Cc1ccccc1)C(=O)C1(C)CO1]

In certain preferred embodiments, the epoxyketone is an immunoproteasome inhibitor, i.e., is inhibitor of β5i/LMP7, and even more preferably is a selective inhibitor of β5i/LMP7.

In certain embodiments, the proteasome inhibitor is oprozimib, also known as PR-957 or ONX 0914, or (2S)-3-(4-methoxyphenyl)-N-[(2S)-1-(2-methyloxiran-2-yl)-1-oxo-3-phenylpropan-2-yl]-2-[[(2S)-2-[(2-morpholin-4-ylacetyl)amino]propanoyl]amino]propanamide, or is a pharmaceutically acceptable salt, solvate, hydrate, cocrystal, or prodrug thereof. In certain embodiments, the proteasome inhibitor is the compound of Formula:

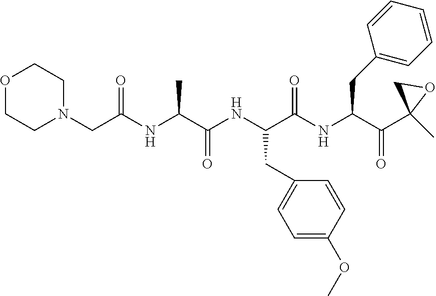

Other exemplary immunoproteasome inhibitors include:

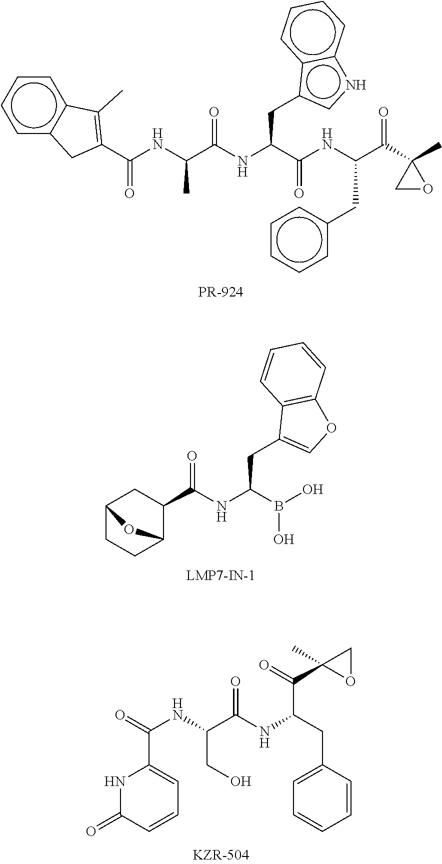

d. BCR-ABL Kinase Inhibitor

In another aspect, the agent is a tyrosine kinase inhibitor, preferably an ABL1 Kinase Inhibitor, and more preferably is a BCR-ABL Kinase inhibitor. Examples of BCR-ABL tyrosine kinase inhibitors include imatinib, dasatinib, nilotinib, bosutinib, ponatinib, bafetinib, saracatinib, tozasertib and rebastinib.

| Drug | Structure |
|---|---|
| Imatinib (STI571) | 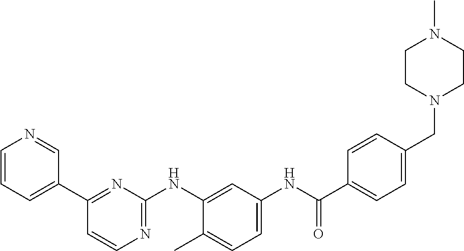 [SMILES: Cc1ccc(NC(=O)c2ccc(CN3CCN(C)CC3)cc2)cc1Nc1nccc(-c2cccnc2)n1] |
| Nilotinib (AMN107) | 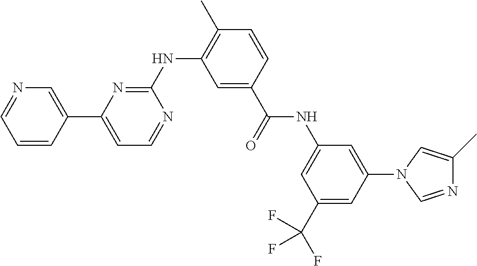 [SMILES: Cc1cn(-c2cc(NC(=O)c3ccc(C)c(Nc4nccc(-c5cccnc5)n4)c3)cc(C(F)(F)F)c2)cn1] |
| Dasatinib (BMS-345825) | 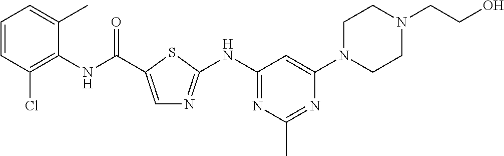 [SMILES: Cc1nc(Nc2ncc(C(=O)Nc3c(C)cccc3Cl)s2)cc(N2CCN(CCO)CC2)n1] |
| Bosutinib (SKI-606) | 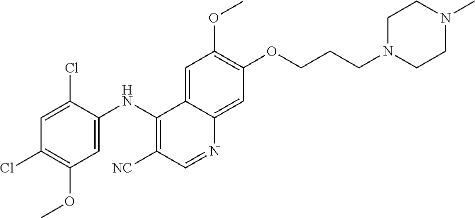 [SMILES: COc1cc(Nc2c(C#N)cnc3cc(OCCCN4CCN(C)CC4)c(OC)cc23)c(Cl)cc1Cl] |
| Ponatinib (AP-24534) | 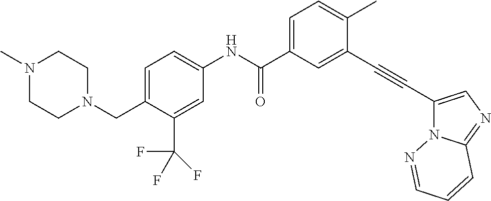 [SMILES: Cc1ccc(C(=O)Nc2ccc(CN3CCN(C)CC3)c(C(F)(F)F)c2)cc1C#Cc1cnc2cccnn12] |
| Bafetinib (INNO-406) | 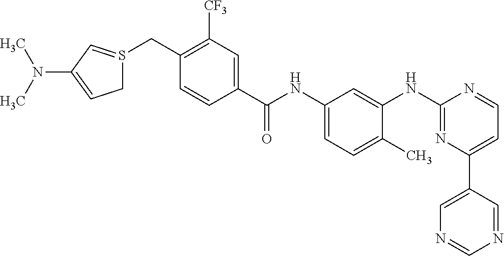 [SMILES: Cc1ccc(NC(=O)c2ccc(CS3=CC(N(C)C)=CC3)c(C(F)(F)F)c2)cc1Nc1nccc(-c2cncnc2)n1] |

e. EGFR Inhibitor

In certain embodiments, the anti-PESC agent is a receptor tyrosine kinase inhibitor, and is preferably an EGFR inhibitor, a HER2 inhibitor or a dual EGFR/HER2 inhibitor.

Exemplary EGFR inhibitors/antagonists include, inter alia, small-molecule EGFR inhibitors/antagonists, such as gefitinib, erlotinib, lapatinib, afatinib (also referred to as BIBW2992), neratinib, ABT-414, dacomitinib (also referred to as PF-00299804), AV-412, PD 153035, vandetanib, PKI-166, pelitinib (also referred to as EKB-569), canertinib (also referred to as CI-1033), icotinib, poziotinib (also referred to as NOV120101), BMS-690514, CUDC-101, AP26113, XL647, AZD9291, CO-1686 (rotsiletinib), WZ4002, PF 00299804, BDTX-189, mavelertinib, JBJ-04-125-02, AG-490, tucatinib, genistein, pyrotinib, sapitinib, mobocertinib, AZ-5104, mubritinib, zorifertinib, rociletinib, lazertinib, lifirafenib, butein, PD168393, PD153035, daphnetin, tarloxtinib, and icotinib.

WZ8040 is a novel mutant-selective irreversible EGFRT790M inhibitor, does not inhibit ERBB2 phosphorylation (T7981).

In certain embodiments, the anti-PESC agent is an EGFR tyrosine kinase inhibitor (EGFR-TKI). Exemplary EGFR-TKI include afatinib, erlotinib, gefitinib, icotinib, neratinib, dacomitinib and osimertinib.

In certain embodiments, the EGFR tyrosine kinase inhibitor is erlotinib.

f. Rock (Rho-Kinase) Inhibitor

In certain embodiments, the anti-PESC agent is a Rock (Rho-kinase) Inhibitor. Exemplary ROCK inhibitors include GSK269962A, Chroman, Fasudil, Hydroxyfasudil, Rapasudil, Narciclasine, Afuresertib, Thiazovivn, Y-33075, AT13148, Belumsudil, Verosudil, CRT0066854, GSK180736A, BDP5290, SAR407899, GSK-25, ROCK-IN-1, (R)-(+)-trans-N-(4-Pyridyl)-4-(1-aminoethyl)-cyclohexanecarboxamide) dihydrochloride monohydrate (Y-27632, SigmaAldrich), 5-(1,4-diazepan-1-ylsulfonyl)isoquinoline (fasudil or HA1077, Cayman Chemical), SAR407899, CPMD101, (1S,)-(+)-2-methyl-1-[(4methyl-5-isoquinolinyl)sulfonyl]-hexahydro-1H-1,4-diazepine dihydrochloride (HI 152, Tocris Bioscience), and N-(6-fluoro-1H-indazol-5-yl)-2-methyl-6-oxo-4-(4-(trifluoromethyl)phenyl)-1,4,5,6-tetrahydropyridine-3-carboxamide (GSK429286A, Stemgent), ripasudil, netarsudil, RKI-1447, Y-27632, H-1152P, INS-115644, Y-39983, SB772077BS, LX7101, AR-12286, H-1337 and Y-21632.

In certain embodiments, the ROCK inhibitor is fasudil, Y-27632, H-1152P, INS-115644, Y-39983, SB772077BS, LX7101, AR-12286, H-1337, ripasudil, netarsudi or verosudil.

g. MELK Inhibitors

In certain embodiments, the anti-PESC agent is an inhibitor of the maternal embryonic leucine zipper kinase (MELK). Exemplary MELK inhibitors include OTSSP167, WJ-7-25-1, HTH-01-091, MRT199665, NVS-MELK8a and MELK-T1.

In certain embodiments, the MELK inhibitor is OTSSP167.

h. SRC Kinase Inhibitors

In certain embodiments, the anti-PESC agent is an inhibitor of Src kinases. Exemplary Src Kinase inhibitors include PP1, PP2, SU6656, eCF506, A-419259, UM-164, A-419259, KX1-004, KX2-391, CGP 77675, WH-4-023, MCB-613, A-419259, Saracatinib, Dasatinib, Bosutinib, MNS, Src Kinase Inhibitor 1.

In certain embodiments, the Src Kinase inhibitor is PP1, PP2, KX2-391, Saracatinib, Dasatinib or Bosutinib.

i. Inhibiting Expression of Anti-PESC Targets

In addition to using small molecule inhibitors of the anti-PESC targets above, another aspect of the disclosure relates to the use of the nucleic acid therapeutics to reduce or inhibit the expression of the target of the anti-PESC drug (“anti-PESC Gene Target”), such as to inhibit expression of HSP90, HSP70, mTOR, RAR, proteaseome or immunoprotease subunits, BCR-ABL kinase or the a combination thereof. Eemplary nucleci acid therapeutics can include antisense therapy or RNA intereference therapy (such as small interfering RNA (siRNA), micro RNA (miRNA) or short-hairpin RNA (shRNA)), a sequence-directed ribozyme or gene inactivating CRISPR RNA (crRNA).

As used herein, antisense therapy refers to administration or in situ generation of oligonucleotide molecules or their derivatives which specifically hybridize (e.g., bind) under cellular conditions with the cellular mRNA and/or genomic DNA, thereby inhibiting transcription and/or translation of that gene. The binding may be by conventional base pair complementarity, or, for example, in the case of binding to DNA duplexes, through specific interactions in the major groove of the double helix. In general, antisense therapy refers to the range of techniques generally employed in the art, and includes any therapy which relies on specific binding to oligonucleotide sequences.

An antisense construct of the present disclosure can be delivered, for example, as an expression plasmid which, when transcribed in the cell, produces RNA which is complementary to at least a unique portion of the cellular mRNA. Alternatively, the antisense construct is an oligonucleotide probe which is generated ex vivo and which, when introduced into the cell, causes inhibition of expression by hybridizing with the mRNA and/or genomic sequences of a subject nucleic acid. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphorothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al., BioTechniques 6:958-976 (1988); and Stein et al., Cancer Res. 48:2659-2668 (1988). With respect to antisense DNA, oligodeoxyribonucleotides derived from the translation initiation site, e.g., between the −10 and +10 regions of the nucleotide sequence of interest, are preferred.

Antisense approaches involve the design of oligonucleotides (either DNA or RNA) that are complementary to mRNA. The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well (Wagner, Nature 372:333 (1994)). Therefore, oligonucleotides complementary to either the 5′ or 3′ untranslated, non-coding regions of a gene could be used in an antisense approach to inhibit translation of endogenous mRNA. Oligonucleotides complementary to the 5′ untranslated region of the mRNA should include the complement of the AUG start codon. Antisense oligonucleotides complementary to mRNA coding regions are typically less efficient inhibitors of translation but could also be used in accordance with the disclosure. Whether designed to hybridize to the 5, 3, or coding region of subject mRNA, antisense nucleic acids should be at least six nucleotides in length, and are preferably less that about 100 and more preferably less than about 50, 25, 17 or 10 nucleotides in length.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al., Proc. Natl. Acad. Sci. U.S.A. 93:14670 (1996) and in Eglom et al., Nature 365:566 (1993). One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an—anomeric oligonucleotide. An—anomeric oligonucleotide forms specific double-stranded hybrids with complementary RNA in which, contrary to the usual—units, the strands run parallel to each other (Gautier et al., Nucl. Acids Res. 15:6625-6641 (1987)). The oligonucleotide is a 2-O-methylribonucleotide (Inoue et al., Nucl. Acids Res. 15:6131-12148 (1987)), or a chimeric RNA-DNA analogue (Inoue et al., FEBS Lett. 215:327-330 (1987)).

The antisense molecules can be delivered to cells which express the target nucleic acid in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

In another aspect of the disclosure, ribozyme molecules designed to catalytically cleave target mRNA transcripts corresponding to one or more anti-PESC Gene Target can be used to prevent translation of target mRNA and expression of a target protein by the IBD stem cell or its progeny (See, e.g., PCT International Publication WO90/11364; Sarver et al., Science 247:1222-1225 (1990) and U.S. Pat. No. 5,093,246). While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5-UG-3. The construction and production of hammerhead ribozymes is well known in the art and is described more fully in Haseloff and Gerlach, 1988, Nature, 334:585-591. Preferably the ribozyme is engineered so that the cleavage recognition site is located near the 5′ end of the target mRNA; i.e., to increase efficiency and minimize the intracellular accumulation of non-functional mRNA transcripts.

The ribozymes of the present disclosure also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., Science, 224:574-578 (1984); Zaug and Cech, Science, 231:470-475 (1986); Zaug, et al., Nature, 324:429-433 (1986); published International patent application No. WO88/04300; Been and Cech, Cell, 47:207-216 (1986)). The Cech-type ribozymes have an eight base pair active site which hybridizes to a target RNA sequence whereafter cleavage of the target RNA takes place. The disclosure encompasses those Cech-type ribozymes which target eight base-pair active site sequences that are present in a target anti-PESC Gene Target.

As in the antisense approach, the ribozymes can be composed of modified oligonucleotides (e.g., for improved stability, targeting, etc.) and should be delivered to cells which express the target anti-PESC Gene Target in vivo. A preferred method of delivery involves using a DNA construct “encoding” the ribozyme under the control of a strong constitutive pol III or pol II promoter, so that transfected cells will produce sufficient quantities of the ribozyme to destroy endogenous messages and inhibit translation. Because ribozymes, unlike antisense molecules, are catalytic, a lower intracellular concentration is required for efficiency.

Antisense RNA, DNA, RNA Interference constructs and ribozyme molecules of the disclosure may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

In other embodiments, the nucleic acid is a “decoy” nucleic acid which corresponds to a transcriptional regulatory sequence and binds to a transcription factor that is involved in upregulated expression of one or more genes in an IBD Stem Cell population. The decoy nucleic acid therefore competes with natural binding target for the binding of the transcription factor and acts an antagonist to reduce the expression of those genes under the transcriptional control of the targeted transcription factor.

Increased efficiency can also be gained through other techniques, such as in which delivery of the therapeutic nucleic acid is improved by use of chemical carriers-cationic polymers or lipids—or via a physical approach—gene gun delivery or electroporation. See Tranchant et al. (2004) “Physicochemical optimisation of plasmid delivery by cationic lipids” J. Gene Med., 6 (Suppl. 1):S24-S35; and Niidome et al. (2002) “Gene therapy progress and prospects: nonviral vectors” Gene Ther., 9:1647-1652. Electroporation is especially regarded as an interesting technique for nonviral gene delivery. Somiari, et al. (2000) “Theory and in vivo application of electroporative gene delivery” Mol. Ther. 2:178-187; and Jaroszeski et al. (1999) “In vivo gene delivery by electroporation” Adv. Drug Delivery Rev., 35:131-137. With electroporation, pulsed electrical currents are applied to a local tissue area to enhance cell permeability, resulting in gene transfer across the membrane. Research has shown that in vivo gene delivery can be at least 10-100 times more efficient with electroporation than without. See, for example, Aihara et al. (1998) “Gene transfer into muscle by electroporation in vivo” Nat. Biotechnol. 16:867-870; Mir, et al. (1999) “High-efficiency gene transfer into skeletal muscle mediated by electric pulses” PNAS 96:4262-4267; Rizzuto, et al. (1999) “Efficient and regulated erythropoietin production by naked DNA injection and muscle electroporation” PNAS 96: 6417-6422; and Mathiesen (1999) “Electropermeabilization of skeletal muscle enhances gene transfer in vivo” Gene Ther., 6:508-514.

The therapeutic nucleic acids of the present disclosure can be delivered by a wide range of gene delivery system commonly used for gene therapy including viral, non-viral, or physical. See, for example, Rosenberg et al., Science, 242:1575-1578, 1988, and Wolff et al., Proc. Natl. Acad. Sci. USA 86:9011-9014 (1989). Discussion of methods and compositions for use in gene therapy include Eck et al., in Goodman & Gilman's The Pharmacological Basis of Therapeutics, Ninth Edition, Hardman et al., eds., McGraw-Hill, New York, (1996), Chapter 5, pp. 77-101; Wilson, Clin. Exp. Immunol. 107 (Suppl. 1):31-32, 1997; Wivel et al., Hematology/Oncology Clinics of North America, Gene Therapy, S. L. Eck, ed., 12(3):483-501, 1998; Romano et al., Stem Cells, 18:19-39, 2000, and the references cited therein. U.S. Pat. No. 6,080,728 also provides a discussion of a wide variety of gene delivery methods and compositions. The routes of delivery include, for example, systemic administration and administration in situ.

### IV. Normal GI Stem Cell Promoters

The inventors have also observed that certain of the drug agents they screened were able to selectively promote the proliferation and regenerative capabilities of normal GI stem cells, relative to Crohn's disease stem cells, i.e., are ESO Regenerative Agents.

a. BACE Inhibitors

In certain embodiments, the ESO Regenerative Agent is a β-secretase (BACE) inhibitor, and more preferably a selective BACE1 inhibitor.

A number of BACE1 inhibitors are known in the art, including small molecules and inhibitory antibodies. BACE1 inhibitors include LY2886721 and LY2811376 (Lilly); MBI-1, MBI-3, MBI-5, and MK-8931 (Merck); E2609 (Eisai); RG7129 (Roche); TAK-070 (Takeda); CTS-21166 (CoMentis); AZ3971, AZ4800, AZD-3289, AZD-3293 and AZ4217 (AstraZeneca); HPP854 (High Point Pharmaceuticals); Ginsenoside Rg1 (CID 441923); Hispidin (CID310013); TDC (CID 5811533); Monacolin K (CID 53232); SCH 1359113; Spirocyclic inhibitors (e.g., as described in Hunt et al., J Med Chem. 2013 Apr. 25; 56(8):3379-403, such as compound (R)-50); fluorine-substituted 1,3-oxazines (e.g., as described in Hilpert et al., J Med Chem. 2013 May 23; 56(10):3980-95, such as the CF3 substituted oxazine 89). Inhibitory antibodies include bispecific antibodies with one arm targeting BACE and the other recognizing transferrin receptor to boost brain penetrance (see, e.g., Yu et al., Sci Transl Med. 2011 May 25; 3(84):84ra44; Atwal et al., Sci Transl Med. 2011 May 25; 3(84):84ra43, and U.S. Pat. No. 8,772,457) and camelid antibodies that bind and inhibit BACE1 encoded by virus (see e.g., U.S. Pat. No. 8,568,717 and US20110091446).

Other exemplary BACE1 inhibitors include AM-6494; AMG-8718; Anisomycin; Atabecestat; Aurapten; C000000956; CL82198; Corynoline; Donepezil; EBI-2511; Elenbecestat; Felbinac; Ginsenoside Re; L 651580; L 655240; L 8412; Laciniatoside V; Lanabecestat (i.e., such as free base or camsylate); Lanabecestat (also known as AZD3293 and LY3314814); LDN-57444; Loganin; Methylguanidine hydrochloride; NB-360 (particularly the free base form); PF-05297909; PF-06663195; PF-06751979 (particularly the free base form); PH-002; R05508887 (particularly the free base form); Sinensetin; Taxifolin; Tolfenamic acid; Trientine-2HCI (also known as Triethylenetetramine, abbreviated TETA and trien); Umibecestat (particularly the free base form or HCl salt); Verubecestat (particularly the free base and TFA forms).

These and other BACE1 inhibitors useful in the present methods are described in the following US Pre-Grant Publications: 20140286963; 20140275165; 20140235626; 20140228356; 20140228277; 20140186357; 20140179690; 20140112867; 20140057927; 20140051691; 20140011802; 20130289050; 20130217705; 20130210839; 20130108645; 20130105386; 20120258961; 20120245157; 20120245155; 20120245154; 20120238557; 20120237526; 20120232064; 20120214186; 20120202828; 20120202804; 20120190672; 20120172355; 20120171120; 20120148599; 20120094984; 20120093916; 20120064099; 20120015961; 20110288083; 20110237576; 20110207723; 20110158947; 20110152341; 20110152253; 20110091446; 20110071124; 20110033463; 20100317850; 20100285597; 20100273671; 20100221760; 20100144790; 20100132060; 20100093999; 20100075957; 20100063134; 20090258925; 20090209755; 20090176836; 20090162878; 20090136977; 20090081731; 20090060987; 20090042993; 20080124379; 20070224656; 20070185042; 20060216292; 20060182736; 20060178328; 20060052327; 20050196398; 20050048641; 20040248231; 20040220132; 20040162255; 20040132680; 20040063161; 20030194745; 20020159991; and 20020157122, and U.S. Pat. Nos. 8,772,457; 8,703,785; 8,568,717; 8,415,319; 8,288,354; 8,198,269; 8,183,219; 8,058,251; 7,829,694; 7,816,378; 7,618,948; 7,273,743; and 6,713,276.

b. FAK Inhibitors

Focal adhesion kinase (FAK), also known as cytoplasmic protein-tyrosine kinase (PTK2), is a cytosolic protein tyrosine kinase concentrated in the focal adhesions that form among cells attaching to extracellular matrix constituents.

In certain embodiments, the ESO Regenerative Agent is an inhibitor of focal adhesion kinase (FAK), i.e., is a FAK Inhibitor. Exemplary FAK inhibitors include PF-562271, PF-00562271, PND-1186, GSK2256098, PF-431396, PF-4618433, TAE226, CEP-37440, PF-03814735, PF-573228, BI-4464, NVP-TAE 226, PND-1186 and Defactinib. In certain embodiments, the FAK inhibitor is a Dual FAK/PYK2 inhibitor such as PF-431396. In other embodiments, the FAK inhibitor is a selective FAK inhibitor, such as FAK Inhibitor 14, PF-573228 or Y-11.

The structures of exemplary inhibitors of FAK are provided in the table below.

| Inhibitor Name | |
|---|---|
| PF-562271 | 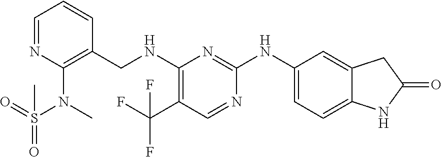 [SMILES: CN(c1ncccc1CNc1nc(Nc2ccc3c(c2)CC(=O)N3)ncc1C(F)(F)F)S(C)(=O)=O] |
| PF-573228 | 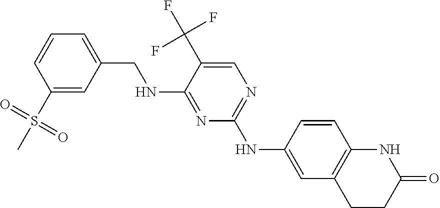 [SMILES: CS(=O)(=O)c1cccc(CNc2nc(Nc3ccc4c(c3)CCC(=O)N4)ncc2C(F)(F)F)c1] |
| TAE226 (NVP- TAE226) | 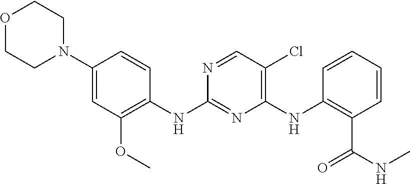 [SMILES: CNC(=O)c1ccccc1Nc1nc(Nc2ccc(N3CCOCC3)cc2OC)ncc1Cl] |
| PF-03814735 | 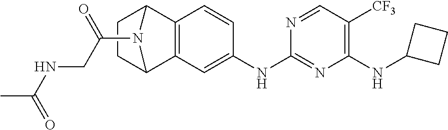 [SMILES: CC(=O)NCC(=O)N1C2CCC1c1cc(Nc3ncc(C(F)(F)F)c(NC4CCC4)n3)ccc12] |
| PF-562271 HCl | 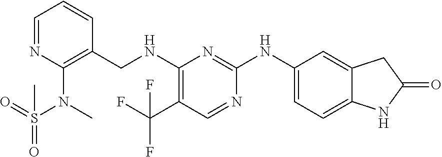 [SMILES: CN(c1ncccc1CNc1nc(Nc2ccc3c(c2)CC(=O)N3)ncc1C(F)(F)F)S(C)(=O)=O] |
| GSK2256098 | 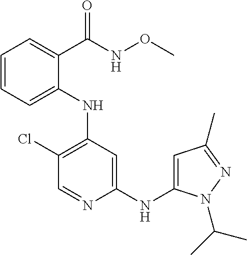 [SMILES: CONC(=O)c1ccccc1Nc1cc(Nc2cc(C)nn2C(C)C)ncc1Cl] |
| PF-431386 | 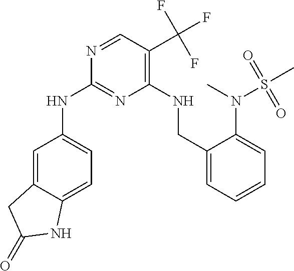 [SMILES: CN(c1ccccc1CNc1nc(Nc2ccc3c(c2)CC(=O)N3)ncc1C(F)(F)F)S(C)(=O)=O] |
| PND-1186 (VS- 4718) | 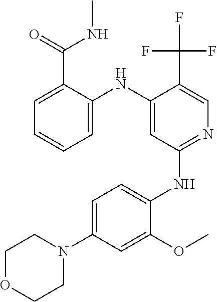 [SMILES: CNC(=O)c1ccccc1Nc1cc(Nc2ccc(N3CCOCC3)cc2OC)ncc1C(F)(F)F] |
| Detactinib (VS- 6063, PF- 04554878) | 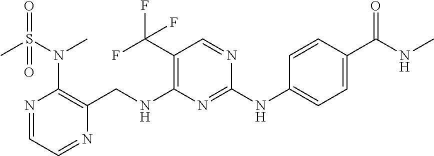 |
| Solanesol (Nonaisoprenol) | 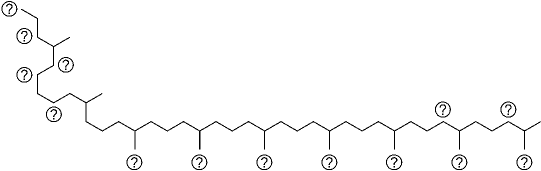 [SMILES: CCCC(C)CCCCCC(C)CCCC(C)CCCC(C)CCCC(C)CCCC(C)CCCC(C)CCCC(C)CCCC(C)C] |

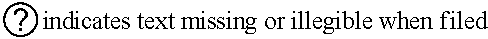

c. VEGFR Inhibitors

In certain embodiments, the ESO Regenerative Agent is a VEGF receptor pathway inhibitor, preferably a VEGF receptor tyrosine kinase inhibitor. Exemplary VEGF receptor pathway inhibitors include vatalanib succinate (or other compounds disclosed in EP 296122), bevacizumab (AVASTIN®), axitinib (INLYTA®), brivanib alaninate (BMS-582664, (S)—((R)-1-(4-(4-Fluoro-2-methyl-1H-indol-5-yloxy)-5-methylpyrrolo[2,1-f-][1,2,4]triazin-6-yloxy)propan-2-yl)2-aminopropanoate), sorafenib (NEXAVAR®), pazopanib (VOTRIENT®), sunitinib malate (SUTENT®), cediranib (AZD2171, CAS 288383-20-1), vargatef (BIBF1120, CAS 928326-83-4), Foretinib (GSK1363089), telatinib (BAY57-9352, CAS 332012-40-5), apatinib (YN968D1, CAS 811803-05-1), imatinib (GLEEVEC®), ponatinib (AP24534, CAS 943319-70-8), tivozanib (AV951, CAS 475108-18-0), regorafenib (BAY73-4506, CAS 755037-03-7), vatalanib dihydrochloride (PTK787, CAS 212141-51-0), brivanib (BMS-540215, CAS 649735-46-6), vandetanib (CAPRELSA® or AZD6474), motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470), dovitinib dilactic acid (TK1258, CAS 852433-84-2), linfanib (ABT869, CAS 796967-16-3), cabozantinib (XL184, CAS 849217-68-1), lestaurtinib (CAS 111358-88-4), N-[5-[[[5-(1,1-dimethylethyl)-2-oxazolyl]methyl]thio]-2-thiazolyl]-4-piperidinecarboxamide (BMS38703, CAS 345627-80-7), (3R,4R)-4-amino-1-((4-((3-methoxyphenyl)amino)pyrrolo[2,1-f][1,2,4]triazin-5-yl)methyl)piperidin-3-ol (BMS690514), N-(3,4-Dichloro-2-fluorophenyl)-6-methoxy-7-[[(3α,5β,6aα-)-octahydro-2-methylcyclopenta[c]pyrrol-5-yl]methoxy]-4-quinazolinamine (XL647, CAS 781613-23-8), 4-methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0), aflibercept (EYLEA®), and endostatin (ENDOSTAR®).

In some embodiment, the VEGFR inhibitor is an inhibitor of one or more of VEGFR-2, PDGFR□KIT or Raf kinase C, 1-methyl-5-((2-(5-(trifluoromethyl)-1H-imidazol-2-yl)pyridin-4-yl)oxy)-N-(4-(trifluoromethyl)phenyl)-1H-benzo[d]imidazol-2-amine (Compound A37) or a compound disclosed in PCT Publication No. WO 2007/030377.

d. AKT Inhibitors

In certain embodiments, the ESO Regenerative Agent is an AKT Inhibitor such as GDC0068 (also known as GDC-0068, ipatasertib and RG7440), MK-2206, perifosine (also known as KRX-0401), GSK690693, AT7867, triciribine, CCT128930, A-674563, PHT-427, Akti-1/2, afuresertib (also known as GSK2110183), AT13148, GSK2141795, BAY1125976, uprosertib (aka GSK2141795), Akt Inhibitor VIII (1,3-dihydro-1-[1-[[4-(6-phenyl-1H-imidazo[4,5-g]quinoxalin-7-yl)phenyl]methyl]-4-piperidinyl]-2H-benzimidazol-2-one), Akt Inhibitor X (2-chloro-N,N-diethyl-10H-phenoxazine-10-butanamine, monohydrochloride), MK-2206 (8-(4-(1-aminocyclobutyl)phenyl)-9-phenyl-[1,2,4]triazolo[3,4-f][-1,6]naphthyridin-3(2H)-one), uprosertib (N—((S)-1-amino-3-(3,4-difluorophenyl)propan-2-yl)-5-chloro-4-(4-chloro-1-methyl-1H-pyrazol-5-yl)furan-2-carboxamide), ipatasertib ((S)-2-(4-chlorophenyl)-1-(4-((5R,7R)-7-hydroxy-5-methyl-6,7-dihydro-5H-cyclopenta[d]pyrimidin-4-yl)piperazin-1-yl)-3-(isopropylamino)propan-1-one)-, AZD 5363 (4-Piperidinecarboxamide, 4-amino-N-[(1S)-1-(4-chlorophenyl)-3-hydroxypropyl]-1-(7H-pyrrolo[2,3-d]pyrimidin-4-yl)), perifosine, GSK690693, GDC-0068, tricirbine, CCT128930, A-674563, PF-04691502, AT7867, miltefosine, PHT-427, honokiol, triciribine phosphate, and KP372-1A (10H-indeno[2,1-e]tetrazolo[1,5-b][1,2,4]triazin-10-one), Akt Inhibitor IX (CAS 98510-80-6).

Additional Akt inhibitors include: ATP-competitive inhibitors, e.g., isoquinoline-5-sulfonamides (e.g., H-8, H-89, NL-71-101), azepane derivatives (e.g., (−)-balanol derivatives), aminofurazans (e.g., GSK690693), heterocyclic rings (e.g., 7-azaindole, 6-phenylpurine derivatives, pyrrolo[2,3-d]pyrimidine derivatives, CCT128930, 3-aminopyrrolidine, anilinotriazole derivatives, spiroindoline derivatives, AZD5363, A-674563, A-443654), phenylpyrazole derivatives (e.g., AT7867, AT13148), thiophenecarboxamide derivatives (e.g., Afuresertib (GSK2110183), 2-pyrimidyl-5-amidothiophene derivative (DC120), uprosertib (GSK2141795); Allosteric inhibitors, e.g., 2,3-diphenylquinoxaline analogues (e.g., 2,3-diphenylquinoxaline derivatives, triazolo[3,4-f][1,6]naphthyridin-3(2H)-one derivative (MK-2206)), alkylphospholipids (e.g., Edelfosine (1-O-octadecyl-2-O-methyl-rac-glycero-3-phosphocholine, ET-18-OCH3) ilmofosine (BM 41.440), miltefosine (hexadecylphosphocholine, HePC), perifosine (D-21266), erucylphosphocholine (ErPC), erufosine (ErPC3, erucylphosphohomocholine), indole-3-carbinol analogues (e.g., indole-3-carbinol, 3-chloroacetylindole, diindolylmethane, diethyl 6-methoxy-5,7-dihydroindolo [2,3-b]carbazole-2,10-dicarboxylate (SR13668), OSU-A9), Sulfonamide derivatives (e.g., PH-316, PHT-427), thiourea derivatives (e.g., PIT-1, PIT-2, DM-PIT-1, N-[(1-methyl-1H-pyrazol-4-yl)carbonyl]-N′-(3-bromophenyl)-thiourea), purine derivatives (e.g., Triciribine (TCN, NSC 154020), triciribine mono-phosphate active analogue (TCN-P),4-amino-pyrido[2,3-d]pyrimidine derivative API-1, 3-phenyl-3H-imidazo[4,5-b]pyridine derivatives, ARQ 092), BAY 1125976, 3-methyl-xanthine, quinoline-4-carboxamide, 2-[4-(cyclohexa-1,3-dien-1-yl)-1H-pyrazol-3-yl]phenol, 3-oxo-tirucallic acid, 3.alpha.- and 3.beta.-acetoxy-tirucallic acids, acetoxy-tirucallic acid; and irreversible inhibitors, e.g., natural products, antibiotics, Lactoquinomycin, Frenolicin B, kalafungin, medermycin, Boc-Phe-vinyl ketone, 4-hydroxynonenal (4-HNE), 1,6-naphthyridinone derivatives, and imidazo-1,2-pyridine derivatives.

### V. Local Delivery

The disclosure provides for use of these drug agents, systemically of by localized delivery to the GI tract of patients, in order to more effectively treat IBD and other inflammatory diseases/conditions of the gut, as well as forms of metaplasia, neoplasia and cancers of the gastrointestinal tract. In certain embodiments, one or both of the inhibitor and promoter are formulated, together or separately, for local delivery to GI tract, and (preferably) are released in the terminal ileum.

Merely to illustrate an embodiment, the present disclosure provides a colon targeted bioadhesive modified release formulation, comprising a promoter and/or inhibitor as described above, or a pharmaceutically acceptable salt. For instance, the formulation can comprise a bioadhesive coating that is disposed over all or a portion of the surface of a core containing one or more of the subject drug agents, which core may optionally be coated with a rate-controlling membrane system, thus yielding a monolithic system that releases the agent in a regulated manner. Representative synthetic polymers for use in bioadhesive coatings include polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Other polymers suitable for use in the disclosure include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, and polyvinylphenol. Representative bioerodible polymers for use in bioadhesive coatings include polylactides, polyglycolides and copolymers thereof, poly(ethylene terephthalate), poly(butyric acid), poly(valeric acid), poly(lactide-co-caprolactone), poly[lactide-co-glycolide], polyanhydrides (e.g., poly(adipic anhydride)), polyorthoesters, blends and copolymers thereof.

Polyanhydrides are particularly suitable for use in bioadhesive delivery systems because, as hydrolysis proceeds, causing surface erosion, more and more carboxylic groups are exposed to the external surface. However, polylactides erode more slowly by bulk erosion, which is advantageous in applications where it is desirable to retain the bioadhesive coating for longer durations. In designing bioadhesive polymeric systems based on polylactides, polymers that have high concentrations of carboxylic acid are preferred. The high concentrations of carboxylic acids can be attained by using low molecular weight polymers (MW of 2000 or less), because low molecular weight polymers contain a high concentration of carboxylic acids at the end groups.

When the bioadhesive polymeric coating is a synthetic polymer coating, the synthetic polymer is typically selected from polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, polystyrene, polymers of acrylic and methacrylic esters, polylactides, poly(butyric acid), poly(valeric acid), poly(lactide-co-glycolide), polyanhydrides, polyorthoesters, poly(fumaric acid), poly(maleic acid), and blends and copolymers of thereof. In an exemplary embodiment, the synthetic polymer is poly(fumaric-co-sebacic) anhydride.

Another group of polymers suitable for use as bioadhesive polymeric coatings are polymers having a hydrophobic backbone with at least one hydrophobic group pendant from the backbone. Suitable hydrophobic groups are groups that are generally non-polar. Examples of such hydrophobic groups include alkyl, alkenyl and alkynyl groups. Preferably, the hydrophobic groups are selected to not interfere and instead to enhance the bioadhesiveness of the polymers.

A further group of polymers suitable for use as bioadhesive polymeric coatings are polymers having a hydrophobic backbone with at least one hydrophilic group pendant from the backbone. Suitable hydrophilic groups include groups that are capable of hydrogen bonding or electrostatically bonding to another functional group. Example of such hydrophilic groups include negatively charged groups such as carboxylic acids, sulfonic acids and phosphonic acids, positively charged groups such as (protonated) amines and neutral, polar groups such as amides and imines. Preferably, the hydrophilic groups are selected to not interfere and instead to enhance the bioadhesiveness of the polymers. The hydrophilic groups can be either directly attached to a hydrophobic polymer backbone or attached through a spacer group. Typically, a spacer group is an alkylene group, particularly a C1-C8 alkyl group such as a C2-C6 alkyl group. Preferred compounds containing one or more hydrophilic groups include amino acids (e.g., phenyalanine, tyrosine and derivatives thereof) and amine-containing carbohydrates (sugars) such as glucosamine.

a. Formulation Approaches for Targeted Drug Delivery to the Terminal Ileum

Colon targeted drug delivery systems are designed to selectively release a drug in response to the colonic environment without premature drug release in the upper GI tract.

pH-Dependent Drug Delivery Systems. The colon exhibits a relatively higher pH than the upper GI tract, and this can be used as a targeting strategy for colonic drug delivery. Accordingly, a colon-targeted drug delivery system is designed by using pH-dependent polymers such as cellulose acetate phthalates (CAP), hydroxypropyl methyl-cellulose phthalate (HPMCP) 50 and 55, copolymers of methacrylic acid and methyl methacrylate (e.g., Eudragit® S 100, Eudragit® L, Eudragit® FS, and Eudragit® P4135 F). Particularly, Eudragit polymers are the most widely used synthetic copolymers for colonic drug delivery that offer mucoadhesiveness and pH-dependent drug release. The ideal polymer should be able to withstand the low pH of the stomach and the proximal part of the small intestine but be dissolved by the pH of the terminal ileum and the colon. As a result, drug delivery systems coated with pH-dependent polymers having a dissolution threshold of pH 6.0-7.0 are expected to delay the drug dissolution and prevent premature drug release in the upper GI tract before reaching colonic sites. However, this pH-dependent system has demonstrated significant variability in drug release and failure in vivo due to the vast inter- and intra-subject variability in critical parameters including pH, fluids volumes, GI transit times, and motility. Furthermore, pH ranges of GI tract can be significantly altered by diet, disease state, water intake, and microbial metabolism. For example, patients with ulcerative colitis exhibit more acidic colonic pH compared to healthy humans, leading to incomplete drug release from enteric coated systems at the target site. Thus, the dynamic pH change by many internal and external factors may attenuate the efficiency of pH-dependent drug release systems, and those skilled in the art can appropriately compensate for pH at the intended site of release under the disease conditions.

For instance, to address pH-dependent delivery there have been combinations of pH-dependent systems with other delivery systems including time-dependent systems and enzyme-triggered systems. For example, Eudragit S were blended with high-amylose maize starch for the integration of pH-dependent system and colonic microbial degradation systems. Those skilled in the art will know how to adopted dual coating approach by using the alkaline aqueous solution of Eudragit S with buffering agents for inner layer and the organic solution of Eudragit S for outer layer, accelerating the drug dissolution at pH>7. The in vivo performance of such dual coated system has been evaluated in humans, demonstrating more consistent disintegration of dual coated tablets mainly in the lower intestinal tract. In other embodiments, microspheres combining time- and pH-dependent systems for colonic delivery can be used. By using a combination of Eudragit S and ethyl cellulose, for example, one can achieve greater colonic drug delivery while preventing premature drug release in the upper intestine. Eudracol is another example of a multi-unit technology providing targeted drug delivery to the colon, with delayed and uniform drug release, which can be adapted to deliver the anti-PESC drugs of the current disclosure. This system is based on coating the pellet with Eudragit RL/RS and Eudragit FS 30D, providing colon-specific drug release in a pH- and time-dependent manner. Overall, integrated systems of the different release-triggering mechanisms can be more helpful to overcome the pathophysiological variability compared to pH-dependent system alone.

In addition, nano-/micro-particles also hold great potential for specifically targeting inflamed colonic tissues and enhance drug uptake. Accordingly, various formulations that have combined a pH-dependent system with particle size reduction have been developed for colon-targeted drug delivery.

Polymer-Based Nano-/Micro-Particles. Many studies have demonstrated that pH-dependent polymeric nanoparticles are effective as colonic drug delivery systems. For instance, the subject anti-PESC agents can be delivered using pH-sensitive hydrolyzed polyacrylamide-grafted-xanthan gum (PAAm-g-XG) for colon-targeted delivery. Furthermore, the blended mixture of two different pH-sensitive polymers can be used to control the drug release rate. Drug-loaded nanoparticles can be generated, to illustrate, by using the combination of Eudragit L100 and Eudragit S100. In other embodiments, nanoparticles can be prepared with Eudragit FS30D and Eudragit RS100, using an oil-in-water emulsion solvent evaporation method. Eudragit FS30D is a pH-dependent polymer that dissolves in an environment above pH 7.0, while Eudragit RS100 is a time-dependent, controlled-release polymer having low permeability. Combining these two polymers effectively minimized premature drug release in the upper GI tract and achieved sustained-drug release throughout the colon. Furthermore, in colitis mice models, these pH-/time-dependent nanoparticles delivered drugs more efficiently to the inflamed colonic sites.

Lipid-Based Formulations. Liposomes are an efficient drug delivery system composed of double-layered phospholipids. Liposomes are biodegradable, biocompatible, and amenable to the incorporation of both hydrophilic and lipophilic drugs. The surface of liposomes can be coated with pH-dependent polymers to avoid the destabilization of liposomes in acidic conditions and also with ligands to improve the site-specificity. For example, colon-targeted liposomal formulations for anti-PESC agents can be created by coating the surface of anionic liposomes with glycol chitosan and pH-dependent Eudragit S100. These liposomes can have high stability at acidic and neutral pHs with minimal drug leakage.

Solid lipid nanoparticles are also a superior system in terms of drug protection, entrapment efficiency, and increasing the amount of drug released at specific sites. The lipid matrix of solid lipid nanoparticles degrades at a slow rate and allows for extended drug release.

Self-microemulsifying drug delivery system (SMEDDS) have immense potential for enhancing the oral bioavailability of various hydrophobic drugs, which can be useful in the design of colon-targeted drug delivery systems in the present disclosure. For example, folate-modified SMEDDS (FSMEDDS) containing anti-PESC drugs, which are then filled into soft capsules coated with Eudragit S 100, can be generated. These FSMEDDS formulation can efficiently bind to folate receptors on colon cells.

Tablets and Capsules. Colon targeted drug delivery can be achieved with film coated tablets or capsules. To illustrate, Eudragit L100-coated tablets can be used for the colonic delivery of anti-PESC agents. These tablets can exhibit sustained drug release at pH≥6 but no drug release during 2-hr incubation in acidic conditions. In vivo studies in monkeys also supported the sustained release in the intestine for the topical treatment of IBD. In addition, the drug release profiles can be manipulated by using a combination of copolymers with varying the ratios. This combination system may be superior to tablets coated with a single polymer for colon-targeted drug delivery.

Therefore, there have been continuous efforts to improve the targeting effectiveness via the multi-unit formulations based on the integration of the different mechanism-based systems with pH-dependent coating. For example, one skilled in the art can prepare an anti-PESC agent-loaded multi-unit tablet by coating with different combinations of pH-dependent polymers (Eudragit S and Eudragit L) and time-dependent polymer (Eudragit RS). Drug release from the optimized tablet can be minimal in gastric and intestinal fluids while extensive drug release is observed in colonic fluid. In another embodiment, an effective colonic delivery system is based on the combination of time-dependent and pH-dependent approaches, which is prepared by successive coating of a tablet core with low-viscosity HPMC and Eudragit L.

Zein is a potential carrier for controlled-release solid dispersion systems delivering poorly water soluble drugs to the colon since it is resistant to low pH environments. Recently, a single-layer film coating of tablets using biopolymer Zein in combination with Kollicoat MAE 100P showed high potential to prevent the drug release in the upper GI tract for the delayed drug release in the colon. The ratio of the coating components and the thickness of the coating layer play an important role in the performance of coated tablets for colonic drug delivery.

In recent years, new coating technology has been actively pursued to improve the targeting effectiveness of pH-dependent delivery systems. For example, ColoPulse technology is an innovative pH responsive coating technology, which incorporates super-disintegrant in the coating matrix to accelerate the disintegration at the target site. The incorporation of a super-disintegrant in a non-percolating mode leads to a more reliable and pulsatile drug release. Previous studies demonstrated that ColoPulse tablets enabled the site-specific delivery of the active substance to the ileo-colonic region of Crohn's patients as well as healthy subjects. Furthermore, food and time of food intake do not affect the targeting effectiveness of ColoPulse delivery systems.

Preparation of capsule shell with built-in gastroresistance is another approach for site-specific drug delivery. These gastroresistant capsule shells may have some advantages including large production using a typical high-speed capsule filler, encapsulation of diverse drugs, and potentially reducing research and development costs. To illustrate one method for producing enteric capsule shells without any additional coating steps, different enteric capsule shells can be used to target various region of GI tract, such as by using cellulose derivatives (HPMC AS-LF and HP-55) along with acrylic/methacrylic acid derivatives (Eudragit L100 and Eudragit S100).

Enzyme-Sensitive Drug Delivery Systems—Polysaccharide-Based Systems. Microbiota-activated delivery systems have shown promise in colon-targeted drug delivery due to the abrupt increase of microbiota and the associated enzymatic activities in the lower GI tract. These systems are dependent on the specific enzyme activity of the colonic bacteria and the polymers degradable by colonic microorganisms. Particularly, polysaccharides such as pectin, guar gum, inulin, and chitosan have been used in colon-targeted drug delivery systems, because they can retain their integrity in the upper GI tract but are metabolized by colonic microflora to release the entrapped drug. Recently, new polysaccharides including arabinoxylans and agave fructans are also being explored for colonic drug delivery systems. Furthermore, structural modifications or derivatives of polysaccharides can improve drug release behavior, stability, and site specificity. Mucoadhesiveness of polysaccharides can be advantageous for drug uptake via the prolonged contact between the mucosal surface and drug delivery carriers. Polysaccharide-based delivery systems also have some additional advantages including availability at large scale, relatively low cost, low toxicity and immunogenicity, high biocompatibility, and biodegradability. Consequently, the polysaccharide-based, microbiota-triggered system is promising strategy for colon-specific drug delivery of the subject anti-PESC agents.

Enzyme-Sensitive Drug Delivery Systems—Phloral Technology. Ibekwe et a. “A new concept in colonic drug targeting: A combined pH-responsive and bacterially-triggered drug delivery technology” Aliment. Pharmacol. Ther. 2008, 28, 911-916. reported a novel colonic coating technology which integrated pH-dependent and bacterially-triggered systems into a single layer matrix film. Tablets were film-coated by using a mixture of Eudragit S and biodegradable polysaccharide. Gamma scintigraphy study in human volunteers confirmed the consistent disintegration of these tablets in the colon regardless of feeding status, suggesting that this dual-mechanism coating may overcome the limitation of single trigger systems and improve the colonic drug targeting. Subsequently, Phloral coating technology demonstrated the precise and fail-safe drug release in the colon in both healthy and diseased states. This system consists of an enzyme-sensitive component (natural polysaccharide) and a pH-dependent polymer, where these pH and enzymatic triggers work in a complementary manner to facilitate site-specific release. Even if the dissolution threshold of the pH-dependent polymer is not reached, the enzyme-sensitive component is independently digested by enzymes secreted by colonic microflora. This additional fail-safe mechanism overcomes the limitations of conventional pH-dependent systems. This innovative technology has been validated in clinical studies for consistent drug release with reduced-intra subject variability in patients and healthy subjects.

Ligand/Receptor-Mediated Drug Delivery System. For a more effective local treatment of colonic disease with reduced toxic side effects, ligand/receptor-mediated systems have been explored that increase target specificity via the interaction between targeting ligands on the carrier surface and specific receptors expressed at disease sites. Ligand/receptor-mediated system can be designed using various ligands (e.g., antibodies, peptides, folic acid, and hyaluronic acids) selected based on the functional expression profiles of specific receptors/proteins at the target cells/organs. It can be also combined with pH-dependent systems to maximize its GI stability and site specificity, if needed. Some of the ligands used in colon specific delivery are as described below.

Magnetically-Driven Drug Delivery System. Magnetic microcarriers including magnetic microspheres, magnetic nanoparticles, magnetic liposomes, and magnetic emulsions are emerging novel formulations for controlled and targeted drug delivery. For instance, nanodevices consisting of magnetic mesoporous silica microparticles loaded with anti-PESC agent can be used. The outer surface of the drug-loaded nanoparticles is functionalized with a bulky azo derivative with urea moieties. The nanodevices remained capped at neutral pHs, but a payload release occurs in the presence of sodium dithionite because it reduced the azo bonds in the capping joint. The rate of release can be increased in patients wearing magnetic belts, particularly being more effective when a magnetic field was externally applied to lengthen the retention time in the areas of interest.

In certain embodiments, the anti-PESC agents is formulated for topical administration as part of a bioadhesive formulation—such as for the treatment of perinal disease. Bioadhesive polymers have extensively been employed in transmucosal drug delivery systems and can be readily adapted for use in delivery of the subject anti-PESC agents to the anus or through a supistory to the colon, particularly the areas of lesions and tumor growth. In general terms, adhesion of polymers to tissues may be achieved by (i) physical or mechanical bonds, (ii) primary or covalent chemical bonds, and/or (iii) secondary chemical bonds (i.e., ionic). Physical or mechanical bonds can result from deposition and inclusion of the adhesive material in the crevices of the mucus or the folds of the mucosa. Secondary chemical bonds, contributing to bioadhesive properties, consist of dispersive interactions (i.e., Van der Waals interactions) and stronger specific interactions, which include hydrogen bonds. The hydrophilic functional groups responsible for forming hydrogen bonds are the hydroxyl (—OH) and the carboxylic groups (—COOH). When these materials are incorporated into pharmaceutical formulations, drug absorption by mucosal cells may be enhanced and/or the drug may be released at the site for an extended period of time. Merely to illustrate, the bioadhesive can be a hydrophilic polymer, a hydrogel, a co-polymers/interpolymer complex or a thiolated polymer.
Hydrophilic polymers: These are water-soluble polymers that swell when they come in contact with water and eventually undergo complete dissolution. Systems coated with these polymers show high bioadhesiveness to the mucosa in dry state but the bioadhesive nature deteriorates as they start dissolving. As a result, their bioadhesiveness is short-lived. An example is poly (acrylic acid).
Hydrogels: These are three-dimensional polymer networks of hydrophilic polymers which are cross-linked either by chemical or physical bonds. These polymers swell when they come in contact with water. The extent of swelling depends upon the degree of crosslinking. Examples are polycarbophil, carbopol and polyox.
Co-polymers/Interpolymer complex: A block copolymer is formed when the reaction is carried out in a stepwise manner, leading to a structure with long sequences or blocks of one monomer alternating with long sequences of the other. There are also graft copolymers, in which entire chains of one kind (e.g., polystyrene) are made to grow out of the sides of chains of another kind (e.g., polybutadiene), resulting in a product that is less brittle and more impact-resistant. Hydrogen bonding is a major driving force for interpolymer interactions.
Thiolated polymers (Thiomers): These are hydrophilic macromolecules exhibiting free thiol groups on the polymeric backbone. Based on thiol/disulfide exchange reactions and/or a simple oxidation process disulfide bonds are formed between such polymers and cysteine-rich subdomains of mucus glycoproteins building up the mucus gel layer. So far, the cationic thiomers, chitosan-cysteine, chitosan-thiobutylamidine as well as chitosan-thioglycolic acid, and the anionic thiomers, poly (acylic acid)-cysteine, poly (acrylic acid)-cysteamine, carboxymethylcellulose-cysteine and alginate-cysteine, have been generated. Due to the immobilisation of thiol groups on mucoadhesive basis polymers, their mucoadhesive properties are 2-up to 140-fold improved.

In certain embodiments, the bioadhesive polymer can be selected from poly(acrylic acid), tragacanth, poly(methylvinylether comaleic anhydride), poly(ethylene oxide), methyl-cellulose, sodium alginate, hydroxypropylmethylcellulose, karaya gum, methylethyl cellulose (and cellulose derivatives such as Metolose), soluble starch, gelatin, pectin, poly(vinyl pyrrolidone), poly(ethylene glycol), poly(vinyl alcohol), poly(hydroxyethyl-methacrylate), hydroxypropylcellulose, sodium carboxymethylcellulose or chitosan.

Other suitable bioadhesive polymers are described in U.S. Pat. No. 6,235,313 to Mathiowitz et al., the teachings of which are incorporated herein by reference, and include polyhydroxy acids, such as poly(lactic acid), polystyrene, polyhyaluronic acids, casein, gelatin, glutin, polyanhydrides, polyacrylic acid, alginate, chitosan; polyacrylates, such as poly(methyl methacrylates), poly(ethyl methacrylates), poly butylmethacrylate), poly-(isobutylmethacrylate), poly(hexlmethacrylate), poly(isodecl methacrylate), poly(lauryl methacrylate), poly(phenyl methacrylate), poly(methyl acrylate), poly(isopropyl acrylate), poly(isobutyl acrylate), and poly(octadecl acrylate); polyacrylamides; poly(fumaric-co-sebacic)acid, poly(bis carboxy phenoxy propane-co-sebacic anhydride), polyorthoesters, and copolymers, blends and mixtures thereof.

In certain embodiments, the bioadhesive is an alginate. Alginic acid and its salts associates with sodium and potassium bicarbonate have shown that, after entering a more acidic environment they form a viscous suspension (or a gel) exerting protecting activity over gastric mucosa. These properties are readily adaptable for topical delivery to the anus and colon, including the terminal ileum.

In certain embodiments, the bioadhesive is a bioadhesive hydrogel. Bioadhesive hydrogels are well known in art and suitable hydrogels that be used for delivery of the anti-PESC agents of the present disclosure are described in a wide range of scientific and patent literature on its activity is wide. An exemplary hydrogel formulation is described in Collaud et al. J Control Release. 2007 Nov. 20; 123(3):203-10.

Bioadhesive Microparticle formulations. In certain embodiments, the anti-PESC agent (optionally with other active agents) are formulated into adhesive polymeric microspheres have been selected on the basis of the physical and chemical bonds formed as a function of chemical composition and physical characteristics, such as surface area, as described in detail below. These microspheres are characterized by adhesive forces to mucosa of greater than 11 mN/cm² on mucosal tissue. The size of these microspheres can range from between a nanoparticle to a millimeter in diameter. The adhesive force is a function of polymer composition, biological substrate, particle morphology, particle geometry (e.g., diameter) and surface modification.

Suitable polymers that can be used to form bioadhesive microspheres include soluble and insoluble, biodegradable and nonbiodegradable polymers. These can be hydrogels or thermoplastics, homopolymers, copolymers or blends, natural or synthetic. The preferred polymers are synthetic polymers, with controlled synthesis and degradation characteristics. Most preferred polymers are copolymers of fumaric acid and sebacic acid, which have unusually good bioadhesive properties when administered to the gastrointestinal.

In the past, two classes of polymers have appeared to show useful bioadhesive properties: hydrophilic polymers and hydrogels. In the large class of hydrophilic polymers, those containing carboxylic groups (e.g., poly[acrylic acid]) exhibit the best bioadhesive properties. One could infer that polymers with the highest concentrations of carboxylic groups should be the materials of choice for bioadhesion on soft tissues. In other studies, the most promising polymers were sodium alginate, carboxymethylcellulose, hydroxymethylcellulose and methylcellulose. Some of these materials are water-soluble, while others are hydrogels.

Rapidly bioerodible polymers such as poly[lactide-co-glycolide], polyanhydrides, and polyorthoesters, whose carboxylic groups are exposed on the external surface as their smooth surface erodes, are excellent candidates for bioadhesive drug delivery systems. In addition, polymers containing labile bonds, such as polyanhydrides and polyesters, are well known for their hydrolytic reactivity. Their hydrolytic degradation rates can generally be altered by simple changes in the polymer backbone.

Representative natural polymers include proteins, such as zein, modified zein, casein, gelatin, gluten, serum albumin, or collagen, and polysaccharides, such as cellulose, dextrans, polyhyaluronic acid, polymers of acrylic and methacrylic esters and alginic acid. These are not preferred due to higher levels of variability in the characteristics of the final products, as well as in degradation following administration. Synthetically modified natural polymers include alkyl celluloses, hydroxyalkyl celluloses, cellulose ethers, cellulose esters, and nitrocelluloses.

Representative synthetic polymers include polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Other polymers of interest include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, and polyvinylphenol. Representative bioerodible polymers include polylactides, polyglycolides and copolymers thereof, poly(ethylene terephthalate), poly(butic acid), poly(valeric acid), poly(lactide-co-caprolactone), poly[lactide-co-glycolide], polyanhydrides, polyorthoesters, blends and copolymers thereof.

These polymers can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques.

In some instances, the polymeric material could be modified to improve bioadhesion either before or after the fabrication of microspheres. For example, the polymers can be modified by increasing the number of carboxylic groups accessible during biodegradation, or on the polymer surface. The polymers can also be modified by binding amino groups to the polymer. The polymers can also be modified using any of a number of different coupling chemistries that covalently attach ligand molecules with bioadhesive properties to the surface-exposed molecules of the polymeric microspheres.

One useful protocol involves the “activation” of hydroxyl groups on polymer chains with the agent, carbonyldiimidazole (CDI) in aprotic solvents such as DMSO, acetone, or THF. CDI forms an imidazolyl carbamate complex with the hydroxyl group which may be displaced by binding the free amino group of a ligand such as a protein. The reaction is an N-nucleophilic substitution and results in a stable N-alkylcarbamate linkage of the ligand to the polymer. The “coupling” of the ligand to the “activated” polymer matrix is maximal in the pH range of 9-10 and normally requires at least 24 hrs. The resulting ligand-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfo NHS) to couple the exposed carboxylic groups of polymers to the free amino groups of ligands in a totally aqueous environment at the physiological pH of 7.0. Briefly, EDAC and sulfo-NHS form an activated ester with the carboxylic acid groups of the polymer which react with the amine end of a ligand to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the ligand-polymer complex.

By using either of these protocols it is possible to “activate” almost all polymers containing either hydroxyl or carboxyl groups in a suitable solvent system that will not dissolve the polymer matrix.

A useful coupling procedure for attaching ligands with free hydroxyl and carboxyl groups to polymers involves the use of the cross-linking agent, divinylsulfone. This method would be useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8 and is suitable for transit through the intestine.

Any suitable coupling method known to those skilled in the art for the coupling of ligands and polymers with double bonds, including the use of UV crosslinking, may be used for attachment of bioadhesive ligands to the polymeric microspheres described herein. Any polymer that can be modified through the attachment of lectins can be used as a bioadhesive polymer for purposes of drug delivery or imaging.

Lectins that can be covalently attached to microspheres to render them target specific to the mucin and mucosal cell layer could be used as bioadhesives. Useful lectin ligands include lectins isolated from: Abrus precatroius, Agaricus bisporus, Anguilla anguilla, Arachis hypogaea, Pandeiraea simplicifolia, Bauhinia purpurea, Caragan arobrescens, Cicer arietinum, Codiurn fragile, Datura stramonium, Dolichos biflorus, Erythrina corallodendron, Erythrina cristagalli, Euonymus europaeus, Glycine max, Helix aspersa, Helix pomatia, Lathyrus odoratus, Lens culinaris, Limulus polyphemus, Lysopersicon esculentum, Maclura pomifera, Momordica charantia, Mycoplasma gallisepticum, Naja mocambique, as well as the lectins Concanavalin A, Succinyl-Concanavalin A, Triticum vulgaris, Ulex europaeus I, II and III, Sambucus nigra, Maackia amurensis, Limax fluvus, Homarus americanus, Cancer antennarius, and Lotus tetragonolobus.

The attachment of any positively charged ligand, such as polyethyleneimine or polylysine, to any microsphere may improve bioadhesion due to the electrostatic attraction of the cationic groups coating the beads to the net negative charge of the mucus. The mucopolysaccharides and mucoproteins of the mucin layer, especially the sialic acid residues, are responsible for the negative charge coating. Any ligand with a high binding affinity for mucin could also be covalently linked to most microspheres with the appropriate chemistry, such as CDI, and be expected to influence the binding of microspheres to the gut. For example, polyclonal antibodies raised against components of mucin or else intact mucin, when covalently coupled to microspheres, would provide for increased bioadhesion. Similarly, antibodies directed against specific cell surface receptors exposed on the lumenal surface of the intestinal tract would increase the residence time of beads, when coupled to microspheres using the appropriate chemistry. The ligand affinity need not be based only on electrostatic charge, but other useful physical parameters such as solubility in mucin or else specific affinity to carbohydrate groups.

The covalent attachment of any of the natural components of mucin in either pure or partially purified form to the microspheres would decrease the surface tension of the bead-gut interface and increase the solubility of the bead in the mucin layer. The list of useful ligands would include but not be limited to the following: sialic acid, neuraminic acid, n-acetyl-neuraminic acid, n-glycolylneuraminic acid, 4-acetyl-n-acetylneuraminic acid, diacetyl-n-acetylneuraminic acid, glucuronic acid, iduronic acid, galactose, glucose, mannose, fucose, any of the partially purified fractions prepared by chemical treatment of naturally occurring mucin, e.g., mucoproteins, mucopolysaccharides and mucopolysaccharide-protein complexes, and antibodies immunoreactive against proteins or sugar structure on the mucosal surface.

The attachment of polyamino acids containing extra pendant carboxylic acid side groups, e.g., polyaspartic acid and polyglutamic acid, should also provide a useful means of increasing bioadhesiveness. Using polyamino acids in the 15,000 to 50,000 kDa molecular weight range would yield chains of 120 to 425 amino acid residues attached to the surface of the microspheres. The polyamino chains would increase bioadhesion by means of chain entanglement in mucin strands as well as by increased carboxylic charge.

As used herein, the term “microspheres” includes microparticles and microcapsules (having a core of a different material than the outer wall), having a diameter in the nanometer range up to 5 mm. The microsphere may consist entirely of bioadhesive polymer or have only an outer coating of bioadhesive polymer.

As characterized in the following examples, microspheres can be fabricated from different polymers using different methods. Polylactic acid blank microspheres were fabricated using three methods: solvent evaporation, as described by E. Mathiowitz, et al., J. Scanning Microscopy, 4, 329 (1990); L. R. Beck, et al., Fertil. Steril., 31, 545 (1979); and S. Benita, et al., J. Pharm. Sci., 73, 1721 (1984); hot-melt microencapsulation, as described by E. Mathiowitz, et al., Reactive Polymers, 6, 275 (1987); and spray drying. Polyanhydrides made of bis-carboxyphenoxypropane and sebacic acid with molar ratio of 20:80 P(CPP-SA) (20:80) (Mw 20,000) were prepared by hot-melt microencapsulation. Poly(fumaric-co-sebacic) (20:80) (Mw 15,000) blank microspheres were prepared by hot-melt microencapsulation. Polystyrene microspheres were prepared by solvent evaporation.

In certain embodiments, the composition includes a bioadhesive matrix in which particles (such as nanoparticles) containing the anti-PESC agents are dispersed. In these embodiments, the bioadhesive matrix promotes contact between the mucosa of the gastrointestinal tract and the nanoparticles.

In certain embodiments, the drug-containing particle is a matrix, such as as a bioerodible, bioadhesive matrix. Suitable bioerodible, bioadhesive polymers include bioerodible hydrogels, such as those described by Sawhney, et al., in Macromolecules, 1993, 26:581-587, the teachings of which are incorporated herein by reference. Representative bioerodible, bioadhesive polymers include, but are not limited to, synthetic polymers such as poly hydroxy acids, such as polymers of lactic acid and glycolic acid, polyanhydrides, poly(ortho)esters, polyesters, polyurethanes, poly(butic acid), poly(valeric acid), poly(caprolactone), poly(hydroxybutyrate), poly(lactide-co-glycolide), poly(lactide-co-caprolactone), poly(ethylene-co-maleic anhydride), poly(ethylene maleic anhydride-co-L-dopamine), poly(ethylene maleic anhydride-co-phenylalanine), poly(ethylene maleic anhydride-co-tyrosine), poly(butadiene-co-maleic anhydride), poly(butadiene maleic anhydride-co-L-dopamine) (pBMAD), poly(butadiene maleic anhydride-co-phenylalanine), poly(butadiene maleic anhydride-co-tyrosine), poly(fumaric-co-sebacic)anhydride (P(FA:SA)), poly(bis carboxy phenoxy propane-co-sebacic anhydride) (20:80) (poly(CCP:SA)), as well as blends comprising these polymers; and copolymers comprising the monomers of these polymers, and natural polymers such as alginate and other polysaccharides, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers, blends and mixtures thereof. In general, these materials degrade either by enzymatic hydrolysis or exposure to water in vivo, by surface or bulk erosion.

Particles having an average particle size of between 10 nm and 10 microns are useful in the compositions described herein. In certain embodiments, the particles are nanoparticles, having a size range from about 10 nm to 1 micron, preferably from about 10 nm to about 0.1 microns. In particularly preferred embodiments, the particles have a size range from about 500 to about 600 nm. The particles can have any shape but are generally spherical in shape.

The compositions described herein contain a monodisperse plurality of nanoparticles. Preferably, the method used to form the nanoparticles produces a monodisperse distribution of nanoparticles; however, methods producing polydisperse nanoparticle distributions can be used. If the method does not produce particles having a monodisperse size distribution, the particles are separated following particle formation to produce a plurality of particles having the desired size range and distribution.

Nanoparticles useful in the compositions described herein can be prepared using any suitable method known in the art. Common microencapsulation techniques include, but are not limited to, spray drying, interfacial polymerization, hot melt encapsulation, phase separation encapsulation (spontaneous emulsion microencapsulation, solvent evaporation microencapsulation, and solvent removal microencapsulation), coacervation, low temperature microsphere formation, and phase inversion nanoencapsulation (PIN). A brief summary of these methods is presented below.

Spray Drying. Methods for forming microspheres/nanospheres using spray drying techniques are described in U.S. Pat. No. 6,620,617, to Mathiowitz et a. In this method, the polymer is dissolved in an organic solvent such as methylene chloride or in water. A known amount of one or more active agents to be incorporated in the particles is suspended (in the case of an insoluble active agent) or co-dissolved (in the case of a soluble active agent) in the polymer solution. The solution or dispersion is pumped through a micronizing nozzle driven by a flow of compressed gas, and the resulting aerosol is suspended in a heated cyclone of air, allowing the solvent to evaporate from the microdroplets, forming particles. Microspheres/nanospheres ranging between 0.1-10 microns can be obtained using this method.

Interfacial Polymerization. Interfacial polymerization can also be used to encapsulate one or more active agents. Using this method, a monomer and the active agent(s) are dissolved in a solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

Hot Melt Microencapsulation. Microspheres can be formed from polymers such as polyesters and polyanhydrides using hot melt microencapsulation methods as described in Mathiowitz et al., Reactive Polymers, 6:275 (1987). In this method, the use of polymers with molecular weights between 3-75,000 daltons is preferred. In this method, the polymer first is melted and then mixed with the solid particles of one or more active agents to be incorporated that have been sieved to less than 50 microns. The mixture is suspended in a non-miscible solvent (like silicon oil), and, with continuous stirring, heated to 5.degree. C. above the melting point of the polymer. Once the emulsion is stabilized, it is cooled until the polymer particles solidify. The resulting microspheres are washed by decanting with petroleum ether to give a free-flowing powder.

Phase Separation Microencapsulation. In phase separation microencapsulation techniques, a polymer solution is stirred, optionally in the presence of one or more active agents to be encapsulated. While continuing to uniformly suspend the material through stirring, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the active agent(s) in a droplet with an outer polymer shell.

Spontaneous Emulsion Microencapsulation. Spontaneous emulsification involves solidifying emulsified liquid polymer droplets formed above by changing temperature, evaporating solvent, or adding chemical cross-linking agents. The physical and chemical properties of the encapsulant, as well as the properties of the one or more active agents optionally incorporated into the nascent particles, dictates suitable methods of encapsulation. Factors such as hydrophobicity, molecular weight, chemical stability, and thermal stability affect encapsulation.

Solvent Evaporation Microencapsulation. Methods for forming microspheres using solvent evaporation techniques are described in E. Mathiowitz et al., Scanning Microscopy, 4:329 (1990); L. R. Beck et al., Fertil. Steril., 31:545 (1979); L. R. Beck et al Am J Obstet Gynecol 135(3) (1979); S. Benita et al., Pharm. Sci., 73:1721 (1984); and U.S. Pat. No. 3,960,757 to Morishita et al. The polymer is dissolved in a volatile organic solvent, such as methylene chloride. One or more active agents to be incorporated are optionally added to the solution, and the mixture is suspended in an aqueous solution that contains a surface active agent such as poly(vinyl alcohol). The resulting emulsion is stirred until most of the organic solvent evaporated, leaving solid microspheres/nanospheres. This method is useful for relatively stable polymers like polyesters and polystyrene. However, labile polymers, such as polyanhydrides, may degrade during the fabrication process due to the presence of water. For these polymers, some of the following methods performed in completely anhydrous organic solvents are more useful.

Solvent Removal Microencapsulation. The solvent removal microencapsulation technique is primarily designed for polyanhydrides and is described, for example, in WO 93/21906 to Brown University Research Foundation. In this method, the substance to be incorporated is dispersed or dissolved in a solution of the selected polymer in a volatile organic solvent, such as methylene chloride. This mixture is suspended by stirring in an organic oil, such as silicon oil, to form an emulsion. Microspheres that range between 1-300 microns can be obtained by this procedure. Substances which can be incorporated in the microspheres include pharmaceuticals, pesticides, nutrients, imaging agents, and metal compounds.

Coacervation. Encapsulation procedures for various substances using coacervation techniques are known in the art, for example, in GB-B-929 406; GB-B-929 40 1; and U.S. Pat. Nos. 3,266,987, 4,794,000, and 4,460,563. Coacervation involves the separation of a macromolecular solution into two immiscible liquid phases. One phase is a dense coacervate phase, which contains a high concentration of the polymer encapsulant (and optionally one or more active agents), while the second phase contains a low concentration of the polymer. Within the dense coacervate phase, the polymer encapsulant forms nanoscale or microscale droplets. Coacervation may be induced by a temperature change, addition of a non-solvent or addition of a micro-salt (simple coacervation), or by the addition of another polymer thereby forming an interpolymer complex (complex coacervation).

Low Temperature Casting of Microspheres. Methods for very low temperature casting of controlled release microspheres are described in U.S. Pat. No. 5,019,400 to Gombotz et al. In this method, a polymer is dissolved in a solvent optionally with one or more dissolved or dispersed active agents. The mixture is then atomized into a vessel containing a liquid non-solvent at a temperature below the freezing point of the polymer-substance solution which freezes the polymer droplets. As the droplets and non-solvent for the polymer are warmed, the solvent in the droplets thaws and is extracted into the non-solvent, resulting in the hardening of the microspheres.

Phase Inversion Nanoencapsulation (PIN). Nanoparticles can also be formed using the phase inversion nanoencapsulation (PIN) method, wherein a polymer is dissolved in a “good” solvent, fine particles of a substance to be incorporated, such as a drug, are mixed or dissolved in the polymer solution, and the mixture is poured into a strong non-solvent for the polymer, to spontaneously produce, under favorable conditions, polymeric microspheres, wherein the polymer is either coated with the particles or the particles are dispersed in the polymer. See, e.g., U.S. Pat. No. 6,143,211 to Mathiowitz, et a. The method can be used to produce monodisperse populations of nanoparticles and microparticles in a wide range of sizes, including, for example, about 100 nanometers to about 10 microns.

Advantageously, an emulsion need not be formed prior to precipitation. The process can be used to form microspheres from thermoplastic polymers.

Sequential Phase Inversion Nanoencapsulation (sPIN). Multi-walled nanoparticles can also be formed by a process referred to herein as “sequential phase inversion nanoencapsulation” (sPIN). This process is described in detail below in Section IV. sPIN is particularly suited for forming monodisperse populations of nanoparticles, avoiding the need for an additional separations step to achieve a monodisperse population of nanoparticles.

Perianal Applications—Topical and Suppositories. In certain embodiments, the anti-PESC agents and (optionally) the ESO Regenerative agents of the invention can be used as part of a treatment for perianal symptoms include perianal erythema, abscesses, ulcers and perianal fissures or fistulas.

In certain embodiments, the anti-PESC agents and (optionally) the ESO Regenerative agents of the invention can be used as part of a treatment for perianal disease associated with Crohn's Disease.

In certain embodiments, the anti-PESC agents and (optionally) the ESO Regenerative agents of the invention can be formulated for an internal or perianal application, such as in a dosage form selected from a rectal suppository, an enema, a cream, a lotion, a gel, an ointment, an emulsion, a solution, a suspension, an elixir, a tincture, a paste, a foam, an aerosol, a spray and an application syringe.

In certain embodiments, the invention relates to a pharmaceutical composition including an anti-PESC agent and/or a ESO Regenerative agent formulated suppository.

In a further aspect, an anti-PESC agent and/or a ESO Regenerative agent is formulated for topical application to skin. Topical formulations may include powders, sprays, ointments, pastes, creams, lotions, gels, solutions, patches, and liposomal preparations.

Optionally, the anti-PESC agents and/or the ESO Regenerative agents of the invention can be formulationed with one or more additional agents selected from the group consisting of antibiotic, vasoconstrictors, analgesics or local anesthtic, cytoprotective agents, muscle relaxants and sodium channel blocker, antipruritic agents, immunomodulators, cytotoxins, anti-inflammatory agents, and a combination thereof.

For instance, the sodium channel blocker can be procaine, benzocaine, chloroprocaine, cocaine, cyclomethycaine, dimethocaine/larocaine, piperocaine, propoxycaine, procaine/novocaine, proparacaine, tetracaine/amethocaine, lidocaine, articaine, bupivacaine, cinchocaine/dibucaine, etidocaine, levobupivacaine, lidocaine/lignocaine, mepivacaine, prilocaine, ropivacaine or trimecainelidocaine. In certain embodiments, the sodium channel blocker is lidocaine.

For instance, the cytoprotective agent can be sucralfate.

Capsaicin and other capsaicinoids may also be added to the composition for their analgesic properties.

For instance, the muscle relaxant is a calcium channel blocker, such as amlodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nilmodipine, nisoldipine, nitrendipine, pranidipine, verapamil, nitroglycerin, sildenafil, or diltiazem.

In certain embodiments, the formulation includes an additional active agent(s) selected from a corticosteroid, mesalamine, balsalazide, olsalazine, diclofenac, azathioprine, mercaptopurine, cyclosporine, methotrexate, ciprofloxacin, metronidazole, lidocaine, pramoxine and combinations thereof.

In certain embodiments, the formulation includes an anti-inflammatory agent selected from the group consisting of salicylic acid, indomethacin, sodium indomethacin trihydrate, salicylamide, naproxen, colchicine, fenoprofen, sulindac, diflunisal, diclofenac, indoprofen and sodium salicylamide. According to still further embodiment, the anti-inflammatory agent is salicylic acid.

In certain embodiments, the formulation includes an antipruritic agent selected from the group comprising corticosteroid, camphor, juniper tar, menthol and a combination thereof. According to a certain embodiment, the corticosteroid is hydrocortisone. According to some embodiments, the antipruritic agent is present in the topical composition in an amount ranging from about 0.1% (w/w) to about 5% (w/w).

In certain embodiments, the formulation includes an anesthetic agents such as pramoxine, procaine, lidocaine, tetracaine, dibucaine, prilocaine, phenacaine, benzyl alcohol, benzocaine, diperodon, dyclonine, dimethisoquin and combinations thereof.

In certain embodiments, the formulation includes a vasoconstrictorsuch as amphetamines, antihistamines, methylphenidate, mephedrone, oxymetazoline, phenylephrine, pseudoephedrine, psilocybin, phenylephrine hydrochloride, ephedrine sulphate, epinephrine, epinephrine hydrochloride, tetrahydrozoline hydrochloride, and combinations thereof.

In certain embodiments, the formulation includes an antipruritic agents, such as a corticosteroid, camphor, juniper tar and menthol. The non-limiting examples of corticosteroids include hydrocortisone, fluocinolone, flurandrenolide, triamcinolone, fluticasone, and desonide. Antipruritic agents may further comprise corticosteroids such as tetrahydrocortisol, prednisone; prednisolone, fludrocortisone, 11-desoxycortisol, cortisone, corticosterone, paramethasone, betamethasone, dexamethasone, desoxycorticosterone acetate, desoxycorticosterone pivalate, fludrocortisone acetate, cortisol acetate, cortisol cypionate, cortisol sodium phosphate, cortisol sodium succinate, beclopmethasone dipropionate, betamethasone, betamethasone sodium phosphate and acetate, betamethasone dipropionate, betamethasone valerate, betamethasone benzoate, cortisone acetate, dexamethasone, dexamethasone sodium phosphate, dexamethasone acetate, fuprednisolone, meprednisone, methylprednisolone, methylprednisolone acetate, methylprednisolone sodium succinate, paramethasone acetate, prednisolone, prednisolone acetate, prednisolone sodium phosphate, prednisolone sodium succinate, prednisolone tebutate, prednisone, triamcinolone acetonide, triamcinolone diacetate, triamcinolone hexacotonide, desoximetasone, flumethasone pivalate, fluocinolone acetonide, fluocinonide, fluorometholone, halcinonide, and medrysone.

In certain embodiments, the composition includes an additional active agent(s) selected from pramoxine, phenylephrine, hydrocortisone, salicylic acid, nitroglycerine, sildenafil, procaine, lidocaine, tetracaine, dibucaine, prilocaine, phenacaine, benzyl alcohol, benzocaine, diperodon, dyclonine, dimethisoquin, epinephrine, tetrahydrozoline hydrochloride, an amphetamine, an antihistamine, methylphenidate, mephedrone, oxymetazoline, pseudoephedrine, psilocybin, ephedrine sulfate or their salts and combinations thereof.

Antibiotics such as metronidazole may also be used as additives to the composition.

Formulations for suppository or perianal applications of the composition may further comprise one or more carriers or excipients suitable for topical application or suitable for formulation of a suppository. Examples of carriers or excipients include but are not limited to oil, including vegetable oils such as olive oil, sesame oil or nut oils, emulsions of water and oil, petrolatum, mineral oil, paraffins, microcrystalline wax, ceresine, wool fat, beeswax, macrogols 200, 300, 400, emulsifying wax, cetrimide, synthetic hydrocarbons, zinc oxide, alcohol, cellulose ethers, carbomer in water and water-alcohol mixtures, cocoa butter, polyethylene glycol, glycerin and gelatin.

The pharmacologically acceptable excipients usable in the formulation as a gel, cream, enema, or rectal suppository, are selected from the group consisting of glycerine, Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, potassium acetate, and mixtures of two or more components thereof. Other pharmaceutically acceptable excipients typically employed in the preparation of a gel, cream, or rectal suppository or solutions can be used in the present invention.

In a particular embodiment the carrier is petroleum jelly.

Formulations for suppository or perianal applications of the composition may also include additives. The additives may be preservatives, buffers, propellants, colourants, fragrances, emulsifiers, and fat soluble anti-oxidant vitamins such as vitamins A, D, and E which can assist in wound repair and healing.

Where the administration route is rectal and the preferred formulations are a rectal enema, a rectal suppository, a rectal a foam, a cream, a lotion, or a gel.

Another aspect of the invention provides a kit comprising the anti-PESC agent and/or the ESO Regenerative agent (as a pharmaceutical formulation) and an applicator device suitable for storage and application of the composition to the anorectal region.

For instance, the applicator device can be selected from the group consisting of a single use wipe, a syringe, a dropper, a spray dispenser, a compressible bottle or tube, a spatula, a suppository insertion tube, an extrusion tube, and an inflatable member.

A topical composition of the present invention may further include an astringent. As used herein, an “astringent” refers to a substance that causes tissue (e.g., a hemorrhoidal) to contract and can optionally arrest secretion or control bleeding from tissue. Astringents which are suitable for use in the invention include, e.g., alum, tannic acid, calamine, witch hazel, zinc oxide, or a combination thereof. Suitable amounts of such astringents in the composition may be readily ascertained by one of ordinary skill in the art, and may range, for example, between about 2% (w/w) and about 50% (w/w).

A topical composition of the present invention may further include a keratolytic agent. As used herein, a “keratolytic agent” refers to a substance that causes desquamation (loosening) and debridement or sloughing of the surface cells of the epidermis. Typically, the keratolytic agent used in the compositions of the present invention are pharmaceutically acceptable for topical use in humans. Suitable keratolytic agents include, but are not limited to, alcloxa, resorcinol, or a combination thereof. Suitable amounts of such keratolytic agents in the composition may be readily ascertained by one of ordinary skill in the art, and may range, for example, between about 0.1% (w/w) and about 5% (w/w).

Antibiotics for use in the invention are typically those suitable for topical application. The antibiotic(s) may be classified in one or more of the following groups: penicillins, cephalosporins, carbepenems, beta-lactam antibiotics, aminoglycosides, amphenicols, ansamycins, macrolides, lincosamides, glycopeptides, polypeptides, tetracylines, chloramphenicol, quinolones, fucidins, sulfonamides, sulfones, nitrofurans, diaminopyrimidines, trimethoprims, rifamycins, oxalines, streptogramins, lipopeptides, ketolides, polyenes, azoles, and echinocandins.

Specific examples of antibiotics which are suitable for use in the invention include: amikacin, aminosidine, paromomycin, chloramphenicol, ciprofloxacin, clindamycin, colistimethate-sodium, colistin, enfuvirtid, enoxacin, erythromycin, flucloxacillin, fosfomycin, fusafungin, gentamicin, levofloxacin, linezolid, mefloquin, metronidazol, mezlocillin, moxifloxacin, mupirocin, norfloxacin, ofloxacin, oxacillin, penicillin G, penicillin V, phenoxymethylpenicillin, phenoxymethylpenicillin-benzathin, pipemidinic acid, piperacillin, piperacillin+tazobactam, proguanil, propicillin, pyrimethamine, retapamulin, rifaximin, roxithromycin, sodium sulfacetamide, sulbactam, sulbactam+ampicillin, sulfadiazine, spiramycin, sultamicillin, tazobactam+piperacillin, teicoplanin, telithromycin, tigecyclin, vancomycin and combinations thereof.

Antiseptics which are suitable for use in the invention include, e.g., triclosan, phenoxy isopropanol, chlorhexidine gluconate, povidone iodine, and any combination thereof.

Antioxidative compounds may also be included in the composition, in particular the antioxidative compounds collectively termed catechins. These include for example, epicatechin, epicatechin gallate, epigallocatechin gallate, and gallocatechin, as well as stereoisomers and enantiomers of these compounds and combinations thereof. Such compounds may be provided as synthetic compounds or in the forms of mixtures as components of plant extracts, in particular green tea extracts. Botanical products and extracts include those derived from peppermint, ginger horseradish, yarrow, chamomile, rosemary, capsicum, aloe vera, tea tree oil (melaleuca oil), among many others.

A topical composition of the present invention may further include protectant active ingredients. The protectant active ingredients can be selected from the group consisting of aluminum hydroxide gel, cocoa butter, aqueous solution of glycerin, hard fat, kaolin, lanolin, mineral oil, petrolatum, topical starch, white petrolatum, cod liver, shark liver oil, and a combination thereof. The protectant active ingredient and the dosage thereof is dependent upon the particular condition to be treated, the pharmaceutical active agents present in the composition and other factors evident to those skilled in the art.

A topical composition of the present invention may include one or more of the following additional ingredients: emulsifiers (e.g. anionic, cationic or nonionic), chelating agents, colorants, emollients, fragrances, humectants, lubricants, moisturizers, preservatives, skin penetration enhancers, stabilizers, thickeners, and viscosity modifiers.

The topical composition is preferably in a form suitable for direct application to the colon, rectum, anorectum, perianal region or anal canal. Suitable forms include an enema, suppository, ointment, lotion, gel, foam or cream. Preferred forms include ointment or enema. The ointment, lotion, gel or cream forms may be used to treat conditions affecting the perianal region and anorectum including perianal Crohn's disease and conditions arising following a hermorrhoidectomy. The suppository, foam or enema forms may be used to treat conditions affecting the colon or rectum including inflammatory bowel disease (Crohn's disease or ulcerative colitis), radiation proctitis, idiopathic proctocolitis or post-surgical pouchitis in a surgically constructed ileal J-pouch.

### VI. Combination Therapies—Other Agents

In certain embodiments, the anti-PESC agent can be administered conjointly with one or more agents that have other beneficial local activities in gastrointestinal tract.

In certain embodiments, the anti-PESC agent is conjointly administered with an anti-inflammatory agent selected from an IL-1 inhibitor, an IL-1 receptor (IL-1R) inhibitor, an IL-6 inhibitor, an IL-6 receptor (IL-6R) inhibitor, a NLRP3 inhibitor, a TNF inhibitor, an IL-8 inhibitor, an IL-18 inhibitor, an inhibitor of natural killer cells, or combinations thereof. In some embodiments, the anti-inflammatory agent is a nucleic acid, an aptamer, an antibody or antibody fragment, an inhibitory peptide, or a small molecule.

In certain embodiments, the anti-PESC agent is conjointly administered with an an NLRP3 inhibitor. In some embodiments, the NLPR3 inhibitor is an anti-sense oligonucleotide against NLPR3, colchicine, MCC950, CY-09, ketone metabolite beta-hydroxubutyrate (BHB), a type I interferon, resveratrol, arglabin, CB2R, Glybenclamide, Isoliquiritigenin, Z-VAD-FMK, or microRNA-223.

In certain embodiments, the anti-PESC agent is conjointly administered with a TNF inhibitor. In some embodiments, the TNF inhibitor is an anti-sense oligonucleotide against TNF, infliximab, adalimumab, certolizumab pegol, golimumab, etanercept (Enbrel), thalidomide, lenalidomide, pomalidomide, a xanthine derivative, bupropion, 5-HT2A agonist or a hallucinogen.

In certain embodiments, the anti-PESC agent is conjointly administered with an IL-18 inhibitor. In some embodiments, the IL-18 inhibitor is selected from the group consisting of: anti-sense oligonucleotides against IL-18, IL-18 binding protein, IL-18 antibody, NSC201631, NSC61610, and NSC80734.

In certain embodiments, the anti-PESC agent is conjointly administered with an inhibitor of natural killer cells. In some embodiments, the inhibitor of natural killer cells is an antibody targeting natural killer cells.

In certain embodiments, the anti-PESC agent is conjointly administered with methotrexate.

In certain embodiments, the anti-PESC agent is conjointly administered with arhalofenate.

In certain embodiments, the anti-PESC agent is conjointly administered with an IL-10 inhibitor.

a. STAT3 Inhibitors

In certain embodiments, the anti-PESC agent is conjointly administered with a STAT3 inhibitor.

In one embodiment, the STAT3 inhibitor is Stattic. Stattic is nonpeptidic small molecule that potently inhibits STAT3 activation and nuclear translocation with IC50 of 5.1 μM in cell-free assays, highly selectivity over STAT1.

Non-limiting examples of STAT3 inhibitors include BP-1-102, S31-M2001, STA-21, S31-201, Galiellalactone, a polypeptide having the sequence PY*LKTK (where Y* represents phosphotyrosine), and a polypeptide having the sequence Y*LPQTV (where Y* represents phosphotyrosine). Additional non-limiting examples of STAT3 inhibitors are described in Yue and Turkson Expert Opin Investig Drugs. 2009 January; 18(1): 45-56, the entire content of which is incorporated herein by reference.

Other STAT3 inhibitors include: E1: 4_-Bromo-phenyl-2-N-aminoacyl-1 1-dioxide-benzo [b]thiophene; E2: 4_-bromo-2-N-(4-fluorophenyl) alanyl-1,1-dioxide, benzo [b] thiophene; E3: 4_-bromo-benzo 2-N-(4-methoxyphenyl) alanyl-1,1-dioxide [b] thiophene; E4: 4_-bromo-2-N-aminoacyl-p-tolyl-1,1-oxidation benzo [b] thiophene; E5: 4_-bromo-2-N-(4-chlorophenyl) alanyl-1,1-dioxide, benzo [b] thiophene; E6: 4_-bromo-2-N -benzo (3-chlorophenyl) alanyl-1,1-dioxide [b] thiophene; E7 4_-bromo-2-N-(2-chlorophenyl) alanyl-1,1-dioxide benzo [b] thiophene; E8: 4_-bromo-2-N-(3-chloro-4-fluorophenyl) alanyl-1,1-dioxide, benzo [b] thiophene; E9: 4_-chloro-2-N-aminoacyl-phenyl-1,1-dioxide, benzo [b]thiophene; E10: 5_-bromo-phenyl-2-N-aminoacyl-1,1-dioxide, benzo [b] thiophene; EII: 6_bromo-phenyl-2-N-aminoacyl-1,1-dioxide, benzo [b] thiophene; E12: 2-N-aminoacyl-phenyl-1,1-dioxide, benzo [b] thiophene; E13: 5_-nitro-phenyl-2-N-Acyl-1,1-dioxide, benzo [b] thiophene; E14: 5_-bromo-n-butyl-2-N-aminoacyl-1,1-dioxide, benzo [b] thiophene; E15: 5_bromo-2-N-aminoacyl-t-butyl-1,1-dioxide, benzo [b] thiophene; E16: 5_-bromo-2-N-isopropyl-alanyl-1,1-benzo [b] dioxide thiophene; E17: 5_-bromo-2-N-cyclohexyl-alanyl-1,1-benzo [b] thiophene dioxide; E18: 5_-bromo-2-N-[(3s, 5s, 7s)-1-adamantyl]-1,1-aminoacyl dioxide benzo [b] thiophene; E19: 4_-bromo-benzo-2-N-benzyl-aminoacyl-1,1-dioxide [b] thiophene; E20: 4_-bromo-2-N-(4-bromophenethyl) benzo-1,1-dioxide aminoacyl [b] thiophene; E21: 5_-bromo-2-N-(4-phenoxy-phenyl) amino-benzo-1,1-dioxide group [b] thiophene; E22: 5_-bromo-2-N-[4-(I-piperidinyl-carbonyl) phenyl]-1,1-aminoacyl dioxide benzo [b] thiophene; E23: 5_-bromo-2-N-[4-(4-morpholin-ylcarbonyl) phenyl] carboxamido-1,1-dioxide, benzo [b] thiophene; E24: 5_-bromo-2-N-[4-(N-methyl-N-phenyl) carbamoyl] phenyl-carboxamido-1,1-dioxide, benzo [b] thiophene; E25: 4_bromo-2-p-tolyl-carboxy-1,1-dioxide [b] thiophene; E26: 5_bromo-2-N, N-diethyl-1,1-dioxide aminoacyl benzothienyl; E27: 5_-bromo-2-(I-pyrrolyl) carbonyl-1,1-dioxide, benzo [b] thiophene; E28: 5_-bromo-2-(1-piperidyl) carbonyl-1,1-dioxide, benzo [b] thiophene; E29: 5_-bromo-2-(2-methyl-piperidine yl) carbonyl-1,1-dioxide, benzo [b] thiophene; E30: benzo 5_-bromo-2-(3-methyl-1-piperidinyl) carbonyl-1,1-dioxide [b] thiophene; E31: 5-bromo-2-morpholino-carbonyl-1,1-dioxide, benzo [b] thiophene; E32: 5_-bromo-2-(4-ethyl-1-piperazinyl) carbonyl-1, 1-dioxide-benzo [b] thiophene; E33: 5_-bromo-2-(N-methyl-N-phenyl) benzo-1,1-dioxide aminoacyl [b] thiophene; E34: 4_bromo-2-(I-piperidinyl) carbonyl-1,1-dioxide, benzo [b] thiophene; E35: 5_trifluoromethyl-2-(I-piperidinyl) carbonyl-1,1-dioxide benzo [b] thiophene; E36: 4_-bromo-2-methoxycarbonyl-1,1-dioxide, benzo [b] thiophene; E37: 2_methoxycarbonyl-1,1-oxidation benzo [b] thiophene; E38: benzo 5_acetamido-2-N-phenyl-1,1-dioxide aminoacyl [b] thiophene; E39: 5_benzoylamino-2-N -aminoacyl phenyl-1,1-dioxide, benzo [b] thiophene; E40: 5_of Methylbenzamido-2-N-aminoacyl-phenyl-1,1-dioxide, benzo [b] thiophene; E41: 5_Trifluoromethyl-benzoyl-phenylcarbamoyl group -2-N-acyl-1,1-dioxide, benzo [b]thiophene; E42: 5_p-chlorobenzoyl-N-phenylcarbamoyl group an acyl-2-1,1-benzo [b]thiophene dioxide; E43: 5_-cyclohexyl-carboxamido-2-N-phenyl-aminoacyl-1,1-dioxide, benzo [b] thiophene; or E44: 5_benzamido-2-(I-piperidinyl) carbonyl 1,1-dioxide, benzo [b] thiophene.

b. IL-6 Inhibitors

In certain embodiments, the anti-PESC agent is conjointly administered with an IL-6 inhibitor, such agent that binds to IL-6 or the IL-6 receptor and prevents the interaction of those two molecules, or which inhibits signal transduction resulting from IL-6 binding to IL-6R containing receptor complexes. These include anti-IL-6 antibodies and antibody mimetic, anti-IL-6 receptor antibodies and antibody mimetics and small molecules, as well as nucleic acids which down-regulate IL-6 mediated signal transduction.

Exemplary agents targeting IL-6 or the IL-6 receptor include such as tocilizumab (Actemra), siltuximab (Sylvant), sarilumab, ALX-0061, sirukumab, MED15117, clazakizumab, and olokizumab. Tocilizumab is an example of an antibody directed against the IL6-receptor, siltuximab is directed against IL-6 itself.

In some embodiments, the anti-inflammatory agent comprises an IL-6 inhibitor. In some embodiments, the IL-6 inhibitor is an anti-sense oligonucleotide against IL-6, siltuximab, sirukumab, clazakizumab, olokizumab, elsilimomab, IG61, BE-8, CNT0328 PGE1 and its derivatives, PG12 and its derivatives, or cyclophosphamide.

In some embodiments, the anti-inflammatory agent comprises an IL-6R inhibitor. In some embodiments, the IL-6R inhibitor is an IL-6R antagonist. In some embodiments, the IL-6R inhibitor is an anti-sense oligonucleotide against IL-6R, tocilizumab, sarilumab, PM1, AUK 12-20, AUK64-7, AUK146-15, MRA, or AB-227-NA.

c. IL-8 Inhibitors

In certain embodiments, the anti-PESC agent is conjointly administered with an IL-8 inhibitor. In some embodiments, the IL-8 inhibitor is an anti-sense oligonucleotides against IL-8, HuMab-10F8, repertaxin, Curcumin, Antileukinate, Macrolide, or a trifluoroacetate salt.

d. IL-1 Inhibitors

In some embodiments, the anti-inflammatory agent comprises an IL-1 inhibitor. In some embodiments, the IL-1 inhibitor is an IL-1a inhibitor. In some embodiments, the IL-1a inhibitor is an anti-sense oligonucleotide against IL-1a, MABpI, or sIL-1RI. In some embodiments, the IL-1 inhibitor is an IL-1b inhibitor. In some embodiments, the IL-1b inhibitor is an anti-sense oligonucleotides against IL-1b, canakinumab, diacerein, gevokizumab, LY2189102, CYT013, sIL-IRII, VX-740, or VX-765. In some embodiments, the IL-I inhibitor is suramin sodium, methotrexate-methyl-d3, methotrexate-methyl-d3 dimethyl ester, or diacerein.

In some embodiments, the anti-inflammatory agent comprises an IL-1R inhibitor. In some embodiments, the IL-1R inhibitor is an IL-1R antagonist. In some embodiments, the IL-1R inhibitor is an anti-sense oligonucleotide against IL-1R, anakinra, Rilonacept, MEDI-8968, sIL-IRI, EBI-005, interleukin-I receptor antagonist (IL-1RA), or AMG108.

### VII. Examples

The following Examples section provides further details regarding examples of various embodiments. It should be appreciated by those of skill in the art that the techniques disclosed in the examples that follow represent techniques and/or compositions discovered by the inventors to function well. However, those of skill in the art should, in light of the present disclosure, appreciate that many changes can be made in the specific embodiments which are disclosed and still obtain a like or similar result without departing from the spirit and scope of the disclosure. These examples are illustrations of the methods and systems described herein and are not intended to limit the scope of the disclosure. Non-limiting examples of such include but are not limited to those presented below.

### Example 1: Ground State Culture Vs Organoids: Differential Fate of Cloned Human Intestinal Stem Cells

The cloning of pluripotent embryonic stem cells (ESC) unleashed their potential for reverse genetics and deciphering pathways of development and disease. With few exceptions adults stem cells are not maintained in an immature, clonogenic state like ESCs but rather as regenerative “organoids” whose absolute relationship to stem cells remains largely unexplored. A direct comparison of the fate of cloned human intestinal stem cells (ISCs) in “ground state” culture versus organoids reveals profound differences in clonogenicity, stem cell gene expression profiles, and major signaling pathways dictating self-renewal, intrinsic immortality, and lineage specification.

The cloning of pluripotent embryonic stem cells (ESC) in the laboratories of Martin Evans and Gail Martin unleashed the potential of these cells for genetic manipulation, the generation of mice bearing precisely engineered alleles, and deciphering developmental and disease pathways[1]. Moreover, this cloning resolved simmering debates as to the relationship between these inner cell mass (ICM)-derived cells and those from early embryos or genital ridges that Leroy Stevens showed gave rise to the embryonal carcinoma (EC) cells of teratocarcinomas[2]. Adult stem cells of regenerative epithelia are defined as tissue-specific, immature cells with the capacities of lineage-restricted differentiation and long-term self-renewal potential[3], but remain conceptually, at least, at the precloning days of ESCs. Therefore the development of tools akin to those for ESCs that capture and maintain adult stem cells in their elemental state will essential for both regenerative medicine and stem cell-based drug discovery for multiple diseases.

Following the localization by Cheng and her colleagues of stem cell activity to the crypt base of intestinal epithelia [4], analyses of discrete markers by lineage tracing and immunofluorescence have highlighted the importance of Lgr5+ cells in the crypt base, BMI1+‘plus 4” crypt cells, as well as an exotic program of transformations between not only these two cells but a host of other, quasidifferentiated cells in the crypt [5-8]. Whether a similar interplay exists between ESCs and partially differentiated progeny exist is unclear, but nevertheless the cloning efforts of Evans and others captured a “functional” pluripotent stem cell with unlimited proliferative potential that could reconstitute a blastocyst and contribute to all tissues including germ cells during embryonic development. Efforts to functionalize adult stem cells have taken many routes, but certainly the dominant method for representing intestinal stem cells (ISCs) ex vivo has been as so-called “organoids” or “mini-guts” [9]. Despite the near universal use of organoids in stem cell biology, relatively little is known about how they relate to stem cells or model differentiation or disease. For instance, while Lgr5+ ISC-initiated organoids are regenerative, the growth kinetics of these spheroids is slow and the vast majority of cells in an organoid cease to express Lgr5. Whether this Lgr5-negative majority has converted to Bmi1+ cells or progressed to more differentiated intestinal cell lineages is unknown, but this predominant, non-ISC population obscures any molecular analysis of Lgr5+ ISCs in these structures. What is clear from the growth dynamics of organoid passaging is that most organoid cells have lost the critical stem cell property of “clonogenicity”—the ability of a single organoid cell to generate a new organoid [10].

In contrast, using the ground-state ISC culture system [11-13], the inventors showed that ISCs are highly clonogenic (>70%), intrinsically immortal, and can be maintained as undifferentiated cells in vitro that retain the potential of regio-specific multipotency. These radically different modes of perpetuating ISCs— as uniform ground state stem cells versus organoids—invokes the analogy between Evans' cloned ESCs and Stevens' teratomas [1,2]. In particular, ground state ISCs possess a clonogenicity that approaches unity, express multiple stem cell genes (OLFM4, CD133, LGR5, NR5A2, ID2, LRIG1, EPHB2, ASCL2, but not BMI1), and yet show a very precise and stable differentiation program consistent with their origin along the gastrointestinal tract [11].

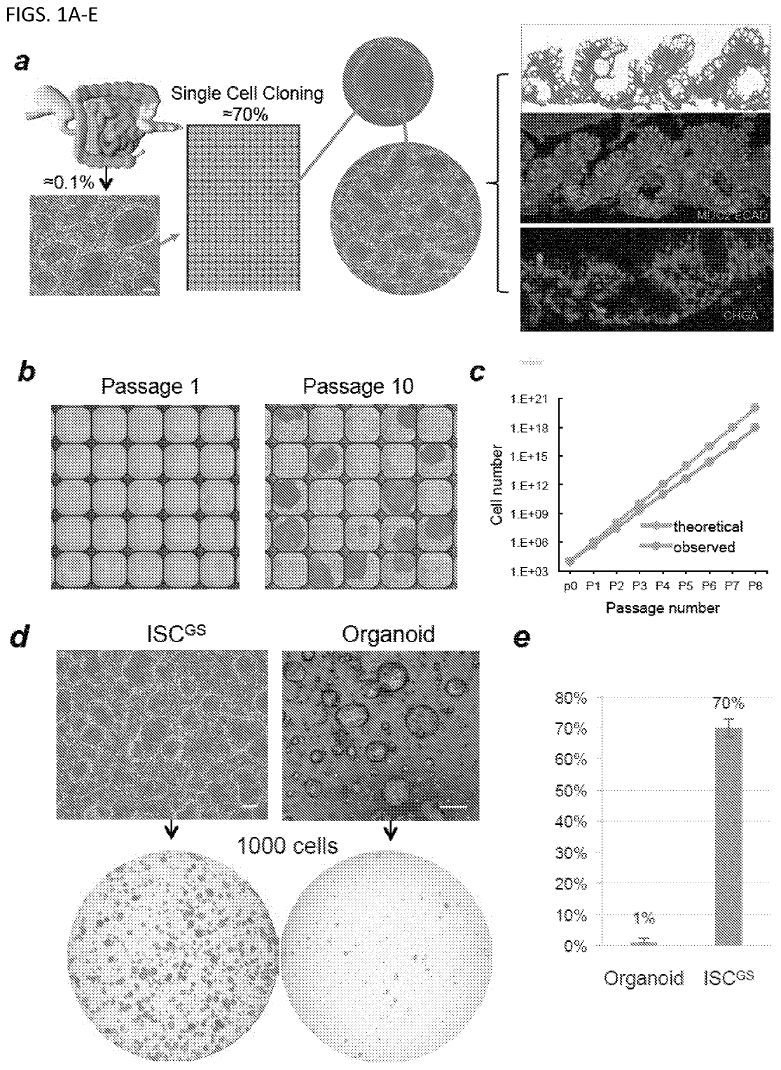

Herein the inventors compared the fate of cloned ground state ISCs in both organoid and ground state culture systems. Libraries of ground state ISCs were derived from endoscopic biopsies of human terminal ileum by seeding single cell suspensions onto lethally irradiated 3T3-J2 cells in specialized media (detailed in Methods) [13](FIG. 1A). Single colonies were sampled, dissociated to single cells, and FACS-sorted as single cells to 384-well plates that formed colonies at a rate of approximately 70% (71+/−5.3%). Single clones were selected from these 384-well plates, expanded as clonal populations, and validated by in vitro differentiation in air-liquid interface (ALI) cultures that recapitulated terminal ileum epithelia dominated by Muc2+ goblet cells (FIG. 1a). These ground state ISCs show a remarkably consistent clonogenicity (approx. 71%) across 10 passages and achieve a proliferative expansion that is both close to the theoretical limit [14] (FIG. 1b,c) and estimated to be 250-fold faster than that reported for organoid expansion [9,11]. Using these validated ground state ISC clones, the inventors seeded 2,000 single cells in parallel into ground state and organoid cultures. After two weeks, the inventors determined the clonogenicity of single cells from the ground state and organoid cultures by plating 1000 single cells onto assay plates. While the ground state ISCs maintained their 70% (73+/−4.2%) clonogenicity, cells from organoids showed a dramatic loss of clonogenicity to less than 1% (0.96+/−0.11%) (FIG. 1c,d). These data are consistent with previous reports that Lgr5+ cells represent minor fraction of organoid cells and that the clonogenicity of organoid cells is very low [9,10].

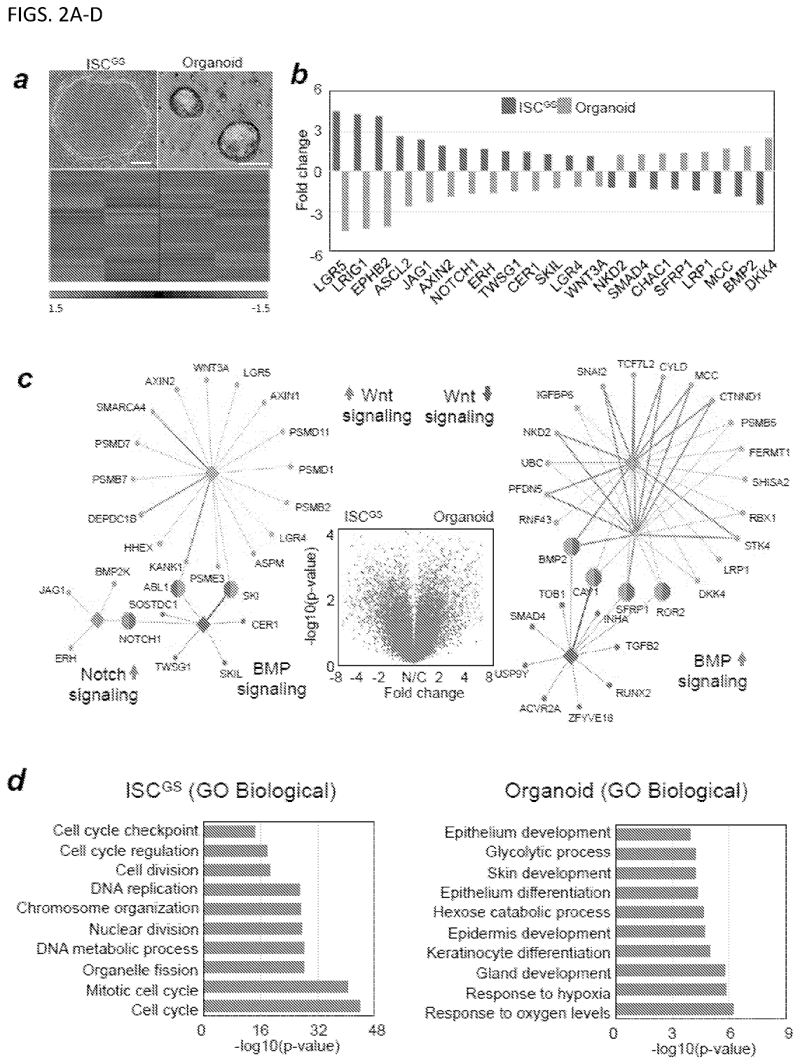

Given the profound loss of clonogenic ISCs in organoid culture, the inventors asked whether whole genome expression profiling could yield insights into the fate of cloned ISCs grown in these two systems. This comparison revealed vast differences in gene expression between ground state ISCs and those adapted to organoid growth involving some 1150 genes (Log 2 2-fold, p<0.05), including the loss in organoids of ISC markers such as Lgr5, EphB2, Lrig1, Ascl2, and Lgr4 (FIGS. 2a,b). In addition, the inventors checked for genes associated with the self-renewal pathways such as WNT, NOTCH and BMP and noted differences in their expression patterns in ISCs and organoids. [15] (FIG. 2c). In particular, positive regulators of WNT and NOTCH pathways were upregulated in ground state ISCs, and while negative regulators of these pathways dominated the organoid cells (FIG. 2c). Conversely, BMP signaling pathways were up in organoids and low in ground state ISCs (FIG. 2c), consistent with a general upregulation of BMPs with increasing differentiation along the crypt-villus axis [reviewed in 15]. Examination of the most significant, differentially expressed gene sets enrichment profile of Gene Ontology (GO) biological process showed that Proliferation and Cell Cycle Regulation dominated the ground state ISCs, while organoids where characterized by gene sets involving hypoxia, development, and differentiation (FIG. 2d).

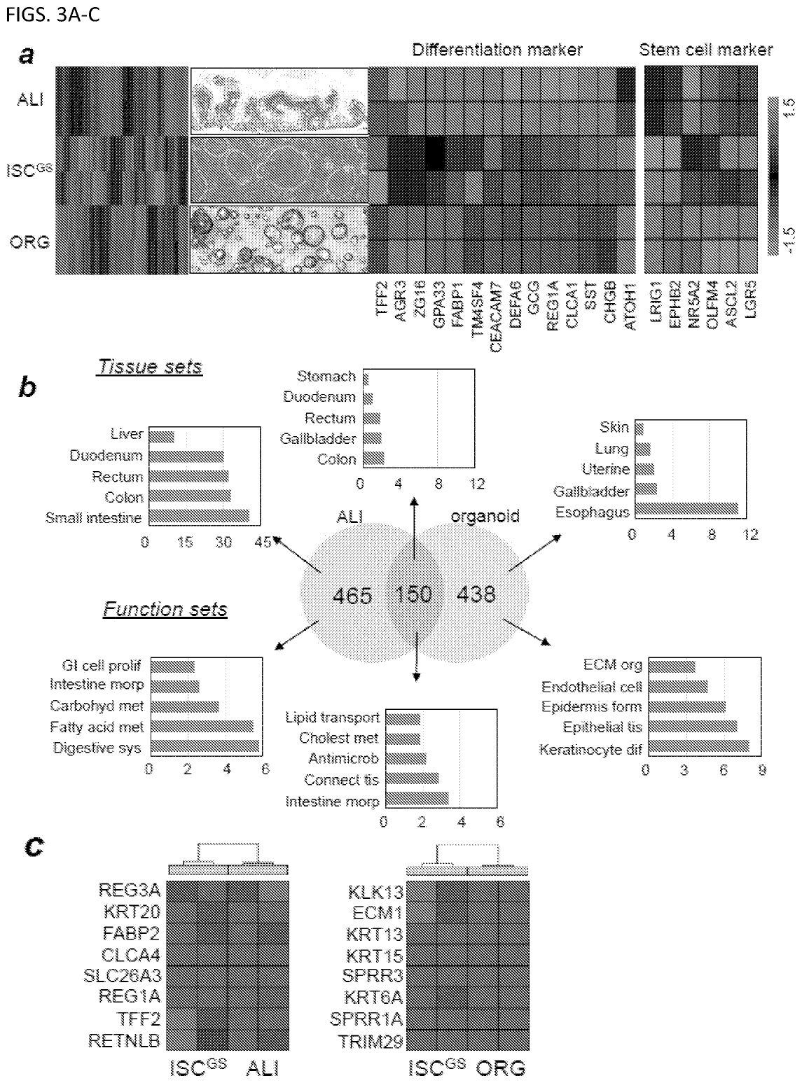

Taken together, these data suggest that the majority of organoid cells have diverged from the highly proliferative and clonogenic states that mark ground state ISCs, perhaps as a consequence of initiating programs of differentiation to cell types typical of the human terminal ileum. To assess whether organoid cells had indeed initiated differentiation programs, the inventors compared the gene expression differences when ground state ISCs were adapted to organoids versus ALI-differentiated terminal ileum epithelia generated from ground state ISCs (FIG. 3a). ALI differentiated ground state ISCs expressed a wide range of markers of goblet, Paneth, and enteroendocrine cells associated with colonic epithelia, whereas organoids derived from the same cloned ground state ISCs showed none of these colonic markers. These findings argue that colonic differentiation is not the explanation for the loss of clonogenicity and proliferation by the majority of organoid cells, and suggest the answer lies elsewhere.

To further explore the fate of ISCs in organoid culture, the inventors compared genes that were differentially represented in ALI-generated colonic epithelium with those overrepresented in organoid culture (FIG. 3b). As expected, the differentiation of ground state ISCs in ALI culture resulted in Tissue Specific gene sets denoted Small intestine, Colon, and Rectum. Unexpectedly, the high-confidence Tissue Specific gene sets induced in organoid culture identified Esophagus and Epidermis, squamous epithelia quite distinct from the gastrointestinal tract (FIG. 3b). Analysis of these expression datasets by Functional gene sets tied the ALI-generated colonic epithelia to Digestive System Development as well as Carbohydrate and Fatty Acid Metabolism, while the organoid Function gene sets were Keratinocyte Differentiation, ECM organization, and Epidermal Development. At the individual gene level, the ALldifferentiated ISCs expressed a host of established marker genes for intestinal epithelium (RETNLB, KRT20, REG3A, FABP2), while cells in the organoids expressed genes linked to squamous lineages, including TRIM29, KRT15, KRT6A, SPRR1A, and SPRR3 (FIG. 3c)

In summary, organoids are far and away the dominant reification of adult stem cells in biology and prospective regenerative medicine and yet the precise relationships between organoid cells and clonogenic stem cells remain surprisingly shadowed and unexplored. The inventors conclude from the studies here that the vast majority of organoid cells have exited any definable stem cell state based on gene expression profiling and quantitative clonogenicity assays, and cannot be rescued or “dedifferentiated” to a stem cell state by conditions that support the cloning and propagation of ground state ISCs. While the inventors had anticipated that the predominant population of “non-stem cells” in organoids would have progressed towards intestinal differentiation, the gene expression profiling did not support this expectation and in fact uncovered gene sets more commonly associated with stratified epithelia such as the esophagus and epidermis. These studies add to concerns [16] regarding the nature of cells in organoids, their relationship to ground state stem cells of regenerative tissues [11,17-20] and, in the run-up to regenerative medicine, the functional and molecular properties of cells in both systems.

### REFERENCES FOR EXAMPLE 1

The following references, to the extent that they provide exemplary procedural or other details supplementary to those set forth herein, are specifically incorporated herein by reference.
1. Evans M. Embryonic stem cells: the mouse source—vehicle for mammalian genetics and beyond Chembiochem. 9, 1690-1696. (2008).
2. Stevens L. C. The development of teratomas from intratesticular grafts of tubal mouse eggs. J Embryol Exp Morphol. 20, 329-341 (1968).
3. Siminovitch I, McCulloch E A, and Till J E. The distribution of colony-forming cells among spleen colonies. J Cell Comp Physiol. 1963 December; 62:327-36.
4. Cheng, H. and Leblonde, C P. Origin, differentiation and renewal of the four main epithelial cell types in the mouse small intestine V. Unitarian theory of the origin of the four epithelial cell types. American Journal of Anatomy 141, 537-561 (1974).
5. Barker N. et al. Identification of stem cells in small intestine and colon by marker gene Lgr5. Nature 449, 1003-1007 (2007).
6. Sangiorgi E and Capecchi M R. Bmi1 is expressed in vivo in intestinal stem cells. Nat Genet. 40, 915-920 (2008).
7. Jadhav U. Dynamic reorganization of chromatin accessibility signatures during dedifferentiation of secretory precursors into Igr5+ intestinal stem cells. Cell Stem Cell 21, 65-77 (2017)
8. Smith N. R. et al. Monoclonal antibodies reveal dynamic plasticity between Lgr5- and Bmi1-expressing intestinal cell populations. Cell Mol Gastroenterol Hepatol. 6, 79-96 (2018).
9. Sato, T. et al. Growing self-organizing mini-guts from a single intestinal stem cell: mechanism and applications. Science 340, 1190-1194 (2013).
10. Wang F. et al. Isolation and characterization of intestinal stem cells based on surface marker combinations and colony-formation assay. Gastroenterology 145, 383-395 (2013).
11. Wang X. et al. Cloning and variation of ground state intestinal stem cells. Nature 522, 173-178 (2015).
12. Yamamoto Y. et al. Mutational Spectrum of Barrett's Stem Cells Suggests Paths to Initiation and Progression of a Precancerous Lesion. Nat Commun. 7, 1-10 (2016).
13. Duleba M. et al. An Efficient Method for Cloning Gastrointestinal Stem Cells from Patients via Endoscopic Biopsies. Gastroenterology 156, 20-23 (2019).
14. Reynolds B A and Rietze R L. Neural stem cells and neurospheres—re-evaluating the relationship. Nature Methods 2, 333-336 (2005).
15. Date S and Sato T. Mini-gut organoids: reconstitution of the stem cell niche. Annu Rev Cell Dev Biol. 31, 269-289 (2015).
16. Simian M and Bissell M J. Organoids: A historical perspective of thinking in three dimensions. J Cell Biol. 216, 31-40 (2017).
17. Barrandon Y and Green H. Three clonal types of keratinocyte with different capacities for multiplication. Proc Natl Acad Sci USA. 84, 2302-2306 (1987).
18. Green H. The birth of therapy with cultured cells. Bioessays 30, 897-903 (2008).
19. Rama P. et al. Limbal stem-cell therapy and long-term corneal regeneration. N Engl J Med. 363, 147-155 (2010).
20. Hirsch T. et al., Regeneration of the entire human epidermis using transgenic stem cells. Nature 551, 327-332 (2017).

### Example 2: Amplification of Proinflammatory Stem Cell Variants in Crohn's Disease

Crohn's disease (CD) is a progressive inflammatory and fibrotic disorder of the gastrointestinal tract tied to a cycle of aberrant interactions between immune cells, intestinal microbes, and intervening intestinal barriers, though the primary defects remain unclear. Here we perform novel clonogenic analyses of stem cells derived from intestines of patients with CD. We show that CD stem cell libraries are dominated by two variants that display incessant pro-inflammatory and pro-fibrotic signaling. Transplantation of these variants to immunodeficient mice triggers key features of CD including leukocyte infiltration and fibrosis. These variants, which exist at low levels in control and fetal intestines, display anomalous gastric fates that likely dictate their inflammatory and fibrotic properties. Together, this work links CD to the amplification of minor stem cell variants whose conventional roles are directed at ancient pathogens, and suggests mechanistic analogies to chronic obstructive pulmonary disease (COPD) and perhaps other inflammatory conditions.

INTRODUCTION: Crohn's disease (CD) is an inflammatory bowel disorder marked by transmural lesions that frequently progress to strictures, fistulas, or perforations requiring surgical intervention^{1,2}. Though immunosuppressants and anti-inflammatory biologics can slow the progression of CD, it is not clear that they have lessened the need of surgical intervention, an impasse that has fueled the search for therapeutic targets more proximal to the disease^{2,3}. This search is complicated by the large environmental contribution to this disease reflected by the low concordance among monozygotic twins⁴, and by the polygenic nature of the remaining, inherited risk. Nevertheless, genome-wide association studies (GWAS), coupled with intestinal pathophysiology studies are beginning to define the underlying biological and genetic structure of CD^{5,6}. In particular, these studies reveal a stunning overlap of risk loci for CD and mycobacterial infections and implicate genes of adaptive and innate immune processes in the containment of gut microbes⁷⁻¹². The restricted expression of multiple genes highlighted in GWAS studies in intestinal mucosa support an emerging “barrier defect” hypothesis involving deficiencies in anti-microbial functions of Paneth cells in CD patients¹³, defective autophagy processing of microbial antigens by mucosal epithelial cells^{7,8,13-18}, and altered responsiveness of mucosal immune cells¹⁹. Despite compelling data on barrier abnormalities in CD, it remains unclear whether these mucosal defects are primary events or secondary consequences of the inflammatory state of this disease. It is also unclear how defective barrier function might explain the alternate regional presentations of Crohn's^{20,21}, its “skip-lesion” patterning, or the high rates of disease recurrence following ileocolonic resection^{2,22}.

The present work sought to address barrier properties in CD through a comparative clonal analysis of intestinal stem cells derived from endoscopic biopsies of CD patients and controls. Unlike libraries of clonogenic cells from control cases, which are dominated by normal terminal ileum stem cells, we show that those of CD cases are beset by discrete variants marked by constitutive inflammatory signatures including genes associated with inflammatory bowel disease and with the response to multiple human pathogens. Consistently, xenografts of these variant cells in highly immunodeficient mice drive host inflammatory and fibrotic responses reminiscent of those seen in CD. The properties of these variant stem cells suggest that a component of CD is the overrepresentation of intrinsic “sentinel” cells that normally function to protect the gastrointestinal tract from ancient human pathogens.

Results

Clonogenic Analysis of CD Stem Cells

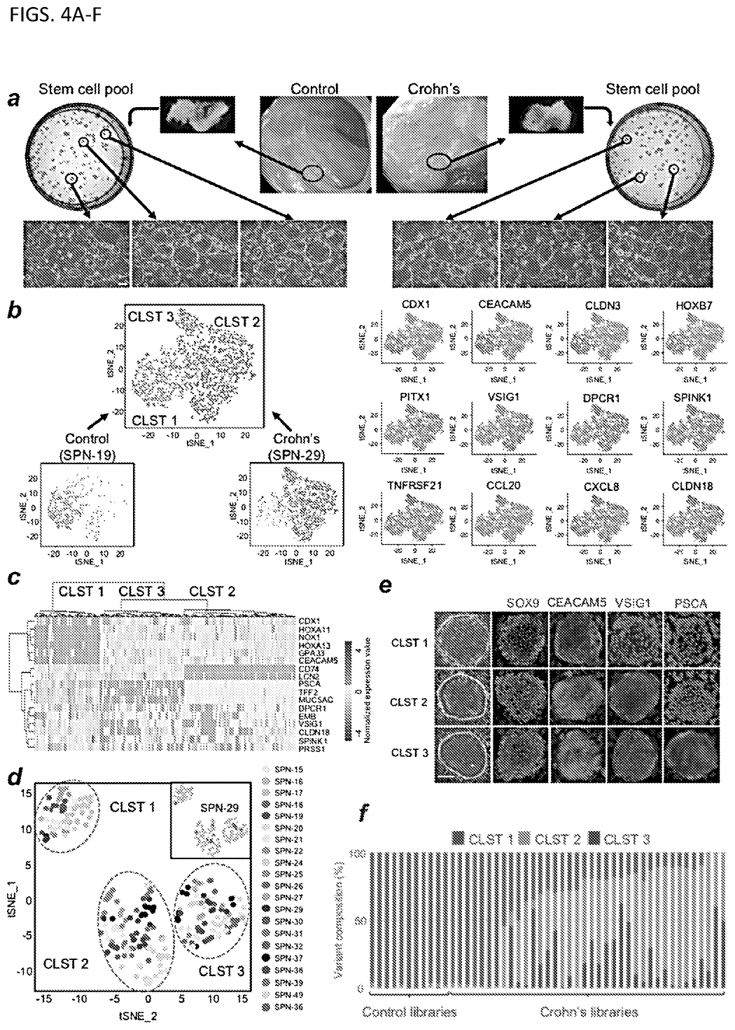

We generated mucosal stem cell ‘libraries” of 100-300 clones from 1 mm endoscopic biopsies taken from the terminal ileum of patients with and without CD using the processes described here, which enables the isolation of single cell-derived lines^{23,24} (FIG. 4a). Analysis of the whole stem cell libraries from control cases by single cell RNA sequencing (scRNAseq) yielded tSNE profiles that were dominated by a single cluster, whereas those CD patients showed three clusters including the one that predominates in controls (Cluster 1, or CLST1) and two more abundant, clusters (CLST2 and CLST3) (FIG. 4b).

To probe this apparent stem cell heterogeneity evident among terminal ileum stem cells of CD patients, we randomly sampled 275 clones from libraries from 21 patients. Each of these clones was expanded and assessed by RT-PCR for expression of 16 marker genes that distinguish CLST 1, 2, and 3 in whole genome expression (fold change >1.5; p<0.05) profiles (FIG. 4c). A tSNE analysis of these data showed that the 275 clones fell into three groups consistent with CLST1, 2, and 3 stem cells (FIG. 4d). For multiple CD patients, including SPN-29, this random sampling yielded patient-matched representative clones for CLST 1, 2, and 3 (FIG. 4d, inset). To quantify the relative proportions of CLST1, 2, and 3 clones across stem cell libraries generated from control and CD cases, we leveraged three markers (CEACAM5 for CLST1; VSIG1 for CLST2+3; and PSCA for CLST3) for quantitative fluorescence-activated cell sorting (FACS) (FIG. 4e,f). Among stem cell libraries representing CD cases, the variant clones (CLST2+3) made up approximately 64.6+/−32.6% of all clones, compared to 1.2+/−1.4% in control cases.

CD Stem Cells Committed to Upper GI Fate

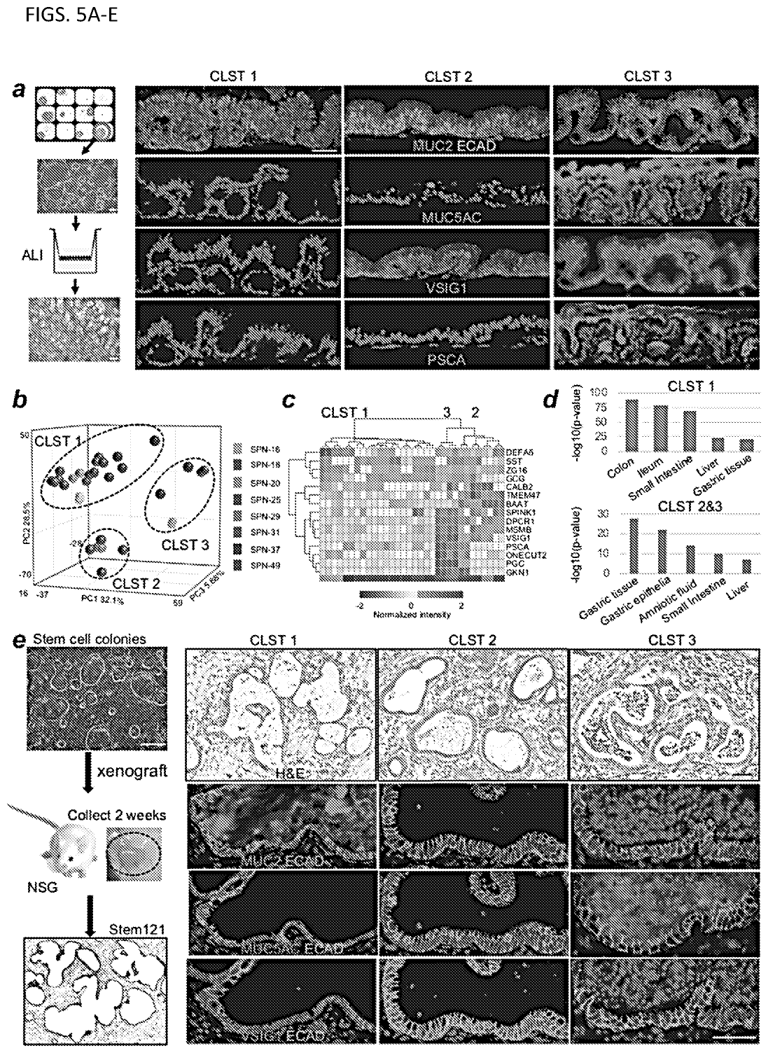

To further characterize the stem cell variants identified in normal and CD libraries, we examined their fate commitment upon differentiation in air-liquid interface (ALI) cultures^{23,24}(FIG. 5a). CLST1 stem cells, the predominant stem cell in control libraries, differentiated to MUC2-expressing goblet cells, CHGA-expressing enteroendocrine cells, and DEFA6-expressing Paneth cells typical of terminal ileum mucosa (FIG. 5a). In contrast, CLST2 and 3 stem cell clones differentiated to epithelia devoid of obvious goblet cells, enteroendocrine cells, or Paneth cells, and instead expressed genes such as MUC5AC, VSIG1, and PSCA more typical of gastric epithelia (FIG. 5a). Whole genome expression analysis of differentiated epithelia of multiple CLST1, 2, and 3 clones (fold change >1.5; p<0.05) showed that they were respectively committed to distinct epithelia (FIG. 5b,c). This analysis showed markers of colonic epithelia in differentiated CLST1 clones, whereas CLST2 and CLST3 clones formed epithelia with markers of proximal gastrointestinal tract (FIG. 5c). At the whole genome expression level the differentiated CLST1A clones were similar to ileocolonic epithelia while the CLST2 and CLST3 clones differentiated to gastric-like epithelia. cells A comparison of the whole genome expression profiles of these in vitro differentiated epithelia with published epithelial profiles showed that CLST1 epithelia were most similar to colon and ileum while CLST2 and CLST3 were most closely related to gastric epithelia (FIG. 5d).

We also assessed the fate commitment of the CLST1, 2, and 3 clones in vivo following subcutaneous transplantation to immunodeficient (NODscid IL2ra^{null} [NSG] mice²⁵ (FIG. 5e). Xenografts of each clone type formed polarized epithelia within several days that were marked by the human-specific antibody STEM121²⁶. Clonally-derived CLST1 epithelia were further marked by MUC2 expression, while CLST2 and CLST3 epithelia displayed MUC5AC, VSIG1, and CLDN18 expression (FIG. 5e). These in vivo data support the notion that the dominant clone type in control cases is committed to a terminal ileum fate while those predominating in CD mucosa have a gastric-like fate.

CD Stem Cells Drive Inflammation

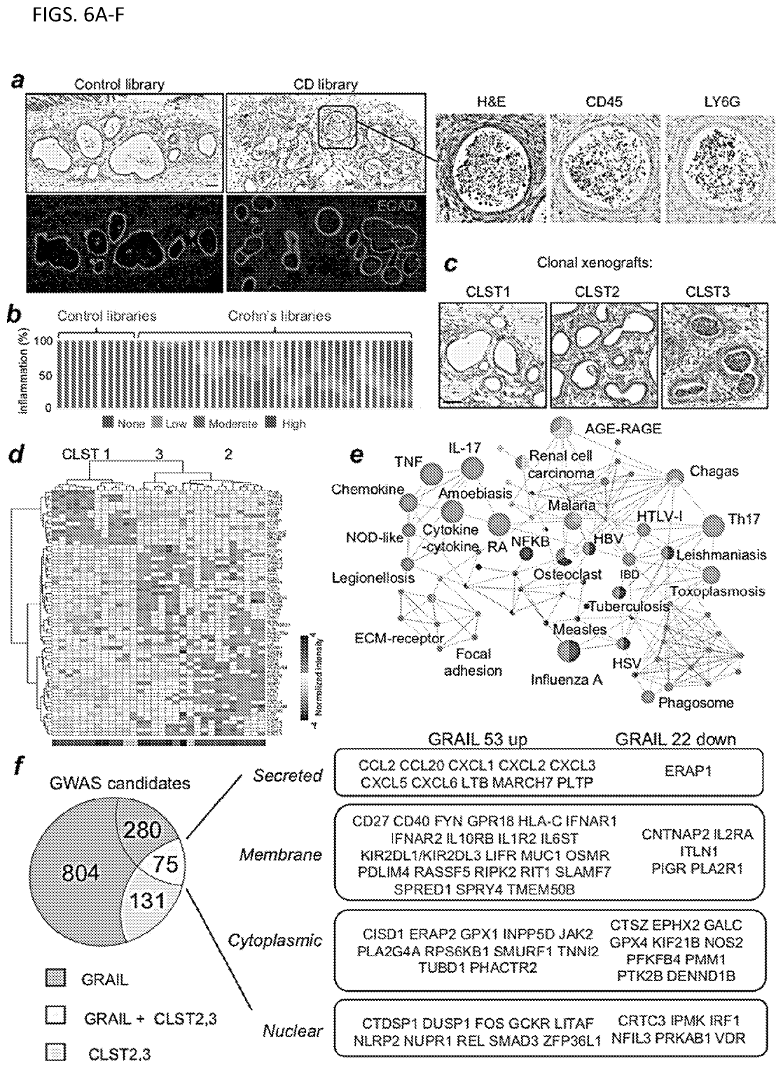

Xenografts derived of Crohn's stem cell libraries showed higher extra- and intra-luminal cellularization compared with control libraries (FIG. 6a). The intraluminal cells in the Crohn's library xenografts appeared to be leukocytes by hematoxylin-eosin (H&E) staining, a notion we confirmed by antibodies to the hematopoietic lineage marker CD45 and the neutrophil marker LY6G (FIG. 6a, outset). We quantified the extent of neutrophil accumulation²⁷ in these cysts across the 11 control and 38 Crohn's library xenografts using morphometric standards, which showed that this inflammatory host response was a major distinguishing feature of Crohn's library xenografts (FIG. 6b). The extent of neutrophilic inflammation in these library xenografts revealed a strong correlation (R=0.82) with the fractional representation of CLST3 clones. Consistently, xenografts of individual CLST1, 2, and 3 clones showed that only CLST3 xenografts infiltration by neutrophils in the NSG immunodeficient mice (FIG. 6c).

Given the strong host neutrophil response to Crohn's stem cell libraries, and CLST3 clones in particular, we asked if these stem cell variants showed differential expression of inflammatory genes. Gene set enrichment analysis showed that CLST1 stem cells were dominated by metabolic gene sets, including Bile Acid Transport and Zinc Homeostasis. In contrast, the top 30 pathways of differentially expressed genes for both CLST2 and CLST3 were related to inflammatory and immune responses, including Ebola Virus Responses, Allograft Rejection, Complement Cascades, and Lung Fibrosis for CLST2, and EGFR signaling, Oncostatin M, TGF-beta, and Hepatitis C and Hepatitis B Responses for CLST3 clones. An expression heatmap of curated genes linked to inflammatory bowel disease showed that normal terminal ileum stem cells (CLST1) expressed high NOX1, GUCY2C, RETNLB, CD200, TLR4, and ITLN1 whereas CLST2 and CLST3 expressed an array of chemokines (e.g. CXCL1-5), receptors and ligands for interleukins 1, 6, 8, 12, and 18 pathways, the Oncostatin M pathway²⁸ (OSMR, IL6ST, LIFR), interferon signaling²⁹ (e.g. INFAR1, INFAR2, IL10RB, INFGR1), and angiogenesis (VEGFA, HIF1A) (FIG. 6d). Network analysis of the differentially expressed inflammatory genes for CLST2 and CLST3 revealed multiple interacting nodes including IL-17, Th17, TNF, Chemokines, Cytokines, Inflammatory Bowel Disease, immune responses to viral infection (Influenza A, Measles, HTLV-1, HSV, HBV, and an array of bacterial (e.g. Legionellosis, Tuberculosis) and intracellular protozoan (e.g. Malaria, Toxoplasmosis, Leishmaniasis, and Chagas) diseases^{30,31} (FIG. 6e).

Genome-wide association studies of Crohn's patients have been enormously successful in identifying risk loci and candidate genes that have shaped our understanding of this disease^{1,7-12}. As the CLST2 and CLST3 stem cells derived from Crohn's terminal ileum showed pro-inflammatory gene profiles and corresponding activities host responses upon transplantation to mice, we asked if the differentially expressed genes in CLST2 and CLST3 clones include genes implicated by GWAS in the risk for Crohn's. Using gene expression criteria (1.5-fold, p<0.05), we identified 75 genes differentially expressed in CLST2 and CLST3 stem cells previously implicated in Crohn's by three distinct GWAS analyses^{7,8,12} and by computational analyses of linkage disequilibrium (LD) blocks (e.g. GRAIL³²; FIG. 6f). A majority of the matches we identified involve single genes among several within LD blocks (e.g. NUPR1 at rs26526), and several highlight multiple genes coordinately upregulated within a single DL block containing related genes such as CXCL1, 2, 3, and 5 at rs2472649¹¹.

CD Stem Cells Trigger Fibrotic Responses

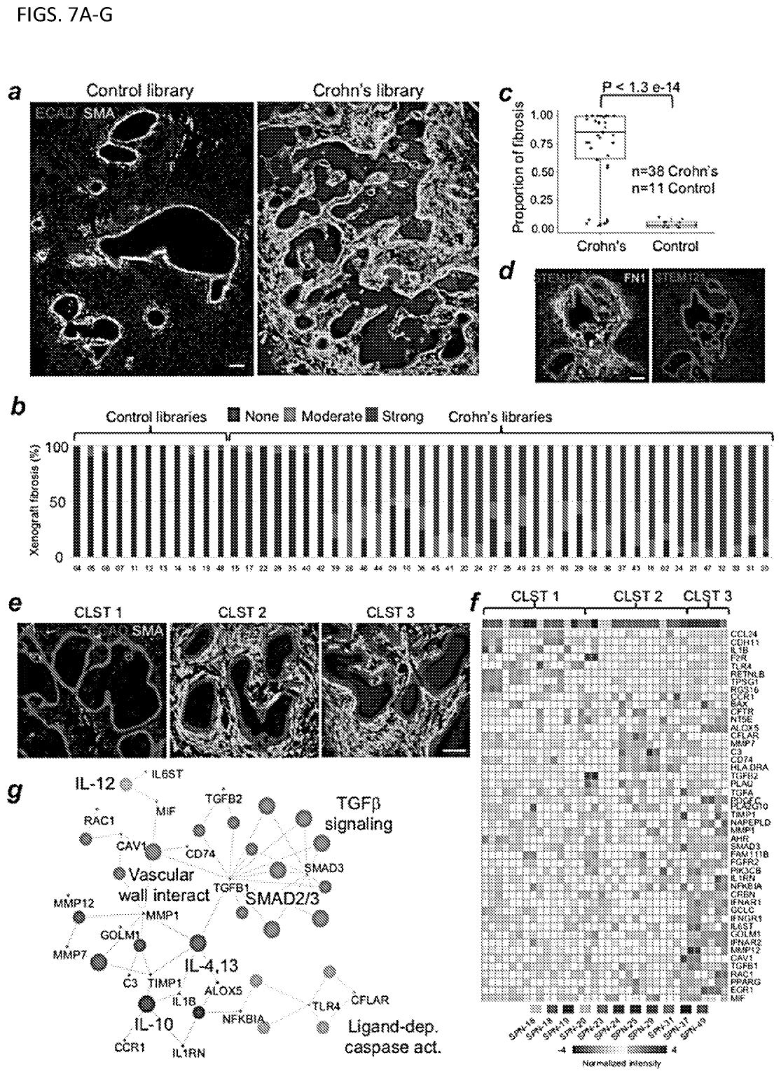

Fibrosis is a major complication of CD and a risk factor for bowel obstruction requiring surgical intervention^{2,33,34}. Alpha-smooth muscle actin (a-SMA)-expressing myofibroblasts have been linked to the fibrosis in CD and other chronic inflammatory conditions^{35,36}. We noted abundant submucosal cells in xenografts of CD stem cell libraries that were not evident in xenografts of control stem cell libraries (c.f. FIG. 6a), and asked if these submucosal cells included myofibroblasts. α-SMA immunofluorescence showed these submucosal cells to be largely comprised of myofibroblasts across all xenografts derived from CD stem cell libraries (FIG. 7a). Quantification of submucosal regions occupied by myofibroblast networks further underscored the extent of this host response to epithelia generated from CD libraries versus libraries from control patients (FIG. 7b,c). The extent of submucosal myofibroblast accumulation showed a strong correlation (R=0.96, p<2.2e-16) with the respective VSIG1+ fraction of clones within the CD libraries determined by FACS profiling. We note here that the myofibroblasts in these xenografts, which express both a-SMA and fibronectin (FN1), do not react to anti-human STEM-121 antibodies (FIG. 7d) and therefore are of host origin.

To dissect which, if any, of the three clone types could unilaterally promote fibrotic responses similar to those seen in xenografts of unfractionated CD libraries, we performed xenografts with discrete clones of CLST1, CLST2, and CLST3. Analysis of these clonally-derived xenografts showed that both CLST2 and CLST3 clones evoked a strong and uniformed recruitment of submucosal myofibroblasts marked by α-SMA immunoreactivity (FIG. 7e). Consistent with the pro-fibrotic activity of CLST2 and CLST3 clones, both CLST2 and CLST3 clones expressed multiple genes in TGF-β pathways linked to fibrosis in chronic diseases³¹ (FIG. 7f,g).

CD Stem Cells in Fetal Terminal Ileum

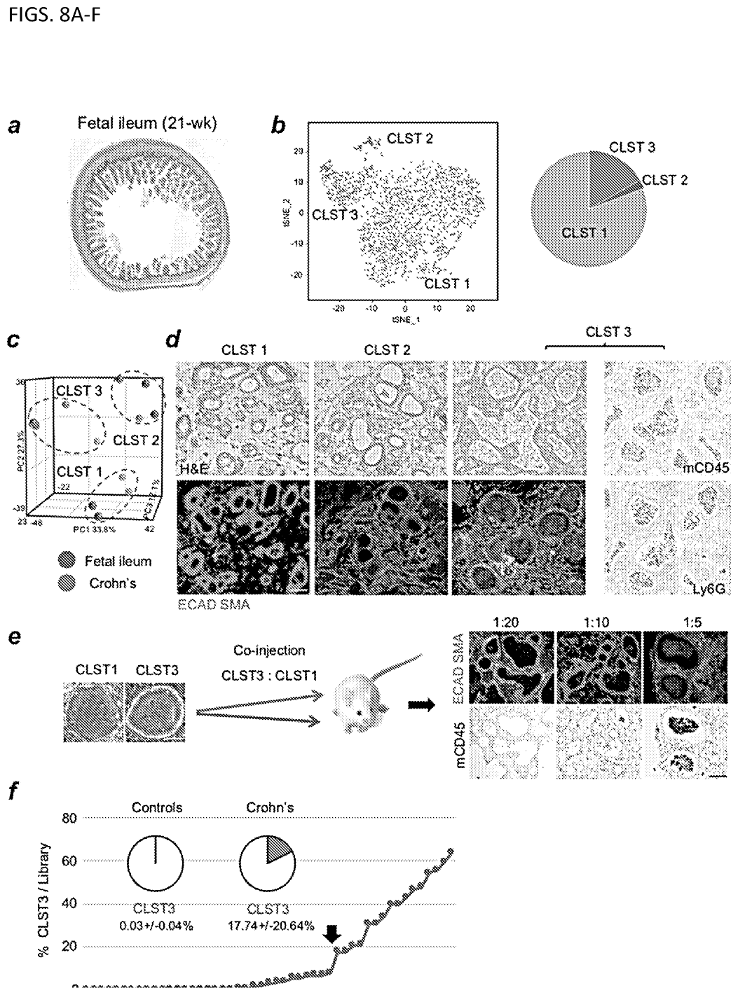

Determining the origins of the variant stem cells in CD terminal ileum will be important to understand their roles, if any, in this disease. Clone libraries generated from control cases in this study also displayed CLST2 and CLST3 variants seen to dominate CD cases, albeit at very minor ratios to the normal, CLST1 stem cells (c.f. FIG. 4 b,d,e). However, it was conceivable that some of the control cases used in this study harbored subclinical disease and that the minor populations of CLST2 and CLST3 clones in these cases were reflections of early disease. We therefore asked if CLST2 and CLST3 clones were present in clone libraries generated from prenatal terminal ilea of 21- and 22-week fetal demise cases (FIG. 8a). tSNE profiles of scRNAseq data of these libraries showed that while these fetal libraries were dominated by CLST1 cells, we could also detect cells with gene expression profiles consistent with those of CLST2 and CLST3 clones in Crohn's. By sampling single cell-derived clones in 384-well plates, we succeeded in cloning representatives of each of the three stem cell types seen in CD (SPN-29) based on both whole genome expression profiles and RT-PCR analyses of marker gene transcripts (FIG. 8b,c). Importantly, the xenografts of these fetal clones behaved in a manner indistinguishable from their corresponding clone types from CD biopsies, with CLST2 clones yielding submucosal fibrosis, CLST3 both fibrosis and neutrophilic inflammation, and CLST1 clones triggering neither of these host responses (FIG. 8d).

The preexistence of CLST2 and 3 clones in fetal and control terminal ilea suggested that low ratios of these cells could be maintained without provoking fibrosis or inflammation and yet at higher levels could promote pathology. To test this notion, we examined the properties of xenografts generated from co-transplantations of CLST1 and CLST3 cells at precise ratios (FIG. 8e). Histology and immunofluorescence analyses of xenografts consisting of 5% CLST3 stem cells showed no obvious phenotype, 10% CLST3 showed marginal but detectable inflammation and fibrosis, while those having 20% or more CLST3 cells showed abundant inflammation and fibrosis (FIG. 8f). Thus, the inflammatory inflection point in this model system appears to be centered around 10-20% of CLST3 stem cells in the overall clone population, a range that includes the median percentage of CLST3 cells in libraries of clonogenic cells of patients with CD (between 8-18%; FIG. 8f,g).

CD Variants Mirror Gastric Inflammation

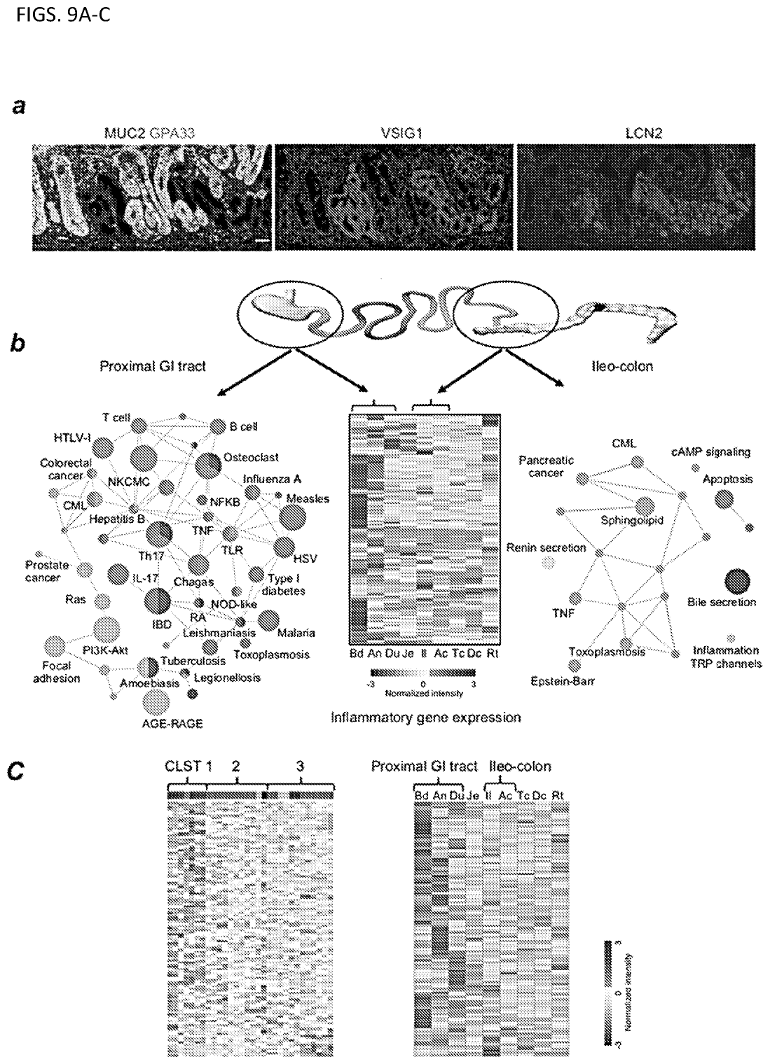

The CLST2 and CLST3 variants displayed gene expression profiles more similar to gastric stem cells than those of terminal ileum mucosa from which they were derived (c.f. FIG. 5a,c,d). Reports of “pyloric” or “gastric” metaplasia in association with mucosal ulcerations in CD date back nearly 70 years³⁸⁻⁴⁰. We examined histological sections of terminal ileum of patients with CD using antibodies to markers of CLST1, CLST2, and CLST3 cells. Consistent with the co-existence of the normal and variant cells in CD stem cell libraries, we identified regions of normal (GPA33+/MUC2+) mucosa similar to those produced by CLST1 clones intermingled with regions of gastric metaplasia marked by CLST2 and CLST3 markers (e.g. VSIG1 with or without LCN2) in the CD biopsies from multiple patients (FIG. 9a). This apparent link between pro-inflammatory and pro-fibrotic CLST2 and CLST3 cells and reports of gastric metaplasia at CD lesions is at odds of the “reparative” functions proposed for these gastric metaplasia³¹. To address this disparity, we exploited our ability to clone stem cells from all segments of the human gastrointestinal tract (Wang et al., submitted) to probe the properties of gastric stem cells in detail. Our findings with stem cells from across the gastrointestinal tract showed these cells are epigenetically committed to regional fates and display regionally appropriate metabolic functions such as nutrient processing and bile acid recycling (Wang et al., submitted). Aside from these lineage and metabolic specificities, stem cells of the proximal gastrointestinal tract, including those of the gastric body, antrum, and duodenum, showed a broad differential expression of pro-inflammatory and pro-fibrotic genes relative to more distal regions of the gastrointestinal tract (FIG. 9c). A network analysis of the pro-inflammatory and pro-fibrotic genes expressed by normal proximal gastrointestinal tract stem cells revealed many of the same nodes seen in CLST2 and CLST3 cells, including IL-17, Th17, TNF, Inflammatory Bowel Disease, as well as multiple nodes related to the response to viral (HSV, HTLV-1, Influenza A), bacterial (Legionellosis, Tuberculosis), and protozoan (Chagas, Amoebiasis) pathogens (FIG. 9b). The overall similarity of the inflammatory gene networks in the CD variant stem cells and those of the proximal gastrointestinal tract is also reflected by at the level of individual inflammatory genes across these same cells (FIG. 9c). These data link the CLST2 and CLST3 variants in CD to pre-existing stem cells with lineage and functional properties of those of the upper gastrointestinal tract which may themselves function in the response to pathogen incursions.

### DISCUSSION

CD remains an untamed condition with serious, life-long complications despite the advent of advanced therapeutics^{1,2}. Although the product of contributions from the immune system, intestinal microbiome, and mucosal barriers^{5,18,19,42,43}, the present study suggests that variant intestinal stem cells may play an unexpectedly active role in this condition and could figure in therapeutic strategies.

The CLST2 and CLST3 variants that numerically dominate the stem cell repertoire of CD display three features that could render them pathogenic for the disease. For one, their differentiation to epithelia devoid of goblet and Paneth cells could directly affect the barrier to intestinal microbes and trigger an inflammatory response by immune cells^{7,8,13-6,18}. However, both the CLST2 and CLST3 cells show a broad and constitutive expression of inflammatory genes that sum to a concatenated network of signaling pathways involving IL-1, IL-6, IL-12, IL-17, IL-18, and oncostatin M, among others, all of which have been previously linked to IBD^{1,28,44}. The notion of pathogenicity of CLST2 and CLST3 stem cells was extended further by an analysis of host responses to xenografts of these clones in immunodeficient mice. CLST3 clones, but not to those of CLST2 or CLST1, induced a strong trans-epithelial infiltration by host neutrophils. This neutrophil response requires multiple steps including leukocyte rolling at local vasculature, extravasation across the endothelia, chemotaxis to the xenograft, and finally trans-epithelial migration^{27,45}, all processes conceivably supported by constitutive signaling pathways operating in CLST3 clones. Given that the NSG host strain is profoundly deficient in hematopoietic lineages including those of B and T cell, natural killer cells, and monocyte-derived macrophages and dendritic cells²⁵, the range and extent of host immune responses to CLST3, and certainly CLST2 stem cell, are probably underestimated in these trials. Additional strains that spare larger segments of the immune cell repertoire could extend our understanding of the inflammatory consequences of CLST2 and 3, as would syngeneic models involving hypothetical analogs of CLST2 and CLST3 in genetically tractable organisms.

Fibrosis remains one of the most insidious features of Crohn's and represents a major risk for stenosis and surgical intervention²⁵. The CLST2 and CLST3 stem cell variants constitutively express pro-fibrotic gene sets and, upon xenografting to mice, drive extensive recruitment of host-derived myofibroblasts. Given the highly immunodeficient nature of the murine host, the robust induction of myofibroblasts by CLST2 and CLST3 xenografts must be occurring in the absence T or B cells, NK cells, and monocyte-dependent lineages. These finding argue that the CLST2 and CLST3 variants are sufficient for unilaterally initiating a strong fibrotic response that, in Crohn's, might be augmented by the actions of cells of the adaptive and innate immune systems^{42,46}.

The source of the pathogenic variants identified here will be relevant to understanding the origins and cyclical nature of Crohn's disease, its high rates of post-surgical recurrence, and strategies for its mitigation. Variants with similar gene expression profiles and pro-inflammatory and pro-fibrotic activities as xenografts were identified in control pediatric and adult terminal ilea biopsies, albeit at low ratios to the CLST1 stem cells. While these studies argue for a pre-existence of these putative pathogenic cell types, they provide no hints as to why these variant cell populations expand in the first place, though genetics and adverse gastrointestinal events may selectively favor such expansions. We also note that the majority of patients analyzed in this study had achieved a clinically stable state of remission such that frank inflammation was not evident by white light endoscopy. Nevertheless, the stem cell libraries from these patients showed comparable fractions of the CLST2 and CLST3 variants as those from patients with active inflammation. This finding indicates that the broad immunosuppression and anti-inflammatory therapies employed in Crohn's, perhaps in concert with immunoregulatory T cell populations⁴⁸, override the pathogenic impact of these variants. However, the latent presence of these variants might render patients susceptible to flares as well as recurrent disease following surgical resections, and conceivably underlie the inexorable progression seen in many patients despite standard-of-care therapies.

Perhaps the most consequential feature of the similarity between Crohn's stem cells and those of gastric epithelia is that it includes the inflammatory and pro-fibrotic gene signatures defined for CLST2 and CLST3. In particular, the inflammatory signature of both Crohn's and gastric stem cells are centered about multiple pathways linked to inflammatory bowel disease, and are heavily weighted towards the response to ancient infections including tuberculosis, leishmaniasis, and toxoplasmosis first suggested by GWAS studies^{11,30,49}. These findings raise the possibility that the Crohn's disease is the consequence of the numerical expansion of variant mucosal stem cells whose intrinsic function, like those in the stomach, is to prevent incursions by ancient pathogens. If true, Crohn's disease could be mitigated by therapeutics that selectively target these variant stem cells. Lastly, the parallels between the role proposed here for minor mucosal stem cell variants in the emergence of Crohn's and that of lung stem cell variants in COPD⁵⁰ suggest that analogous scenarios might explain other chronic inflammatory diseases that impact the human condition.

Methods

In Vitro Culture of Human Terminal Ileum and Colonic Epithelial Stem Cells

Terminal ileum endoscopic biopsies were obtained from pediatric and adult Crohn's patients and functional controls lacking mucosal inflammation under informed parental consent and institutional review board approval at the Connecticut Children's Medical Center, Hartford, Conn. USA, the University of North Carolina, Chapel Hill, Chapel Hill, N.C., USA and Baylor College of Medicine, Houston, Tex., USA. Mucosal stem cell libraries^{23,24,51} were generated from 1 mm endoscopic biopsies which were collected into cold F12 media (Gibco, USA) with 5% fetal bovine serum (Hyclone, USA), and then were minced by sterile scalpel into 0.2-0.5 mm³ fragments. The minced tissue was digested in 2 mg/ml collagenase type IV (Gibco, USA) at 37° C. for 30-60 min with agitation. Dissociated cells were passed through a 70 μm Nylon mesh (Falcon, USA) to remove masses and then were washed four times in cold F12 media, and seeded onto a feeder layer of lethally irradiated 3T3-J2 cells in c-FAD media containing 125 ng/mL R-Spondin1 (R&D systems, USA), 1 μM Jagged-1 (AnaSpec Inc, USA), 100 ng/ml Human Noggin (Peprotech, USA), 2.5 μM Rock-inhibitor (Calbiochem, USA), 2 μM SB431542 (Cayman chemical, USA), 10 mM nicotinamide (Sigma-Aldrich, USA). Cells were cultured at 37° C. in a 7.5% CO₂ incubator. The culture media was changed every two days. Colonies were digested by 0.25% trypsin-EDTA solution (Gibco, USA) for 5-8 min and passaged every 7 to 10 days. Colonies were trypsinized by TrypLE Express solution (Gibco, USA) for 8-15 min at 37° C. and cell suspensions were passed through 30 μm filters (Miltenyi Biotec, Germany). Approximately 20,000 epithelial cells were seeded to each well of 6-well plate. Cloning cylinder (Pyrex, USA) and high vacuum grease (Dow Corning, USA) were used to select single colonies for pedigrees. Gene expression analyses were performed on cells derived from passage 4-10 (P4-P10) cultures.

Histology and Immunostaining

Histology, hematoxylin and eosin (H&E) staining, immunohistochemistry, and immunofluorescence were performed using standard techniques. For immunofluorescence and immunohistochemistry, 4% paraformaldehyde-fixed, paraffin embedded tissue slides were subjected to antigen retrieval in citrate buffer (pH 6.0, Sigma-Aldrich, USA) at 120° C. for 20 min, and a blocking procedure was performed with 5% bovine serum albumin (BSA, Sigma-Aldrich, USA) and 0.05% Triton X-100 (Sigma-Aldrich, USA) in DPBS(−) (Gibco, USA) at room temperature for 1 hr. The sources of primary antibodies used in this study, including anti-mucin 2 (sc-515032; Santa Cruz Biotechnology, USA), —Ki67 (550609; BD Biosciences, USA), —chromogranin A (ab15160; Abcam, UK), —alpha defensin 6 (HPA019462; Sigma-Aldrich, USA), —E-cadherin (AF648; R&D Systems, USA), —GPA33 (ab108938; Abcam, UK), —Ceacam5 (MAB41281; Novus Biologicals, USA), —PSCA (sc-80654; Santa-Cruz Biotechnology, USA), —Lipocalin2 (ab41105; Abcam, UK), —VSIG1 (HPA036310; Sigma-Aldrich, USA), —CLDN18 (HPA018446; Sigma-Aldrich, USA), —MUC5AC (ab78660; Abcam, UK), —SOX9 (ab185966; Abcam, UK), —CCL20 (MA523843; Thermofisher Scientific, USA), —TNFRSF1A (HPA004102; Sigma-Aldrich, USA), —Alpha Smooth Muscle Actin (ab7817; Abcam, UK), —Fibronectin1 (ab2413; Abcam, UK), —STEM121 (Y40410; Clonetech Laboratories, USA), —CD45 (14-0451-85; Thermo Fisher, USA) and -LY6G (MAB1037; R&D systems, USA) are listed (table S3). Secondary antibodies used here are Alexa Fluor-488 or Alexa Fluor-594 Donkey anti-goat/mouse/rabbit IgG antibody (Thermo Fisher, USA). All images were captured by using the Inverted Eclipse Ti-Series (Nikon, Japan) microscope with Lumencor SOLA light engine and Andor Technology Clara Interline CCD camera and NIS-Elements Advanced Research v.4.13 software (Nikon, Japan) or LSM 780 confocal microscope (Carl Zeiss, Germany) with LSM software. Bright field cell culture images were obtained on an Eclipse TS100 microscope (Nikon, Japan) with Digital Sight DSFi1camera (Nikon, Japan) and NIS-Elements F3.0 software (Nikon, Japan).

Stem Cell Differentiation

Air-liquid interface (ALI) culture of terminal ileum epithelial cells was performed as described^{23,24} Briefly, Transwell inserts (Corning Incorporated, USA) were coated with 20% Matrigel (BD biosciences, USA) and incubated at 37° C. for 30 min to polymerize. 200,000 irradiated 3T3-J2 cells were seeded to each Transwell insert and incubated at 37° C., 7.5% CO2 incubator overnight. QuadroMACS Starting Kit (LS) (Miltenyi Biotec, Germany) was used to purify the stem cells by removal of feeder cells. 200,000-300,000 stem cells were seeded into each Transwell insert and cultured with stem cell media. At confluency (3-7 days), the apical media on the inserts was removed through careful pipetting and the cultures were continued in differentiation media (stem cell media without nicotinamide) for an additional 6-12 days prior to harvesting. The differentiation media was changed every one or two days.

Xenografts in Immunodeficient Mice

Two to three million epithelial cells were harvested by trypsinization, mixed with 50% Matrigel (Becton Dickinson, Palo Alto) to a volume of 100 ul and injected subcutaneously in NSG (NODscid IL2ranull)²⁵ mice (Jackson Laboratories, Bar Harbor) and harvested one or two weeks later.

Flow Cytometry Analysis

Clonogenic cell libraries from patients with or without Crohn's were trypsinized and harvested as single cell suspension. Feeders were removed as mentioned above and approximately 300,000 epithelial cells were fixed and permeabilized by using Fixation/Permeabilization Solution Kit (BD biosciences, USA, cat. 554714). After a blocking procedure with Permeabilization solution at 4° C. for 30 min, cells were incubated with primary and Alexa Fluor 488 Secondary antibodies (Thermo Fisher, USA) for 1 hr at 4° C., with five washing events between each step. Primary antibodies used in these experiments include: mouse monoclonal anti-Ceacam5 antibody (MAB41281; Novus Biologicals, USA), rabbit polyclonal VSIG1 antibody (HPA036310; Sigma-Aldrich, USA) and mouse monoclonal anti-human PSCA antibody (sc-80654; Santa-Cruz Biotechnology, USA). Samples were collected and analyzed with on a Sony SH800S Cell Sorter (Sony Biotechnology, USA).

RNA Sample Preparation

For stem cell colonies, RNA was isolated using PicoPure RNA Isolation Kit (Life Technologies, USA). For ALI structure, RNA was isolated using Trizol RNA Isolation Kit (Life Technologies, USA). RNA quality (RNA integrity number, RIN) was measured by analysis Agilent 2100 Bioanalyzer and Agilent RNA 6000 Nano Kit (Agilent Technologies, USA). RNAs having a RIN>8 were used for microarray analysis.

Sequence Alignment of Single Cell RNA Sequencing

The single cell mRNA sequencing (scRNA-seq) libraries were established using the 10×Genomics Chromium system (Single Cell 3′ Reagent Kit v2). The scRNA-seq libraries were sequenced on the Illumina HiSeq X Ten with 10K cells for Crohn's case and fetal TI case. For normal case, the scRNA-seq library was sequenced on the Illumina NextSeq 500 with 2K cells. Demultiplexing, alignment and UMI-collapsing were performed using the Cellranger toolkit (version 2.1.0, 10×Genomics)⁵². The raw paired-end reads were trimmed to 26 bps for Read1 and 98 bps for Read2. The trimmed reads were mapped to both the human genome (hg19) and the mouse genome (mm10). The reads uniquely mapped to the human genome were used for downstream analysis.

Single Cell RNA Sequencing

The scRNA-seq data analyses were performed using the Seurat package (version 2.3.4)⁵³. We kept the genes with expression in at least three cells, and excluded cells expressing less than 200 genes. We identify the cell with SOX9 high expression as stem cell and also excluded the cells with high mitochondrial percentage or with an outlier level of UMI content. The normalization was performed using the global-scaling normalization method, which normalizes the gene expression measurements for each cell by the total expression, and then multiplies by 10,000, and finally log-transforms the result. The variable genes were identified using a function to calculate average expression and dispersion for each gene, divides these genes into bins, and then calculates a z-score for dispersion within each bin (“x.low.cutoff=0.0125”, “x.high.cutoff=3”, and “y.cutoff=0.5”). We scaled the data to regress out the variation of mitochondrial gene expression.

We performed PCA based on the scaled data to identify significant principal components (PCs). We selected the PCs with p-values less than 0.01 as input to perform clustering analysis and visualization by t-SNE. We detected the marker genes in each cell subpopulation using two methods of Wilcoxon rank sum test and DESeq2. For Wilcoxon rank sum test, we used the default parameter. For DEseq2, we kept the marker genes with average log-fold change above 0.1 and adjust p-value fewer than 0.05.

Contaminating 3T3-J2 fibroblast cells were identified by murine reads. In addition, the cells in S stage of cell cycle were identified based on the marker gene of SLBP⁵⁴. The cells in G2 or M stage of cell cycle were identified based on the marker genes of UBE2C, AURKA, CENPA, CDC20, HMGB2, CKS2, and CKS1B. The cells in GO stage of cell cycle were identified based on the marker genes of G0S2. In addition, the ambiguous cells with few marker genes were also removed, which could possibly correspond to sequencing low quality cells. Finally, we integrated the clean data of normal and Crohn's cases to perform clustering analysis and visualization by t-SNE.

Expression Microarray Data Analysis

Total RNAs obtained from immature colonies and ALI-differentiated epithelia were used for microarray preparation with WT Pico RNA Amplification System V2 for amplification of DNA and Encore Biotin Module for fragmentation and biotin labeling (NuGEN Technologies, USA). All samples were prepared according to manufacturer's instructions and hybridized onto GeneChip Human Exon 1.0 ST array (Affymetrix, USA). GeneChip operating software was used to process Cel files and calculate probe intensity values. To validate sample quality, quality checks were conducted using Affymetrix Expression Console software. The intensity values were log 2-transformed and imported into the Partek Genomics Suite 6.6 (Partek Incorporated, USA)⁵⁵. Exons were summarized to genes and a 1-way ANOVA was performed to identify differentially expressed genes. The heatmaps with hierarchical clustering analysis of the global gene expression pattern in different regions were performed using pheatmap package⁵⁶ (https://cran.rproject.org/web/packages/pheatmap/index.html) in R (version 3.5.1). The pathway enrichment analysis was performed using Enrichr⁵⁷ based on WikiPathways⁵⁸ database, and tissue enrichment analysis performed using ARCHS4 Tissues⁵⁹. The network analysis was constructed using ClueGO (v 2.5.4)⁶⁰ and CluePedia (v 1.5.4)⁶¹ plug-ins of Cytoscape⁶², based on the KEGG⁶³ and Reactome database⁶⁴.

Statistical Analysis

Unpaired two-tailed student's t-test was used to determine the statistical significance between two groups. Statistical analyses were performed using R (version 3.5.1). The “n” numbers for each experiment are provided in the text and figures. P<0.05 was considered statistically significant. Asterisks denote corresponding statistical significance *p<0.05; **p<0.01; ***p<0.001 and ****p<0.0001.

### REFERENCES FOR EXAMPLE 2

1. Khor, B., Gardet, A., and Xavier, R. J. Genetics and pathogenesis of inflammatory bowel disease. Nature 474, 307-317 (2011).
2. Baumgart, D. C., and Sandborn, W. J. Crohn's disease. Lancet 380, 1590-1605.
3. Neurath, M. (2017). Current and emerging therapeutic targets for IBD. Nat Rev Gastroenterol Hepatol 14, 688 (2012).
4. Halfvarson, J. Genetics in twins with Crohn's disease: less pronounced than previously believed? Inflamm Bowel Dis 17, 6-12 (2011).
5. Jager, S., Stange, E. F., and Wehkamp, J. Inflammatory bowel disease: an impaired barrier disease. Langenbecks Arch Surg 398, 1-12 (2013).
6. McGovern, D. P., Kugathasan, S., and Cho, J. H. Genetics of Inflammatory Bowel Diseases. Gastroenterology 149, 1163-1176 (2015).
7. Hampe, J., Franke, A., Rosenstiel, P., Till, A., Teuber, M., Huse, K., Albrecht, M., Mayr, G., De La Vega, F. M., Briggs, J., et al. A genome-wide association scan of nonsynonymous SNPs identifies a susceptibility variant for Crohn disease in ATG16L1. Nat Genet 39, 207-211 (2007).
8. Rioux, J. D., Xavier, R. J., Taylor, K. D., Silverberg, M. S., Goyette, P., Huett, A., Green, T., Kuballa, P., Barmada, M. M., Datta, L. W., et al. Genome-wide association study identifies new susceptibility loci for Crohn disease and implicates autophagy in disease pathogenesis. Nat Genet 39, 596-604 (2007).
9. Barrett, J. C., Hansoul, S., Nicolae, D. L., Cho, J. H., Duerr, R. H., Rioux, J. D., Brant, S. R., Silverberg, M. S., Taylor, K. D., Barmada, M. M., et al. Genome-wide association defines more than 30 distinct susceptibility loci for Crohn's disease. Nat Genet 40, 955-962 (2008).
10. Franke, A., McGovern, D. P., Barrett, J. C., Wang, K., Radford-Smith, G. L., Ahmad, T., Lees, C. W., Balschun, T., Lee, J., Roberts, R., et al. Genome-wide meta-analysis increases to 71 the number of confirmed Crohn's disease susceptibility loci. Nat Genet 42, 1118-1125 (2010).
11. Jostins, L., Ripke, S., Weersma, R. K., Duerr, R. H., McGovern, D. P., Hui, K. Y., Lee, J. C., Schumm, L. P., Sharma, Y., Anderson, C. A., et al. Host-microbe interactions have shaped the genetic architecture of inflammatory bowel disease. Nature 491, 119-124 (2012).
12. Liu, J. Z., van Sommeren, S., Huang, H., Ng, S. C., Alberts, R., Takahashi, A., Ripke, S., Lee, J. C., Jostins, L., Shah, T., et al. Association analyses identify 38 susceptibility loci for inflammatory bowel disease and highlight shared genetic risk across populations. Nat Genet 47, 979-986 (2015).
13. Wehkamp, J., Salzman, N. H., Porter, E., Nuding, S., Weichenthal, M., Petras, R. E., Shen, B., Schaeffeler, E., Schwab, M., Linzmeier, R., et al. Reduced Paneth cell alpha-defensins in ileal Crohn's disease. Proc Natl Acad Sci USA 102, 18129-18134 (2005).
14. Cadwell K, Liu J Y, Brown S L, Miyoshi H, Loh J, Lennerz J K, Kishi C, Kc W, Carrero J A, Hunt S, Stone C D, Brunt E M, Xavier R J, Sleckman B P, Li E, Mizushima N, Stappenbeck T S, Virgin H W 4th. A key role for autophagy and the autophagy gene Atg1611 in mouse and human intestinal Paneth cells. Nature 456, 259-263 (2008).
15. Saitoh, T., Fujita, N., Jang, M. H., Uematsu, S., Yang, B. G., Satoh, T., Omori, H., Noda, T., Yamamoto, N., Komatsu, M., et al. Loss of the autophagy protein Atg16L1 enhances endotoxin-induced IL-1beta production. Nature 456, 264-268 (2008).
16. Adolph, T. E., Tomczak, M. F., Niederreiter, L., Ko, H. J., Bock, J., Martinez-Naves, E., Glickman, J. N., Tschurtschenthaler, M., Hartwig, J., Hosomi, S., et al. Paneth cells as a site of origin for intestinal inflammation. Nature 503, 272-276 (2013).
17. Liu, T. C., Gurram, B., Baldridge, M. T., Head, R., Lam, V., Luo, C., Cao, Y., Simpson, P., Hayward, M., Holtz, M. L., et al. Paneth cell defects in Crohn's disease patients promote dysbiosis. JCI Insight 1, e86907 (2016).
18. Stappenbeck, T. S., and McGovern, D. P. B. Paneth Cell Alterations in the Development and Phenotype of Crohn's Disease. Gastroenterology 152, 322-326 (2017).
19. Abraham, C., and Cho, J. H. (2009). Inflammatory bowel disease. N Engl J Med 361, 2066-2078.
20. Farmer, R. G., Hawk, W. A., and Turnbull, R. B., Jr. Clinical patterns in Crohn's disease: a statistical study of 615 cases. Gastroenterology 68, 627-635 (1975).
21. Cleynen, I., Boucher, G., Jostins, L., Schumm, L. P., Zeissig, S., Ahmad, T., Andersen, V., Andrews, J. M., Annese, V., Brand, S., et al. Inherited determinants of Crohn's disease and ulcerative colitis phenotypes: a genetic association study. Lancet 387, 156-167 (2016).
22. Yamamoto, T. Factors affecting recurrence after surgery for Crohn's disease. World J Gastroenterol 11, 3971-3979 (2005).
23. Wang, X., Yamamoto, Y., Wilson, L. H., Zhang, T., Howitt, B. E., Farrow, M. A., Kern, F., Ning, G., Hong, Y., Khor, C. C., et al. Cloning and variation of ground state intestinal stem cells. Nature 522, 173-178 (2015).
24. Yamamoto, Y., Wang, X., Bertrand, D., Kern, F., Zhang, T., Duleba, M., Srivastava, S., Khor, C. C., Hu, Y., Wilson, L. H., et al. Mutational spectrum of Barrett's stem cells suggests paths to initiation of a precancerous lesion. Nat Commun 7, 10380 (2016).
25. Shultz, L. D., Brehm, M. A., Garcia-Martinez, J. V., and Greiner, D. L. Humanized mice for immune system investigation: progress, promise and challenges. Nat Rev Immunol 12, 786-798 (2012).
26. Kelly, S., Bliss, T. M., Shah, A. K., Sun, G. H., Ma, M., Foo, W. C., Masel, J., Yenari, M. A., Weissman, I. L., Uchida, N., et al. Transplanted human fetal neural stem cells survive, migrate, and differentiate in ischemic rat cerebral cortex. Proc Natl Acad Sci USA 101, 11839-11844 (2004).
27. Brazil, J. C., and Parkos, C. A. Pathobiology of neutrophil-epithelial interactions. Immunol Rev 273, 94-111 (2016).
28. West, N. R., Hegazy, A. N., Owens, B. M. J., Bullers, S. J., Linggi, B., Buonocore, S., Coccia, M., Gortz, D., This, S., Stockenhuber, K., et al. Oncostatin M drives intestinal inflammation and predicts response to tumor necrosis factor-neutralizing therapy in patients with inflammatory bowel disease. Nat Med 23, 579-589 (2017).
29. Schneider, W. M., Chevillotte, M. D., and Rice, C. M. Interferon-stimulated genes: a complex web of host defenses. Annu Rev Immunol 32, 513-545 (2014).
30. Fava, V. M., Dallmann-Sauer, M., and Schurr, E. Genetics of leprosy: today and beyond. Hum Genet 139, 835-846 (2020).
31. Vannberg, F. O., Chapman, S. J., and Hill, A. V. Human genetic susceptibility to intracellular pathogens. Immunol Rev 240, 105-116 (2011).
32. Raychaudhuri, S., Plenge, R. M., Rossin, E. J., Ng, A. C., International Schizophrenia, C., Purcell, S. M., Sklar, P., Scolnick, E. M., Xavier, R. J., Altshuler, D., et a. Identifying relationships among genomic disease regions: predicting genes at pathogenic SNP associations and rare deletions. PLoS Genet 5, e1000534 (2009).
33. Burke, J. P., Mulsow, J. J., O'Keane, C., Docherty, N. G., Watson, R. W., and O'Connell, P. R. Fibrogenesis in Crohn's disease. Am J Gastroenterol 102, 439-448 (2007).
34. D'Haens, G., Rieder, F., Feagan, B. G., Higgins, P. D. R., Panes, J., Maaser, C., Rogler, G., Lowenberg, M., van der Voort, R., Pinzani, M., et al. Challenges in the pathophysiology, diagnosis and management of intestinal fibrosis in inflammatory bowel disease. Gastroenterology pii: 50016-5085(19)₄₁₀₃₅-4. (2019).
35. Araya, J., and Nishimura, S. L. Fibrogenic reactions in lung disease. Annu Rev Pathol 5, 77-98 (2010).
36. Li, C., and Kuemmerle, J. F. The fate of myofibroblasts during the development of fibrosis in Crohn's Disease. J Dig Dis. 21, 326-331 (2020).
37. Ihara S, Hirata Y, Koike K. TGF-β in inflammatory bowel disease: a key regulator of immune cells, epithelium, and the intestinal microbiota. J Gastroenterol. 52, 777-787 (2017).
38. Liber, A. F. Aberrant pyloric glands in regional ileitis. AMA Arch Pathol 51, 205-212 (1951).
39. Wright N A, Pike C, Elia G. Induction of a novel epidermal growth factor-secreting cell lineage by mucosal ulceration in human gastrointestinal stem cells. Nature 343, 82-85 (1990).
40. Kariv, R., Plesec, T. P., Gaffney, K., Lian, L., Fazio, V. W., Remzi, F. H., Lopez, R., Goldblum, J. R., and Shen, B. Pyloric gland metaplasia and pouchitis in patients with ileal pouch-anal anastomoses. Aliment Pharmacol Ther 31, 862-873 (2010).
41. Thorsvik, S., van Beelen Granlund, A., Svendsen, T. D., Bakke, I., Royset, E. S., Flo, T. H., Damas, J. K., Ostvik, A. E., Bruland, T., and Sandvik, A. K. Ulcer-associated cell lineage expresses genes involved in regeneration and is hallmarked by high neutrophil gelatinase-associated lipocalin (NGAL) levels. J Pathol 248, 316-325 (2019).
42. Lloyd-Price, J., Arze, C., Ananthakrishnan, A. N., Schirmer, M., Avila-Pacheco, J., Poon, T. W., Andrews, E., Ajami, N. J., Bonham, K. S., Brislawn, C. J., et a. Multi-omics of the gut microbial ecosystem in inflammatory bowel diseases. Nature 569, 655-662 (2019).
43. Martin, J. C., Chang, C., Boschetti, G., Ungaro, R., Giri, M., Grout, J. A., Gettler, K., Chuang, L. S., Nayar, S., Greenstein, A. J., et al. Single-cell analysis of crohn's disease lesions identifies a pathogenic cellular module associated with resistance to anti-TNF therapy. Cell 178, 1493-1508 (2019).
44. Kaser, A., Zeissig, S., and Blumberg, R. S. Inflammatory bowel disease. Annu Rev Immunol 28, 573-621 (2010).
45. Filippi, M. D. Mechanism of diapedesis: Importance of the transcellular route. Adv Immunol 129, 25-53 (2016).
46. Rieder, F., Fiocchi, C., and Rogler, G. Mechanisms, management, and treatment of fibrosis in patients with inflammatory bowel diseases. Gastroenterology 152, 340-350 (2017).
47. Neurath, M. F. Targeting immune cell circuits and trafficking in inflammatory bowel disease. Nat Immunol 20, 970-979 (2019).
48. Tanoue, T., Atarashi, K., and Honda, K. Development and maintenance of intestinal regulatory T cells. Nat Rev Immunol 16, 295-309 (2016).
49. Zhang F R, Huang W, Chen S M, Sun L D, Liu H, Li Y, Cui Y, Yan X X, Yang H T, Yang R D et al., Genome-wide association study of leprosy. N Engl J Med. 361, 2609-2618 (2009).
50. Rao, W., Wang, S., Duleba, M., Niroula, S., Goller, K., Xie, J., Mahalingam, R., Neupane, R., Liew, A. A., Vincent, M., et al. Regenerative Metaplastic Clones in COPD Lung Drive Inflammation and Fibrosis. Cell 181, 848-864 (2020).
51. Duleba, M., Qi, Y., Mahalingam, R., Liew, A. A., Neupane, R., Flynn, K., Rinaldi, F., Vincent, M., Crum, C. P., Ho, K. Y., et al. An Efficient Method for Cloning Gastrointestinal Stem Cells From Patients via Endoscopic Biopsies. Gastroenterology 156, 20-23 (2019).
52. Ferguson A, Chen K. Analysis of Transcriptional Profiling of Immune Cells at the Single-Cell Level. Methods Mol Biol. 2111, 47-57 (2020).
53. Satija, R., Farrell, J. A., Gennert, D., Schier, A. F., and Regev, A. Spatial reconstruction of single-cell gene expression data. Nat Biotechnol 33, 495-502 (2015).
54. Nestorowa, S. et al. A single-cell resolution map of mouse hematopoietic stem and progenitor cell differentiation. Blood 128, e20-31, doi:10.1182/blood-2016-05-716480 (2016).
55. Wodehouse, T., Demopoulos, M., Petty, R., Miraki-Moud, F., Belhaj, A., Husband, M., Fulton, L., Randive, N., Oksche, A., Mehta, V., et al. A randomized pilot study to investigate the effect of opioids on immunomarkers using gene expression profiling during surgery. Pain 160, 2691-2698 (2019).
56. Li, G. M., Zhang, C. L., Rui, R. P., Sun, B., and Guo, W. Bioinformatics analysis of common differential genes of coronary artery disease and ischemic cardiomyopathy. Eur Rev Med Pharmacol Sci 22, 3553-3569 (2018).
57. Chen E Y, Tan C M, Kou Y, Duan Q, Wang Z, Meirelles G V, Clark N R, Ma'ayan A. Enrichr: interactive and collaborative HTML5 gene list enrichment analysis tool. BMC Bioinformatics. 14:128 (2013).
58. Slenter, D. N., Kutmon, M., Hanspers, K., Riutta, A., Windsor, J., Nunes, N., Melius, J., Cirillo, E., Coort, S. L., Digles, D., et al. WikiPathways: a multifaceted pathway database bridging metabolomics to other omics research. Nucleic Acids Res 46, D661-D667 (2018).
59. Lachmann, A., Torre, D., Keenan, A. B., Jagodnik, K. M., Lee, H. J., Wang, L., Silverstein, M. C., and Ma'ayan, A. (2018). Massive mining of publicly available RNA-seq data from human and mouse. Nat Commun 9, 1366.
60. Bindea, G., Mlecnik, B., Hackl, H., Charoentong, P., Tosolini, M., Kirilovsky, A., Fridman, W.-H., Pages, F., Trajanoski, Z., and Galon, J. ClueGO: a Cytoscape plug-in to decipher functionally grouped gene ontology and pathway annotation networks. Bioinformatics 25, 1091-1093 (2009).
61. Bindea, G., Galon, J., and Mlecnik, B. CluePedia Cytoscape plugin: pathway insights using integrated experimental and in silico data. Bioinformatics 29, 661-663 (2013).
62. Shannon, P., Markiel, A., Ozier, O., Baliga, N. S., Wang, J. T., Ramage, D., Amin, N., Schwikowski, B., and Ideker, T. Cytoscape: a software environment for integrated models of biomolecular interaction networks. Genome Res 13, 2498-2504 (2003).
63. Kanehisa, M., and Goto, S. KEGG: kyoto encyclopedia of genes and genomes. Nucleic Acids Res 28, 27-30 (2000).
64. Wu, G., and Haw, R. Functional Interaction Network Construction and Analysis for Disease Discovery. Methods Mol Biol 1558, 235-253 (2017).

### Example 3-Methods

In vitro culture of human terminal ileum and colonic epithelial stem cells. Terminal ileum endoscopic biopsies were obtained from pediatric Crohn's patients and functional controls lacking mucosal inflammation under informed parental consent and institutional review board approval at the Connecticut Children's Medical Center, Hartford, Conn. USA and the University of North Carolina, Chapel Hill, Chapel Hill, N.C., USA. 1 mm endoscopic biopsies were collected into cold F12 media (Gibco, USA) with 5% fetal bovine serum (Hyclone, USA), and then were minced by sterile scalpel into 0.2-0.5 mm³ fragments. The minced tissue was digested in 2 mg/ml collagenase type IV (Gibco, USA) at 37° C. for 30-60 min with agitation. Dissociated cells were passed through a 70 μm Nylon mesh (Falcon, USA) to remove masses and then were washed four times in cold F12 media, and seeded onto a feeder layer of lethally irradiated 3T3-J2 cells in c-FAD media (51) containing 125 ng/mL R-Spondin1 (R&D systems, USA), 1 μM Jagged-1 (AnaSpec Inc, USA), 100 ng/ml Human Noggin (Peprotech, USA), 2.5 μM Rock-inhibitor (Calbiochem, USA), 2 μM SB431542 (Cayman chemical, USA), 10 mM nicotinamide (Sigma-Aldrich, USA). Cells were cultured at 37° C. in a 7.5% CO₂ incubator. The culture media was changed every two days. Colonies were digested by 0.25% trypsin-EDTA solution (Gibco, USA) for 5-8 min and passaged every 7 to 10 days. Colonies were trypsinized by TrypLE Express solution (Gibco, USA) for 8-15 min at 37° C. and cell suspensions were passed through 30 μm filters (Miltenyi Biotec, Germany). Approximately 20,000 epithelial cells were seeded to each well of 6-well plate. Cloning cylinder (Pyrex, USA) and high vacuum grease (Dow Corning, USA) were used to select single colonies for pedigrees. Gene expression analyses were performed on cells derived from passage 4-10 (P4-P10) cultures.

Histology and Immunostaining. Histology, hematoxylin and eosin (H&E) staining, immunohistochemistry, and immunofluorescence were performed using standard techniques. For immunofluorescence and immunohistochemistry, 4% paraformaldehyde-fixed, paraffin embedded tissue slides were subjected to antigen retrieval in citrate buffer (pH 6.0, Sigma-Aldrich, USA) at 120° C. for 20 min, and a blocking procedure was performed with 5% bovine serum albumin (BSA, Sigma-Aldrich, USA) and 0.05% Triton X-100 (Sigma-Aldrich, USA) in DPBS(−) (Gibco, USA) at room temperature for 1 hr. The sources of primary antibodies used in this study, including anti-mucin 2, -Ki67, -chromogranin A, -alpha defensin 6, -E-cadherin, and -GPA33 are listed (table S3). All images were captured by using the Inverted Eclipse Ti-Series (Nikon, Japan) microscope with Lumencor SOLA light engine and Andor Technology Clara Interline CCD camera and NIS-Elements Advanced Research v.4.13 software (Nikon, Japan) or LSM 780 confocal microscope (Carl Zeiss, Germany) with LSM software. Bright field cell culture images were obtained on an Eclipse TS100 microscope (Nikon, Japan) with Digital Sight DSFilcamera (Nikon, Japan) and NIS-Elements F3.0 software (Nikon, Japan).

Stem cell differentiation. Air-liquid interface (ALI) culture of terminal ileum epithelial cells was performed as described (21,22). Briefly, Transwell inserts (Corning Incorporated, USA) were coated with 20% Matrigel (BD biosciences, USA) and incubated at 37° C. for 30 min to polymerize. 200,000 irradiated 3T3-J2 cells were seeded to each Transwell insert and incubated at 37° C., 7.5% CO2 incubator overnight. QuadroMACS Starting Kit (LS) (Miltenyi Biotec, Germany) was used to purify the stem cells by removal of feeder cells. 200,000-300,000 stem cells were seeded into each Transwell insert and cultured with stem cell media. At confluency (3-7 days), the apical media on the inserts was removed through careful pipetting and the cultures were continued in differentiation media (stem cell media without nicotinamide) for an additional 6-12 days prior to harvesting. The differentiation media was changed every one or two days.

RNA sample preparation. For stem cell colonies, RNA was isolated using PicoPure RNA Isolation Kit (Life Technologies, USA). For ALI structure, RNA was isolated using Trizol RNA Isolation Kit (Life Technologies, USA). RNA quality (RNA integrity number, RIN) was measured by analysis Agilent 2100 Bioanalyzer and Agilent RNA 6000 Nano Kit (Agilent Technologies, USA). RNAs having a RIN>8 were used for microarray analysis.

Expression microarray and bioinformatics. Total RNAs obtained from immature colonies and ALI differentiated epithelia were used for microarray preparation with WT Pico RNA Amplification System V2 for amplification of DNA and Encore Biotin Module for fragmentation and biotin labeling (NuGEN Technologies, USA). All samples were prepared according to manufacturer's instructions and hybridized onto GeneChip Human Exon 1.0 ST or Human Transcriptome (HTA) Arrays (Affymetrix, USA). GeneChip operating software was used to process all the Cel files and calculate probe intensity values. To validate sample quality, quality checks were conducted using Affymetrix Expression Console software. The intensity values were log 2-transformed and imported into the Partek Genomics Suite 6.6 (Partek Incorporated, USA). Exons were summarized to genes and a 1-way ANOVA was performed to identify differentially expressed genes. Unsupervised clustering and heatmap generation were performed with sorted datasets by Euclidean distance based on average linkage clustering, and Principal Component Analysis (PCA) map was conducted using all or selected probe sets by Partek Genomics Suite 6.6. Pathway analyses were performed with Ingenuity Pathway Analysis (IPA; 52) software.

The fetal gastrointestinal whole genome microarray data were grouped as stomach (gastric fundus, body, antrum), small intestine (duodenum, jejunum, ileum) and colon (ascending, transverse, and descending colon). These groups of data were normalized together and three differential gene expression analyses (stomach vs small intestine, small intestine vs colon and stomach vs colon) were performed and then comparisons combined to make the final differentially expressed genes list and heatmap. To make the heatmap of the Crohn's over- and under-represented genes along the fetal gastrointestinal tract, only those genes were selected from the final fetal gastrointestinal differentially expressed genes and then plotted as the heatmap. Datasets used in this study have been deposited with the National Center for Biotechnology Information Gene Expression Omnibus (GEO) database under files GSE57584 and GSE89045.

Genome editing. To target the ATOH1 coding sequence, an sgRNA targeting sequence CCTGCTGCATGCAGAAGAGT (SEQ ID NO: 1) was generated in the plasmid pSpCas9(BB)-2A-GFP (PX458; GeneScript) driving Cas9 and GFP expression. Plasmid DNA was amplified and transiently transfected into mucosal stem cells with the Neon® Transfection System (Invitrogen) following the manufacturer's protocol (1,300V, 30 ms, 1 pulse). Cells were counted and re-suspended in 100 ul Buffer R to a final concentration of 4×10⁶ cells ml⁻¹, and then mixed with 30 ug DNA. Eighty-four hours after transfection, GFP-positive cells were sorted (BD FACS Diva, BD Biosciences) and recovered in 12-well plates. Individual colonies were picked and their genomic DNA was extracted (DNeasy Blood & Tissue Kits, QIAGEN). The genomic region surrounding the CRISPR target site of ATOH1 was PCR amplified, and its products were purified (QiaQuick Gel Extraction Kit, Qiagen) for mutation analysis by sequencing (Eurofins Genomics, USA).

Retroviral infection for ATOH1 overexpression. PCR-amplified hATOH1 fragments were cloned into pMX-IRES-eGFP retroviral vector between AscI and PacI sites (Addgene). The pseudotyping vector pVSV-G and pMX-hATOH1 plasmid was transfected into the GP2-293 retroviral packaging cell line following the manufacturer's protocol (jetPRIME, Polyplus transfection). Virus was then harvested and concentrated 48 hours later to titers are around 1×10⁷ pfu/ml. Retroviral infection was performed when the mucosal stem cell colonies were still small (less than 100 cells), and the MOI (multiplicity of infection) value is around 10. Infected stem cells, including both GFP-positive and GFP-negative ones, were isolated from feeder cells as described and seeded into Transwell inserts (Corning, USA).

Epigenomics. Chromatin immunoprecipitations (53-55) were performed using antibodies against H3K4me3 (07-473; Millipore, USA), H3K27ac (ab4729; Abcam, UK), H3K27me3 (07-449; Millipore, USA) against chromatin prepared from 1-5 million mucosal stem cells. DNA libraries were generated using the TruSeq ChIP-sequencing kit (Illumina, USA) and sequenced in a multiplexed format on a HiSeq2000 with 36 bp single-end reads (Illumina, USA). Raw reads were trimmed using Trim Galore (world-wide-web at bioinformatics.babraham.ac.uk/projects/trim_galore/). Potential mouse genomic DNA contaminant reads were removed from further analysis using Xenome (56). Trimmed reads were uniquely mapped to the reference genome (UCSC hg19) using Bowtie 2 (version 2.1.0) (57). Peaks for each sample were identified using MACS2 (version 2.1.1) (58) by comparing its corresponding input control with parameters callpeak --keep-dup 1 --nomodel --extsize 147 for H3K4me3 and H3K27ac (--extsize 325 for H3K27me3). The resulted peaks were filtered according to the blacklist of ENCODE (59). IGV (60) was used to visiualize peak profiles based on the bigwig files converted from the result of MACS2. Differential binding event analysis was assessed using bdgdiff of MACS2. The unique peak in only one condition was detected using intersectBed of bedtools (61) based on the peaks identified by MACS2. The final set of differential binding peaks that were combined the results of two software was filtered by the region in 5 kb upstream/downstream of TSS (transcriptional start site). These peaks were annotated using R Bioconductor package ChlPpeakAnno (62). The region for each peak around peak summit (+/−500-bp) and mean values were calculated for 50 bp bins uisng computeMatrix module of deepTools2 (63). Based this matrix, the heatmap with hierarchical clustering and the average profile were calculated using plotHeatmap and plotProfile modules of deepTools2 in seperate.

### REFERENCES IN METHODS

The following references, to the extent that they provide exemplary procedural or other details supplementary to those set forth herein, are specifically incorporated herein by reference.
51. Rheinwald J G, Green H. Serial cultivation of strains of human epidermal keratinocytes: the formation of keratinizing colonies from single cells. Cell 6, 331-343 (1975).
52. Mayburd A L, Martlinez A, Sackett D, Liu H, Shih J, Tauler J, Avis I, Mulshine J L. Ingenuity network-assisted transcription profiling: Identification of a new pharmacologic mechanism for MK886. Clin Cancer Res. 2006 12, 1820-1827 (2006).
53. Zhang B, Zheng H, Huang B, Li W, Xiang Y, Peng X, Ming J, Wu X, Zhang Y, Xu Q Liu W, Kou X, Zhao Y, He W, Li C, Chen B, Li Y, Wang Q, Ma J, Yin Q, Kee K, Meng A, Gao S, Xu F, Na J, Xie W. Allelic reprogramming of the histone modification H3K4me3 in early mammalian development. Nature 537, 553-557 (2016).
54. Lin C Y, Erkek S, Tong Y, Yin L, Federation A J, Zapatka M, Haldipur P, Kawauchi D, Risch T, Warnatz H J, Worst B C, Ju B, Orr B A, Zeid R, Polaski D R, Segura-Wang M, Waszak S M, Jones D T, Kool M, Hovestadt V, Buchhalter I, Sieber L, Johann P, Chavez L, Gröschel S, Ryzhova M, Korshunov A, Chen W, Chizhikov V V, Millen K J, Amstislavskiy V, Lehrach H, Yaspo M L, Eils R, Lichter P, Korbel J O, Pfister S M, Bradner J E, Northcott P A. Active medulloblastoma enhancers reveal subgroup-specific cellular origins. Nature 530, 57-62 (2016).
55. Liu X, Wang C, Liu W, Li J, Li C, Kou X, Chen J, Zhao Y, Gao H, Wang H, Zhang Y, Gao Y, Gao S. Distinct features of H3K4me3 and H3K27me3 chromatin domains in pre-implantation embryos. Nature 537, 558-562 (2016).
56. Thomas Conway, Jeremy Wazny, Andrew Bromage, Martin Tymms, Dhanya Sooraj, Elizabeth D. Williams and Bryan Beresford-Smith. Xenome—a tool for classifying reads from xenograft samples. Bioinformatics 28, i172-i178 (2012).
57. Langmead B and Salzberg S L. Fast gapped-read alignment with Bowtie 2. Nat Methods 9, 357-359 (2012).
58. Zhang Y, Liu T, Meyer C A, Eeckhoute J, Johnson D S, Bernstein B E, Nusbaum C, Myers R M, Brown M, Li W, Liu X S. Model-based analysis of ChIP-Seq (MACS). Genome Biol. 9, R137 (2008).
59. ENCODE Project Consortium. An integrated encyclopedia of DNA elements in the human genome. Nature 489, 57-74 (2012).
60. James T. Robinson, Helga Thorvaldsdóttir, Wendy Winckler, Mitchell Guttman, Eric S. Lander, Gad Getz, Jill P. Mesirov. Integrative Genomics Viewer. Nature Biotechnology 29, 24-26 (2011).
61. Aaron R. Quinlan and Ira M. Hall. BEDTools: a flexible suite of utilities for comparing genomic features. Bioinformatics 2010; 26(6): 841-842 (2010).
62. Zhu L, Gazin C, Lawson N, Pages H, Lin S, Lapointe D and Green M. ChlPpeakAnno: a Bioconductor package to annotate ChIP-seq and ChIP-chip data. BMC Bioinformatics (2010) 11, 237 (2010).
63. Ramirez F, Ryan D P, Gruning B, Bhardwaj V, Kilpert F, Richter A S, Heyne S, Dundar F, Manke T. deepTools2: a next generation web server for deep-sequencing data analysis. Nucleic Acids Research. 2016 Apr. 13:gkw257.

All of the methods disclosed and claimed herein can be made and executed without undue experimentation in light of the present disclosure. While the compositions and methods of this disclosure have been described in terms of particular embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

1. A method for treating a patient presenting with one or more of an inflammatory disease, metaplasia, dysplasia or cancer of gastrointestinal tissues which method comprises administering to the patient an agent that selectively kills or inhibits the proliferation or differentiation of pathogenic epithelial stem cells (PESCs) relative to normal gastrointestinal stem cells.

2. A method of reducing proliferation, survival, migration, or colony formation ability of pathogenic epithelial stem cells (PESCs) in a subject in need thereof comprising contacting the cell with a therapeutically effective amount of an agent that selectively kills or inhibits the proliferation or differentiation of PESC relative to normal gastrointestinal stem cells.

3. A pharmaceutical preparation for treating one or more of an inflammatory disease, metaplasia, dysplasia or cancer of gastrointestinal tissues, the preparation comprises an agent that selectively kills or inhibits the proliferation or differentiation of pathogenic epithelial stem cells (PESCs) relative to normal gastrointestinal stem cells.

4. A drug eluting device for treating one or more of an inflammatory disease, metaplasia, dysplasia or cancer of gastrointestinal tissues, which device comprises drug release means including an agent that selectively kills or inhibits the proliferation or differentiation of pathogenic epithelial stem cells (PESCs) relative to normal gastrointestinal stem cells, which device when deployed in a patient positions the drug release means proximal to the luminal surface of the gastrointestinal tissue and releases the agent in an amount sufficient to achieve a therapeutically effective exposure of the luminal surface to the agent.

5. The method of claim 1, for the treatment of inflammatory bowel disease.

6. The method of claim 1, for the treatment of Crohn's Disease or Perinal Crohn's Disease.

7. The method of claim 1, wherein the agent is administered as part of a therapy including administration of one or more anti-inflammatory agents.

8. The method of claim 1, wherein the agent is administered by submucosal injection of gastrointestinal tissue.

9. The preparation of claim 3, wherein the agent is formulated for submucosal injection of gastrointestinal tissue.

10. The method of claim 8, wherein the agent is formulated as part of a bioadhesive formulation.

11. The method of claim 8, wherein the agent is formulated as part of a drug-eluting particle, drug eluting matrix or drug-eluting gel.

12. The method of claim 1, wherein the agent is administered by topical application to gastrointestinal tissue.

13. The preparation of claim 3, wherein the agent is formulated for topical application to gastrointestinal tissue.

14. The method of claim 12, wherein the agent is formulated as part of a bioadhesive formulation.

15. The method of claim 12, wherein the agent is formulated as part of a drug-eluting particle, drug eluting matrix or drug-eluting gel.

16. The method of claim 12, wherein the agent is formulated for oral delivery and release in gastrointestinal tract.

17. The method or preparation of claim 16, wherein the agent is formulated for oral delivery and release in the terminal ileum.

18. The method of claim 1, wherein the agent selectively inhibits the proliferation or differentiation of PESCs, or selectively kills PESCs, with an IC₅₀ that is ⅕^{th} or less the IC50 for normal gastrointestinal stem cells, more preferably 1/10^{th}, 1/20^{th}, 1/50^{th}, 1/100^{th}, 1/250^{th}, 1/500^{th} or even 1/1000^{th}.

19. The method of claim 1, wherein the agent inhibits the proliferation or differentiation of PESCs, or kills PESCs, with an IC₅₀ of 10⁻⁶ M or less, more preferably 10⁻⁷ M or less, 10⁻⁸ M or less or 10⁻⁹ M or less.

20. The method of claim 1, wherein the agent is an HSP90 inhibitor, an HSP70 inhibitor or a dual HSP90/HSP70 inhibitor.

21. The method of claim 1, wherein the agent is an mTOR inhibitor.

22. The method of claim 1, wherein the agent is an RAR antagonist.

23. The method of claim 1, wherein the agent is a proteasome inhibitor.

24. The method, preparation or device of any of claim 23, wherein the a proteasome inhibitor, is an immunoproteasome inhibitor.

25. The method of claim 1, wherein the agent is a BCR-ABL kinase inhibitor.

26. The method of claim 1, further comprises combining the agent with a second drug agent that selectively promotes proliferation of normal gastrointestinal stem cells with an EC₅₀ at least 5 times more potent than for PESCs, more preferably with an EC₅₀10 times, 50 times, 100 times or even 1000 times more potent than for PESCs.

27. The method, preparation or device of claim 26, wherein the second drug agent promotes proliferation of normal gastrointestinal stem cells with an EC₅₀ of 10⁻⁶ M or less, more preferably 10⁻⁷ M or less, 10⁻⁸ M or less or 10⁻⁹ M or less.

28. The method, preparation or device of claim 26, wherein the second drug agent is a BACE inhibitor

29. The method, preparation or device of claim 28, wherein the BACE inhibitor is a BACE1 inhibitor.

30. The method, preparation or device of claim 26, wherein the second drug agent is an FAK Inhibitor.

31. The method, preparation or device of claim 26, wherein the second drug agent is a VEGFR inhibitor.

32. The method, preparation or device of claim 26, wherein the second drug agent is an AKT inhibitor.

33. The method of claim 26, wherein the agent and the second agent are administered to the patient as separate formulations.

34. The method of claim 26, wherein the agent and the second agent are co-formulated together.

35. The method of claim 1 wherein the patient is a human patient.

36. A terminal ileum retentive formulation comprising (i) an agent that selectively kills or inhibits the proliferation or differentiation of pathogenic epithelial stem cells (PESCs) relative to normal gastrointestinal stem cells, (ii) a bioadhesive, and (iii) optionally, one or more pharmaceutically acceptable excipients.

37. The terminal ileum retentive formulation of claim 36, which formulation has a mucosal residence half-life on terminal ileum tissue of at least 120 minutes.

38. The terminal ileum retentive formulation of claim 6, which formulation produces at least a minimally effective concentration (MEC) of the agent in terminal ileum tissue to which it is applied for at least 120 minutes.

39. The terminal ileum retentive formulation of claim 36, which formulation produces agent concentration in terminal ileum tissue to which it is applied with T1i2 of at least 4 hours.

40. A perianal or anorectal retentive formulation comprising (i) an agent that selectively kills or inhibits the proliferation or differentiation of pathogenic epithelial stem cells (PESCs) relative to normal gastrointestinal stem cells, (ii) a bioadhesive, and (iii) optionally, one or more pharmaceutically acceptable excipients.

41. The perianal or anorectal retentive formulation of claim 40, which formulation has a mucosal residence half-life on terminal ileum tissue of at least 120 minutes.

42. The perianal or anorectal retentive formulation of claim 40, which formulation produces at least a minimally effective concentration (MEC) of the agent in perianal or anorectal tissue to which it is applied for at least 120 minutes.

43. The perianal or anorectal retentive formulation of claim 40, which formulation produces agent concentration in perianal or anorectal tissue to which it is applied with T1i2 of at least 4 hours.

44. The perianal or anorectal retentive formulation of claim 40, which formulation is a viscous bioadhesive liquid to coat the perianal or anorectal tissue.

45. The perianal or anorectal retentive formulation of claim 40, which formulation comprises agent eluting multiparticulates, microparticles, nanoparticles or microdiscs.

46. The perianal or anorectal retentive formulation of claim 40, which formulation includes one or more an HSP90 inhibitor, an HSP70 inhibitor, a dual HSP90/HSP70 inhibitor, an mTOR inhibitor, an RAR antagonist, a proteasome inhibitor, an EGFR inhibitor, a ROCK inhibitor, a MELK inhibitor, a SRC inhibitor and/or a BCR-ABL kinase inhibitor.

47. The perianal or anorectal retentive formulation of claim 46, which formulation further includes one or more a BACE inhibitor, an FAK inhibitor, a VEGR inhibitor and/or an AKT inhibitor.

48. The perianal or anorectal retentive formulation of claim 46, which formulation further includes one or more antihistamines, antipyretics, analgesics, anti-infective agents and/or chemotherapeutic agents.

46. A bioadhesive nanoparticle having a polymeric surface with an adhesive force equivalent to an adhesive force of between 10 N/m² and 100,000 N/m² measured on human mucosal surfaces, which nanoparticle further includes: (i) a first agent selected from an HSP90 inhibitor, an HSP70 inhibitor, a dual HSP90/HSP70 inhibitor, an mTOR inhibitor, an RAR antagonist, a proteasome inhibitor, an EGFR inhibitor, a ROCK inhibitor, a MELK inhibitor, a SRC inhibitor or a BCR-ABL kinase inhibitor; and (ii) a second agent selected from a BACE inhibitor, an FAK inhibitor, a VEGR inhibitor or an AKT inhibitor, the first and second agents dispersed therein or thereon, wherein the nanoparticle elutes the first and second agents into the mucous gel layer when adhered to mucosal tissue.

47. A submucosal retentive formulation comprising: (i) a first agent selected from an HSP90 inhibitor, an HSP70 inhibitor, a dual HSP90/HSP70 inhibitor, an mTOR inhibitor, an RAR antagonist, a proteasome inhibitor, an EGFR inhibitor, a ROCK inhibitor, a MELK inhibitor, a SRC inhibitor or a BCR-ABL kinase inhibitor; (ii) a second agent selected from a BACE inhibitor, an FAK inhibitor, a VEGR inhibitor or an AKT inhibitor; and (iii) one or more pharmaceutically acceptable excipients, which formulation is injectable submucosally and forms a submucusal depot releasing an effective amount of the first and second agents to the surrounding tissue.

48. An injectable thermogel for submucosal injection, comprising: (i) a first agent selected from an HSP90 inhibitor, an HSP70 inhibitor, a dual HSP90/HSP70 inhibitor, an mTOR inhibitor, an RAR antagonist, a proteasome inhibitor, an EGFR inhibitor, a ROCK inhibitor, a MELK inhibitor, a SRC inhibitor or a BCR-ABL kinase inhibitor; (ii) a second agent selected from a BACE inhibitor, an FAK inhibitor, a VEGR inhibitor or an AKT inhibitor; and optionally (iii) one or more pharmaceutically acceptable excipients, wherein the thermogel has a low-viscosity fluid at room temperature (and easily injected), and becomes a non-flowing gel at body temperature after injection.

49. The injectable thermogel of claim 48, which includes a poly(lactic acid-co-glycolic acid)-poly(ethylene glycol)-poly(lactic acid-co-glycolic acid) (PLGA-PEG-PLGA) triblock copolymers.

50. A drug eluting device for treating an inflammatory bowel disease, which device comprises drug release means including an agent that selectively kills or inhibits the proliferation or differentiation of pathogenic epithelial stem cells (PESCs) relative to normal gastrointestinal stem cells, which device when deployed in a patient positions the drug release means proximal to the luminal surface of the gastrointestinal tissue and releases the agent in an amount sufficient to achieve a therapeutically effective exposure of the luminal surface to the agent.

51. The drug eluting device of claim 50, wherein the agent is selected from an HSP90 inhibitor, an HSP70 inhibitor, a dual HSP90/HSP70 inhibitor, an mTOR inhibitor, an RAR antagonist, a proteasome inhibitor, an EGFR inhibitor, a ROCK inhibitor, a MELK inhibitor, a SRC inhibitor or a BCR-ABL kinase inhibitor, or a combination thereof.

52. The drug eluting device of claim 51, further comprising a second agent selected from a BACE inhibitor, an FAK inhibitor, a VEGR inhibitor or an AKT inhibitor.

53. The drug eluting device of claim 50, wherein drug eluting device is a biodegradable stent.

54. The drug eluting device of claim 53, wherein drug eluting device is a self-expandable stent, such as a self-expandable metallic stent (SEMS) or self-expandable plastic stent (SEPS).

55. The drug eluting device of claim 50, wherein drug eluting device is a chip or wafers for submucusal implantation.

56. The drug eluting device of claim 50, wherein drug eluting device is a device for extraluminal placement, such as a microneedle cuff.

57. Single oral dosage formulation comprising: (i) a first agent selected from an HSP90 inhibitor, an HSP70 inhibitor, a dual HSP90/HSP70 inhibitor, an mTOR inhibitor, an RAR antagonist, a proteasome inhibitor, an EGFR inhibitor, a ROCK inhibitor, a MELK inhibitor, a SRC inhibitor or a BCR-ABL kinase inhibitor; (ii) a second agent selected from a BACE inhibitor, an FAK inhibitor, a VEGR inhibitor or an AKT inhibitor; and (iii) and a pharmaceutically acceptable excipient, which single oral dosage formulation taken by an adult patient produces a concentration of the first and second agent in terminal ileum tissue effective to slow or reverse the progress of Crohn's disease.

58. A suppository formulation comprising a first agent selected from an HSP90 inhibitor, an HSP70 inhibitor, a dual HSP90/HSP70 inhibitor, an mTOR inhibitor, an RAR antagonist, a proteasome inhibitor, an EGFR inhibitor, a ROCK inhibitor, a MELK inhibitor, a SRC inhibitor or a BCR-ABL kinase inhibitor; and, optionally a second agent selected from a BACE inhibitor, an FAK inhibitor, a VEGR inhibitor or an AKT inhibitor; and (iii) and a pharmaceutically acceptable excipient, which single oral dosage formulation taken by an adult patient produces a concentration of the first and second agent in terminal ileum tissue effective to slow or reverse the progress of Crohn's disease.


SEQUENCE LISTING (1 sequence)

SEQ ID NO: 1 - DNA, 20 bp, Artificial sequence
  Synthetic nucleic acid
cctgctgcatgcagaagagt